(19) United States
(12) Patent Application Publication
(10) Pub. No.: US 2005/0271615 A1
(43) Pub. Date: Dec. 8, 2005

(54) Self-immolative dendrimers releasing many active moieties upon a single activating event

(76) Inventors: Doron Shabat, TEL AVIV (IL); Benjamin List, Muelheim an der Ruhr (DE); Roey Jacob Amir, Tel Aviv (IL); Marina Shamis, Hadera (IL); Neta Pessah, Tel Aviv (IL)

Correspondence Address: Martin Moynihan;c/o Anthony Castorina, 2001 Jefferson Davis Highway, Suite 207, Arlington, VA, 22202, US

(21) Appl. No.: 10/525,951

(22) PCT Filed: Aug. 28, 2003
(86) PCT No.: PCT/IL03/00711

§ 371 Date: Feb. 28, 2005

Related U.S. Application Data
(60) Provisional application No. 60/406,958, filed on Aug. 30, 2002.

(51) Int. Cl. A61K 31/785; C08L 77/06

(52) U.S. Cl. 424/78.3; 525/436

(57) ABSTRACT

A self-immolative dendrimer capable of releasing all of its tail units upon a single cleavage event, methods of synthesizing same and uses thereof are disclosed.

### FIELD AND BACKGROUND OF THE INVENTION

The present invention relates to novel dendrimeric compounds and, more particularly, to self-immolative dendrimers which can release a plurality of tail units upon a single cleavage event and can therefore be beneficially used in, for example, a variety of therapeutic and diagnostic applications.

Dendrimers are perfectly cascade-branched, highly defined, synthetic macromolecules, characterized by a combination of high-group functionalities and a compact molecular structure [1]. Dendrimers in general comprise a core, a number of generations of ramifications (also known and referred to herein as “branches” or “branching units”) and an external surface. The generations of ramifications are composed of repeating structural units, which radially extend outwardly from the dendrimer core. The external surface of a dendrimer of an Nth generation is, in general, composed of the terminal functional groups (also known and referred to herein as “end groups”, “tail groups” or “tail units”) of the Nth (final) generation. The concept of repetitive growth with branching creates a unique spherical mono-disperse dendrimer formation, which is defined by a precise generation number (Gn) [2]. For example: a first generation dendrimer (G1) will have one branching unit, a second generation (G2) will have additional two branching units, etc.

The size, shape and, inherently, the properties of a dendrimer molecule and the functional groups present in the dendrimer molecule can be controlled by the choice of the core, the number of generations, and the choice of the repeating units employed at each generation. Being a synthetic supermolecule, dendrimers can be designed to exert predetermined properties by selecting the appropriate components. For example, the core type can affect the dendrimer shape, producing, e.g., spheroid-shaped dendrimers, cylindrical- or rod-shaped dendrimers, or ellipsoid-shaped dendrimers. Sequential building of generations determines the dimensions of the dendrimers and the nature of its interior. The chemical functionality and structure of the repeating unit in the interior layers can affect, for example, the shape and dimension of the empty volumes between the ramifications.

The synthesis of dendrimers usually occurs by a divergent approach that involves the initial reaction of a monomer with the initiator core, followed by exhaustive reaction of the resulting functional groups with a difunctional compound, to afford the next generation of reactive groups. Repetition of the two-step procedure leads to subsequent generations. An alternate synthetic route uses a convergent growth synthesis as is described in detail in C. J. Hawker and J. M. J. Frechet, J. Am. Chem. Soc., 112,7638 (1990), the disclosure of which is incorporated by reference as if fully set forth herein.

The unique, precise and predetermined structure of dendrimers has been exploited in various fields such as, for example, energy transfer, light harvesting, dyes, nanoparticles, biological analogies, and as carriers of agricultural, pharmaceutical and other materials. Representative examples of dendrimeric compositions and their uses in a variety of fields are disclosed in U.S. Pat. Nos. 6,579,906, 6,570,031, 6,545,101, 6,506,218, 6,464,971, 6,452,053, 6,410,680, 6,395,257, 6,365,562, 6,312,809, 6,306,991, 6,288,253, 6,228,978, 6,224,898, 6,187,897, 6,184,313, 6,113,946, 6,083,708, 6,068,835, 5,990,089, 5,938,934, 5,902,863, 5,788,989, 5,736,346, 5,714,166, 5,661,025, 5,648,186, 5,393,797, 5,393,795, 5,332,640, 5,266,106, 5,256,516, 5,256,193, 5,098,475, 4,938,885 and 4,694,064.

The structural precision of dendrimers has therefore further motivated numerous studies regarding biological applications [3, 4]. Representative examples of such applications include the amplification of molecular effects and the creation of high concentrations of drugs [5], molecular labels, and probe moieties [6].

However, most of the presently known dendrimers' biological applications rely mainly on the high-group functionality and not on their unique structural perfection.

For example, dendrimers are used in chemotherapy treatment as prodrugs that selectively liberate a drug at the tumor site [7, 8]. This selectivity is achieved by using high molecular weight (of more than 20,000 Daltons) drug-dendrimer conjugates [9], and is based on the known ability of macromolecules to accumulate selectively at tumor sites due to the enhanced permeability and retention (EPR) effect [10].

The release of the drug from the presently known dendrimeric prodrugs is achieved by an approach that involves linking the drug to the dendrimers through an enzymatic cleavable linker [11]. Such an approach, which exploits the existence of tumor-specific enzymes, is widely used in designing anti-cancer prodrugs, and is based on the conversion of a pharmacologically inactive prodrug to the corresponding active drug in the vicinity of the tumor by a relatively high level of a specific enzyme that is targeted or secreted near the tumor cells.

An example of such a site-specific prodrug is disclosed, for example, in WO 02/083180, which is incorporated by reference as if fully set forth herein. WO 02/083180 discloses self-eliminating spacers that are incorporated between an enzymatically removable specifier and a parent drug. According to the teachings of WO 02/083180, the resulting prodrug exerts improved drug targeting to disease-related or organ-specific tissue or cells and facilitated release of the parent drug.

Nevertheless, although such prodrug systems are designed to be site specific, and hence to overcome, for example, drug-associated side effects and development of drug resistant tumor cells, these systems are limited by the rate and concentration of the specific enzyme. Since the parent drug is released from the prodrug as a result of its cleavage by the specific enzyme, and hence each such cleavage event release only one molecule of the parent drug, the total amount of the released drug depends on the rate and concentration of the specific enzyme. Moreover, such a mechanism does not enables a simultaneous release of two distinct molecules, which is often time required in various therapeutic applications such as, for example, chemotherapy, chemosensitization, and treatment of nervous systems disorders.

Hence, although the prior art teaches the use of dendrimers in various fields in general and in some biological applications in particular, and further teaches systems that are aimed at a spontaneous and site-specific release of functional moieties such as drugs, the prior art fails to teach the exploitation of the unique structural and biological properties of dendrimers in designing macromolecules that would overcome the present limitations associated with, for example, cancer therapy.

There is thus a widely recognized need for, and it would be highly advantageous to have, dendrimers that are capable of simultaneously release of all their functionality groups as a result of a single event and which are hence devoid of the above limitations.

### SUMMARY OF THE INVENTION

The present invention disclosed the design, synthesis and uses of novel dendrimeric compounds fragmentize into their building blocks in a self-immolative manner upon a single cleavage event, and consequently release all of their tail units.

According to one aspect of the present invention there is provided a self-immolative dendrimer that comprises a cleavable trigger unit, a plurality of tail units and one or more self-immolative chemical linker linking between the trigger unit and the tail units, the trigger unit and the one or more self-immolative chemical linker(s) being such that upon cleavage of the trigger unit, the self-immolative chemical linker(s) self-immolate, thereby releasing the tail units.

According to further features in preferred embodiments of the invention described below, the tail units comprise two or more functional moieties, being the same or different.

According to still further features in the described preferred embodiments the self-immolative dendrimer further comprises one or more self-immolative spacer(s), linking the trigger unit and the self-immolative chemical linker, and/or one or more of the tail units and one or more of chemical linker(s).

According to still further features in the described preferred embodiments, the trigger unit, the spacer(s) and the self-immolative chemical linker(s) being such that upon cleavage of the trigger unit, the self-immolative chemical linker(s) and the spacer(s) self-immolate to thereby release the tail units.

According to still further features in the described preferred embodiments the cleavable trigger unit is selected from the group consisting of a photo-labile trigger unit, a chemically removable trigger, a hydrolysable trigger unit and a biodegradable trigger unit, e.g. an enzymatically cleavable trigger unit.

According to still further features in the described preferred embodiments the functional moieties comprise one or more or two or more therapeutically active agent(s), which are preferably synergistic. Preferably, the therapeutically active agent or agents are selected from the group consisting of an anti-proliferative agent (e.g., a chemotherapeutically active agent), an anti-inflammatory agent, an antibiotic, an anti-viral agent, an anti-hypertensive agent, a chemosensitizing agent and a combination thereof

According to still further features in the described preferred embodiments the functional moieties comprise one or more diagnostic agent(s). The diagnostic agent(s) are preferably selected from the group consisting of a signal generator agent, a single absorber agent and a combination thereof.

According to still further features in the described preferred embodiments the self-immolative chemical linker has a general formula selected from the group consisting of Formula Ia and Formula Ib: 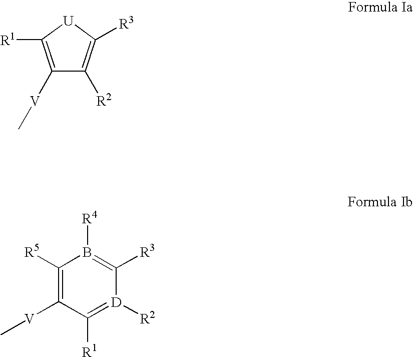
wherein:
V is O, S, PR⁶ or NR⁷;
U is O, S or NR⁸;
B and D are each independently a carbon atom or a nitrogen atom;
R¹, R², R³, R⁴ and R⁵ are each independently 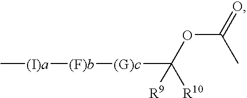
   hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, two or more of R¹, R², R³, R⁴ and R⁵ being connected to one another to form an aromatic or aliphatic cyclic structure;
   whereas:
a, b and c are each independently as integer of 0 to 5; and
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that two or more of R¹, R² and R³ in Formula Ia and of R¹, R², R³, R⁴ and R⁵ in Formula Ib are

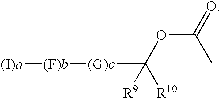

Preferably, the self-immolative chemical linker has the general Formula Ib, and, more preferably, V is O or S; each of B and D is a carbon atom; each of R² and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, two or more of R², R³ and R⁴ being connected to one another to form an aromatic or aliphatic cyclic structure; and each of R¹ and R³ and, optionally, R⁵ is

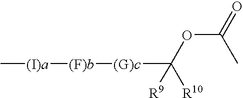

According to still further features in the described preferred embodiments the self-immolative spacer has a general formula selected from the group consisting of Formula IIa, Formula IIb, Formula IIc and Formula IId: 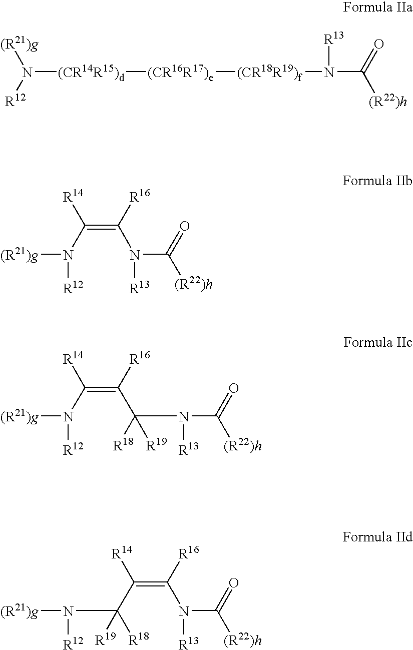
and a combination thereof,
wherein:
d, e, f, g and h are each independently an integer from 0 to 3, provided that d+e+f≧2;
R¹² and R¹³ are each independently hydrogen, alkyl or cycloalkyl;
R¹⁴, R¹⁵, R¹⁶, R^{17,} R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate; and
R²¹ and R²² each independently has a general formula selected from the group consisting of Formula VIIa and Formula VIIb: 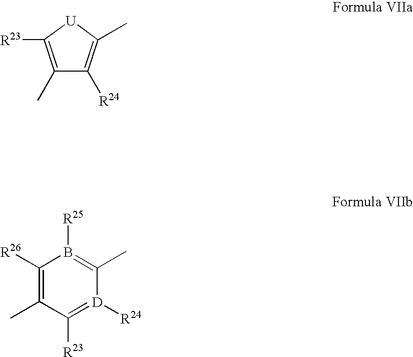
   wherein:
U is O, S or N²⁹;
B and D are each independently a carbon atom or a nitrogen atom;
R²³, R²⁴, R²⁵ and R²⁶ are each independently 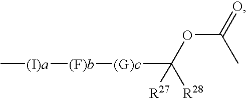
   hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, two or more of R²³, R²⁴, R²⁵ and R²⁶ being connected to one another to form an aromatic or aliphatic cyclic structure;
   whereas:
a, b and c are each independently an integer of 0 to 5;
I, F and G are each independently —R³⁰C═CR³¹— or —C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that two or more of R²³ and R²⁴ in Formula VIIa and of R²³, R²⁴, R³⁵ and R²⁶ in Formula VIIb are

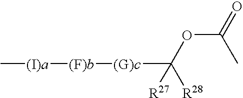

Preferably, the self-immolative spacer has the general Formula IIa.

The self-immolative dendrimer described above if preferably between a first and a tenth generation dendrimer.

Further preferably, the self-immolative dendrimer has between 2 and 5 ramifications in each generation.

In one embodiment, the trigger unit is an enzymatically cleavable trigger unit and the functional moieties comprise one or more therapeutically active agent(s) (e.g. a chemotherapeutic agent) or two or more therapeutically active agents.

In another embodiment, the trigger unit is an enzymatically cleavable trigger unit and the functional moieties comprise one or more diagnostic agent(s) (e.g., a fluorogenic agent).

In still another embodiment, the trigger unit is a photo-labile trigger unit and the functional moieties comprise one or more diagnostic agent(s).

In yet another embodiment, the trigger unit is a hydrolyzable trigger unit and the functional moieties comprise one or more agrochemical(s).

In still another embodiment, the trigger unit is a chemically removable trigger unit and the functional moieties comprise one or more diagnostic agent(s).

According to another aspect of the present invention there is provided a self-immolative dendrimer having a general Formula III:
Q-Ai-Z⁰[(X₀)](Y₀)k]-Z¹[(X₁)I(Y₁)m]- . . . -Zⁿ[(Xn)p(Yn)r]-Zⁿ⁺¹[W] Formula III
wherein:
n is an integer from 0 to 10;
each of i, j, k, l, m, p and r is independently an integer of 0 to 10;
Q is a cleavable trigger unit, as is described hereinabove;
A is a first self-immolative spacer, as is described hereinabove;
Z is an integer of between 2 and 6, representing the ramification number of the dendrimer;
X is a self-immolative chemical linker, as is described hereinabove;
Y is a second self-immolative spacer, as is described hereinabove; and
W is a tail unit,
whereas, when n equals 0, each of l, m, p and r equals 0; and
when n equals 1, each of p and r equals 0.

Preferably, Zⁿ⁺¹ [W] comprise two or more functional moieties, being the same or different, and as is described hereinabove.

Further preferably Z equals 2 or 3 and/or n is an integer of 0 to 10.

According to yet another aspect of the present invention there is provided a pharmaceutical composition, which comprises, as an active ingredient, a self-immolative dendrimer as is described hereinabove and a pharmaceutically acceptable carrier.

The pharmaceutical composition is preferably packaged in a packaging material and identified in print, in or on the packaging material, for use in the treatment of a disease or disorder selected from the group consisting of a proliferative disease or disorder, an inflammatory disease or disorder, a bacterial disease or disorder, a viral disease or disorder and a hypertensive disease or disorder.

Alternatively, the pharmaceutical composition is packaged in a packaging material and identified in print, in or on the packaging material, for use in diagnoses.

According to still another aspect of the present invention there is provided an agricultural composition, comprising, as an active ingredient, a self-immolative dendrimer as is described hereinabove, having an hydrolizable trigger unit and two or more agrochemical tail units, and an agricultural acceptable carrier.

According to an additional aspect of the present invention there is provided a method of treating a disorder or disease selected from the group consisting of a proliferative disease or disorder, an inflammatory disease or disorder, a bacterial disease or disorder, a viral disease or disorder and a hypertensive disease or disorder in a subject in need thereof. The method comprises administering to the subject a therapeutically effective amount of the self-immolative dendrimer as is described hereinabove, which has one or more therapeutically active agent(s) as its tail unit.

According to an additional aspect of the present invention there is provided a method of diagnosis, comprising administering to a subject a therapeutically effective amount of the self-immolative dendrimer as is described hereinabove, which has one or more diagnostic agent(s) as its tail unit.

According to yet an additional aspect of the present invention there is provided a method of determining a concentration of an enzyme. The method comprises contacting, in vitro or in vivo, the enzyme and a self-immolative dendrimer as is described above, which has an enzymatically cleavable trigger unit and one or more diagnostic agents as its tail units.

According to still an additional aspect of the present invention there is provided a method of determining a concentration of a chemical reagent. The method comprises contacting the chemical reagent with a self-immolative dendrimer described above, which has a chemically cleavable trigger unit and one or more diagnostic agents as its tail units.

Further according to the present invention there are provided methods of synthesizing the self-immolative dendrimers described hereinabove.

The present invention successfully addresses the shortcomings of the presently known configurations by providing self-immolative dendrimers that are capable of releasing all of the tail units upon a single yet versatile cleavage event, disregarding the nature of the cleavable unit and the cleavage conditions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b present graphical structures of a first generation (G1) self-immolative dendrimer according to the present invention (FIG. 1a) and a second generation (G2) self-immolative dendrimer according to the present invention (FIG. 1b), each comprises one cleavable trigger unit, one or more self-immolative chemical linker(s) and two and four tail units, respectively;

FIG. 2 is a scheme presenting the release of four tail units from a G2-self-immolative dendrimers, upon cleavage of the trigger unit, according to the present invention;

FIG. 3 is a schematic representation of the self-immolative mechanism of a representative example of a G1-dendrimer according to the present invention (model Compound 1), initiated by a cleavage, which triggers a spontaneous cyclization followed by 1,4-quinone-methide rearrangements to release two tail units (denoted as “reporter”);

FIG. 4 is a scheme presenting a general synthesis of a representative example of a G1-self-immolative dendrimer (model Compound 1) according to the present invention;

FIG. 5 presents the general structures of representative examples of a G2-self-immolative dendrimer (Compound 10) and a third generation (G3)-self-immolative dendrimer (Compound 11) according to the present invention;

FIG. 6 is a scheme presenting a general synthesis of a representative example of a G2-self-immolative dendrimer according to the present invention;

FIG. 7 is a schematic representation of the self-immolative mechanism of a representative example of a G1-dendrimer that carries three tail units;

FIG. 8 is a scheme presenting the synthesis of a representative example of a G1-self-immolative dendrimer of the present invention, which has two pyrene molecules as its tail units and a photo-labile trigger unit (Compound 25);

FIG. 9 is a scheme presenting the synthesis of a representative example of a G2-self-immolative dendrimer of the present invention, which has four pyrene molecules as its tail units and a photo-labile trigger unit (Compound 28);

FIG. 10 presents HPLC chromatograms of a methanolic solution [50 μM] of the G1-self immolative dendrimer Compound 25, before photo irradiation (a) and after irradiation at t=0 (b), t=4 hours (c) and t=11 hours (d);

FIG. 11 presents plots demonstrating the fragmentation (in %) of the amine intermediate Compound 30 (squares) into free aminomethylpyrene molecules 31 (circles), as a function of time, based on HPLC analysis;

FIG. 12 is a plot presenting the natural logarithm of the concentration of the amine intermediate Compound 30 as a function of time, according to the equation:
ln[30]=−k₁t+ln[30]₀;

FIG. 13 presents HPLC chromatograms of a methanolic solution [50 μM] of the G2-self immolative dendrimer Compound 28, before photo irradiation (a) and after irradiation at t=0 (b), t=6 hours (c) and t=20 hours (d);

FIG. 14 presents plots demonstrating the fragmentation (in %) of the amine intermediate Compound 32 (circles) into the amine intermediate Compound 30 (squares) and of the latter into free aminomethylpyrene molecules 31 (triangles), as a function of time, based on HPLC analysis;

FIG. 15 is a plot presenting the natural logarithm of the concentration of the amine intermediate Compound 32 as a function of time, according to the equation:
ln[32]=−k₂t+ln[32]₀;

FIG. 16 presents comparative plots demonstrating the conversion of the amine intermediate Compound 30 (to free aminomethylpyrene molecules 31), according to the experimental data (30, squares) and to mathematical calculations (30*, straight line);

FIG. 17 presents comparative plots demonstrating the release of free aminomethylpyrene molecules 31 from the G2-SID Compound 28 according to the experimental data (triangles) and to mathematical calculations (straight line);

FIG. 18 is a scheme presenting the synthesis of G1, G2 and G3 self-immolative dendrimers having a BOC (chemically removable) trigger unit and 4-nitroaniline tail units (Compounds 33, 34 and 35, respectively);

FIG. 19 presents plots demonstrating the fragmentation (in %) of the amine salt of Compound 34 (circles) into an amine intermediate (squares), and of the latter into free 4-nitroaniline molecules (triangles), as a function of time, based on HPLC analysis;

FIGS. 20a-b present the self-immolative release of 4-nitroaniline molecules from the G3-SID Compound 35, schematically (FIG. 20a) and as plots demonstrating the fragmentation (in %) of the amine salt Compound 36 (diamonds) into the amine intermediate Compounds 37 and 38 (squares), followed by the release of free 4-nitroaniline molecules (triangles), as a function of time, based on HPLC analysis (FIG. 20b);

FIG. 21 is a plot presenting the natural logarithm of the concentration of the G3-SID amine salt, Compound 36, as a function of time, according to the equation:
ln[36]=−k₃t+ln[36]₀;

FIG. 22 is a scheme presenting the release of two drug molecules from a preferred G1-self-immolative dendrimer of the present invention, Compound 39, upon enzymatic cleavage;

FIG. 23 is a scheme presenting a general synthesis of a preferred G1-self-immolative dendrimer of the present invention, having an enzymatic trigger unit and drug functional moieties (Compound 39);

FIG. 24 is a scheme presenting the conversion of the hydroxy anti-cancer drugs camptothecin (47) and etoposide (50) into the amine derivatives (49 and 52, respectively) thereof by coupling thereto the self-immolative spacer N,N-dimethyletylene-diamine.

FIG. 25 presents the chemical structures of a multi-doxorubicin G1-SID prodrug (Compound 53) and a multi-camptothecin G1-SID prodrug (Compound 54) according to the present invention;

FIG. 26 presents the chemical structures of free doxorubicin (DOX—NH₂), the mono-doxorubicin prodrug 55 and the di-doxorubicin G1-SID prodrug of the present invention (Compound 53);

FIGS. 27a-b present comparative plots demonstrating the inhibition activity of the mono-doxorubicin prodrug 55 (FIG. 27a) and the di-doxorubicin G1-SID prodrug 53 (FIG. 27b), alone (denoted as D-M and D-D, respectively, open circles) and in the presence of the catalytic antibody 38C2 (denoted as D-M+38C2 and D-D+38C2, respectively, open squares), compared with the inhibition activity of free doxorubicin (denoted as D, filled circles) and the solvent (filled triangles);

FIG. 28 is a scheme presenting the release of camptothecin (CPT) from the known mono-CPT prodrug 56 and the di-camptothecin G1-SID prodrug of the present invention 54;

FIG. 29 is a bar graph presenting the anti-proliferative activity of the mono-CPT prodrug 56 and the di-camptothecin G1-SID prodrug 56 alone (left bars) and in combination with the catalytic antibody 38C2 (right bars);

FIGS. 30a-b present comparative plots demonstrating the inhibition activity of the mono-camptothecin prodrug 56 (FIG. 30a) and the di-camptothecin G1-SID prodrug 54 (FIG. 30b), alone (denoted as C-M and C-D, respectively, open circles) and in the presence of the catalytic antibody 38C2 (denoted as C-M+38C2 and C-D+38C2, respectively, open squares), compared with the inhibition activity of free camptothecin (denoted as C, filled circles);

FIG. 31 is a scheme presenting the release of 4-nitroaniline molecules from a preferred G2-SID sensor according to the present invention (Compound 57) upon an enzymatic cleavage;

FIG. 32 is a scheme presenting the synthesis of a representative example of a G2-SID enzymatic sensor according to the present invention (Compound 58);

FIG. 33 is a scheme presenting the release of doxorubicin (DOX) and camptothecin (CPT) from a representative example of a heterogenic G1-SID of the present invention (Compound 61); and

FIG. 34 are comparative plots demonstrating the inhibition activity of a combination of the mono-camptothecin prodrug 56 and the mono-doxorubicin prodrug 55 alone (denoted as D-M+C-M, half-filled circles) and in the presence of the catalytic antibody 38C2 (denoted as D-M+C-M+38C2, filled squares) and of the heterodimeric G1-SID 61 alone (denoted as C/D-D, open circles) and in the presence of the catalytic antibody 38C2 (denoted as C/D-D +38C2, open squares).

### DESCRIPTION OF THE PREFERRED EMBODIMENTS

The present invention is of a self-immolative dendrimer which can release all of its tail units upon a single cleavage and can therefore be beneficially used in a variety of biological applications. Specifically, the dendrimers of the present invention can be used, for example, as highly efficient prodrugs which release a plurality of drug molecules upon a single enzymatic cleavage, in various diagnostic applications and as amplifiers of a myriad of reporting signals for measuring a variety of chemical, biochemical and physical activities, such as, but not limited to, enzymatic activity, chemical activity and/or photoirradiation.

The principles and operation of a self-immolative dendrimer, methods of preparing same and its uses according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is described hereinabove, the presently known applications which utilize the high-group functionality of dendrimers for amplifying the effect of the functional tail groups involve the release of each of the functional tail groups upon an associated cleavage event. Therefore, the efficacy of these prior art dendrimers is limited, as they require a plurality of events to achieve substantial amplification of tail units release.

Hence, in a search for more efficient compounds that are able to simultaneously release a plurality of functional moieties, the present inventors have envisioned that by combining the unique structural properties and synthetic routes of dendrimers described hereinabove and technologies that involve self-immolative systems, highly efficient dendrimers could be designed. More specifically, the present inventors have envisioned that by designing dendrimers that include a cleavable unit as the core, a plurality of self-immolative units that extend outwardly therefrom and a plurality of functional group as the tail units, dendrimers that are capable of releasing all of the functional moieties simultaneously, as a reponse to a single event, could be obtained and efficiently utilized in a variety of applications.

As used herein the term. “simultaneously” is used to indicate a multi-cascade response to a single trigger event.

While reducing the present invention to practice, it was found that various dendrimers, designed as described herein, are both synthesizable and are indeed capable of releasing a plurality of functional tail units upon a single cleavage event. More specifically, it was found that subjecting such dendrimers to conditions that prompt cleavage of the core, triggers a sequence of reactions that results in self-immolation of the dendrimer and thus leads to a spontaneous release of all the tail units upon a single event. These novel dendrimers are therefore referred to herein as self-immolative dendrimers.

Hence, each of the self-immolative dendrimers of the present invention comprises a cleavable trigger unit, a plurality of tail units and one or more self-immolative chemical linker linking between the trigger unit and the tail units. The cleavable trigger unit and the self-immolative chemical linkers of the present invention are designed such that upon cleavage of the trigger unit, the chemical linker self-immolates to thereby release all of the tail units.

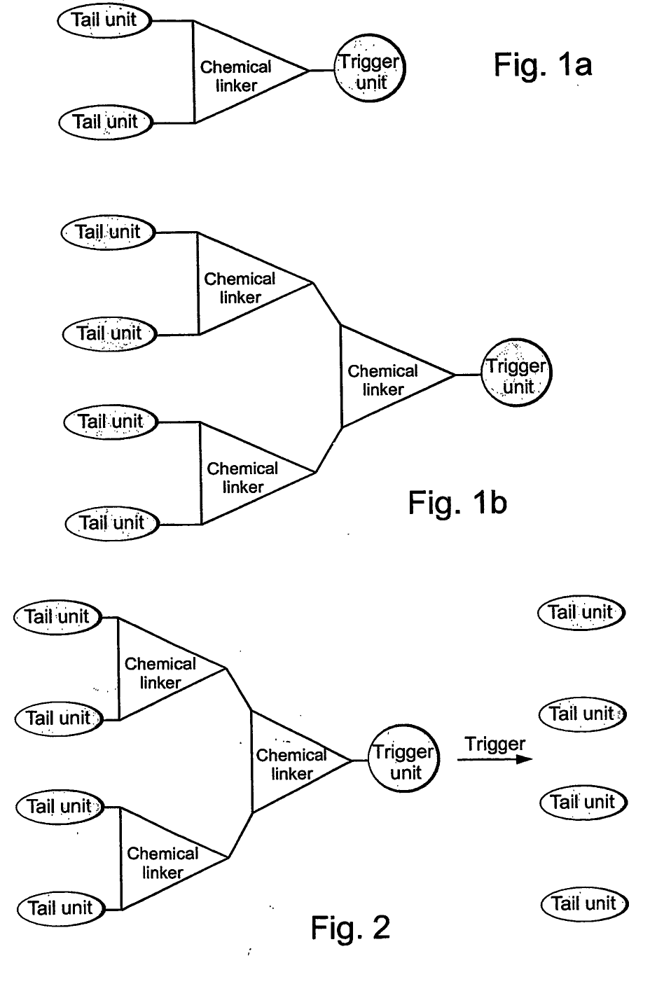

FIGS. 1a and 1b schematically present the structure of representative examples of a G1-self-immolative dendrimer (G1-SID) and a second generation G2-SID according to the present invention, respectively.

As is well known in the art and is used herein throughout, G1, G2 . . . Gn represent the generation number of a dendrimer, such that herein the phrase “a G1-SID” describes a self-immolative dendrimer that comprises a cleavable trigger unit, a chemical linker and two or more tail units, the phrase “a G2-SID” describes a self-immolative dendrimer that comprises a cleavable trigger unit attached to a first chemical linker, which in turn is attached to two or more chemical linkers, each being attached to two or more tail units, and so on.

The self-immolative dendrimers of the present invention are preferably G1-G10 dendrimers, more preferably G2-G6 dendrimers.

As is shown in FIG. 1a, a representative G1-SID according to the present invention comprises a trigger unit and a chemical linker linking the trigger unit and two tail units.

As is shown in FIG. 1b, a representative G2-SID according to the present invention comprises a trigger unit, a chemical linker connecting the trigger unit to another two chemical linkers, each being linked to two tail units, thereby linking the trigger unit to four tail units.

As is shown in FIG. 2, upon intereaction with a trigger that cleaves the trigger unit, the four tail units of the representative G2-SID described in FIG. 1b are released.

It should be noted however, that, as is exemplified in the Examples section that follows, the chemical linker of the present invention can be selected so as to link the trigger unit to more than two tail units, in the case of a G1-SID, or to more than two chemical linkers in the case of a Gn-SID, thus rendering the number of ramifications of the dendrimer of the present invention being between 2 and 5, preferably between 2 and 3.

The self-immolative chemical linker of the dendrimers of the present invention therefore comprises, in accordance with the acceptable dendrimers' chemistry underlines, a multifunctional base unit which enables its linkage to the core unit, in case of a G1-dendrimer, or to two or more other chemical linkers, in case of a Gn-dendrimer where N>1, at one end, and to two or more tail units or to two or more other chemical linkers, respectively, at the other end. The self-immolative chemical linker of the present invention therefore serves, and is also referred to herein interchangeably, as a chemical adaptor.

As is described hereinabove, the self-immolative chemical linker of the present invention is selected such that it undergoes a sequence of self-immolative reactions upon cleavage of the trigger unit.

As is known in the art, self-immolative reactions typically involve electronic cascade self-elimination and therefore self-immolative systems typically include electronic cascade units which self-eliminate through, for example, linear or cyclic 1,4-elimination, 1,6-elimination, etc. Such electronic cascade units are widely described in the art (see, for example, WO 02/083180).

The presently known self-immolative systems are designed to release one end group upon each elimination. In sharp ditinction, the dendrimers of the present invention are designed such that the self-immolative chemical linker undergoes electronic cascade self-elimination to thereby release two or more end groups. Such chemical linkers are preferably based on a multifunctional aromatic unit which can be linked to both the trigger unit and to two or more tail units or other chemical linkers and can further be subjected to electronic cascade self-elimination.

Hence, preferred self-immolative chemical linkers according to the present invention are five- or six-membered aromatic rings that have the following general formulas: 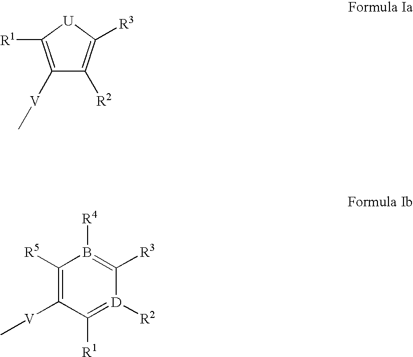
wherein:
V is O, S, PR⁶ or NR⁷;
U is O, S or NR⁸;
B and D are each independently a carbon atom or a nitrogen atom;
R¹, R², R³, R⁴ and R⁵ are each independently 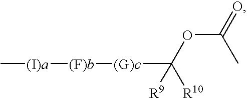
   hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
   whereas:
a, b and c are each independently as integer of 0 to 5; and
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R¹, R² and R³ in Formula Ia and of R¹, R², R³, R⁴ and R⁵ in Formula Ib are

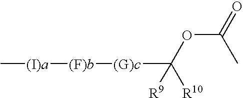

According to these preferred self-immolative chemical linkers of the present invention, in the first generation of the dendrimer, V represents a group that links the chemical linker to the trigger, whereas in the advanced generations (n>1) V represents a group that links the linker to the chemical linkers of a previous generation. As is described hereinabove, V can be an etheric group (—O—), a thioetheric group (—S—), a substituted or non-substituted amino group (—NR⁶—) or a substituted or non-substituted phosphinic group (—PR⁷—).

Further according to these preferred self-immolative chemical linkers of the present invention, the linker is linked to the tail units or to the linkers of the next generation via two or more 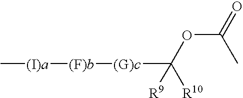
groups. The —(I)a-(F)b-(G)c-unit, if present, is a linear electronic cascade unit that is conjugated to the aromatic system of the basic unit and thereby directly participate in the self-immolative reactions sequence, whereas the carboxy unit —O—(C═O)— enables the release of the linkers/tail units attached thereto via a decarboxylation, which takes place at the end of the self-immolation sequence. The presence of two or more such 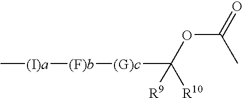
groups as substituents of the aromatic system enables the occurrence of more than one self-immolative reactions sequence at a time. The aromatic system, while being capable to undergo various rearrangements, further enables such occurrence. However, as such rearrangements are more facilitated in a six-membered aromatic ring, the chemical linker of the present invention preferably has the general formula Ib.

Hence, at least two of the rings substituents R¹, R², R³, R⁴ and R⁵ in Formula Ib are 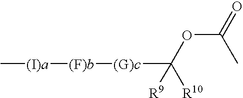
Preferably, at least two of R¹, R³ and R⁵ are

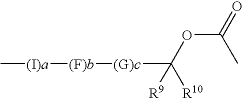

Other ring substituents, as well as the other substituents in Formulas Ia and Ib, R⁶, R⁷, R⁸, R⁹, R¹⁰, R¹¹ and R¹², can be hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,

As used herein, the term “alkyl” refers to a saturated aliphatic hydrocarbon including straight chain and branched chain groups. Preferably, the alkyl group is a medium size alkyl having 1 to 10 carbon atoms. More preferably, it is a lower alkyl having 1 to 6 carbon atoms. Most preferably it is an alkyl having 1 to 4 carbon atoms. Representative examples of an alkyl group are methyl, ethyl, propyl, isopropyl, butyl, tert-butyl, pentyl and hexyl.

As used herein, the term “cycloalkyl” refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene and adamantane.

The term “aryl” refers to an all-carbon monocyclic or fused-ring polycyclic (i.e., rings which share adjacent pairs of carbon atoms) group having a completely conjugated pi-electron system. Examples, without limitation, of aryl groups are phenyl, naphthalenyl and anthracenyl.

The term “phenyl”, according to the present invention can be substituted by one to three substituents or non-substituted. When substituted, the substituent group may be, for example, halogen, alkyl, alkoxy, nitro, cyano, trihalomethyl, alkylamino or monocyclic heteroaryl.

The term “heteroaryl” includes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heterocycloalkyl” refers to a monocyclic or fused ring group having in the ring(s) one or more atoms such as nitrogen, oxygen and sulfur. The rings may also have one or more double bonds. However, the rings do not have a completely conjugated pi-electron system.

As used herein, the term “hydroxy” refers to an —OH group.

The term “thiohydroxy” refers to a —SH group.

The term “alkoxy” refers to both an —O-alkyl and an —O-cycloalkyl group, as defined hereinbelow. Representative examples of alkoxy groups include methoxy, ethoxy, propoxy and tert-butoxy.

The term “thioalkoxy” refers to both a —S-alkyl and a —S-cycloalkyl group, as defined hereinabove.

The term “aryloxy” refers to both an —O-aryl and an —O-heteroaryl group, as defined herein.

A “thioaryloxy” group refers to both an —S-aryl and an —S-heteroaryl group, as defined herein.

As used herein, the term “halo” refers to a fluorine, chlorine, bromine or iodine atom.

The term “trihalomethyl” refers to a —CX₃ group, wherein X is halo as defined herein. A representative example of a trihalomethyl group is a —CF₃ group.

The term “amino” refers to an —NR′R″ group, where R′ and R″ are each independently hydrogen, alkyl or cycloalkyl, as is defmed hereinabove.

The term “cyclic alkylamino” refers to an —NR′R″ group where R′ and R″ form a cycloalkyl.

The term “nitro” refers to a —NO₂ group.

The term “cyano” refers to a —C≡N group.

The term “C-amido” refers to a —C(αO)—NR′R″ group, where R′ and R″ are as described hereinabove.

The term “N-amido” refers to a —NR′—C(═O)—R″, where R′ and R″ are as described hereinabove.

The term “carboxy” refers to a —C(═O)—OH group.

The term “carboxylate” refers to a “—C(═O)—OR′ group, where R′ is as defined hereinabove.

The term “sulfoxy” refers to a “—S(═O)₂OH group.

The term “sulfonate” refers to a “—S(═O)₂OR′ group, where R′ is as defined hereinabove.

An “alkylsulfinyl” group refers to an —S(═O)—R′ group, where R′ is as defined herein.

The term “sulfonyl” refers to an —S(═O)₂—R′ group, where R′ is as defined herein.

The term “sulfixy” refers to an —S(═O)₂—H group.

The term “sulfinate” refers to an —S(═O)—OR′ group, where R′ is as defined hereinabove.

The term “sulfinyl” refers to an —S(═O)R′ group, where R′ is as defined hereinabove.

The term “phosphonooxy” refers to an —O—P(═O)(OH)₂ group.

The term “phosphate” refers to an —O—P(═O)(OR′)(OR″) group, where R′ and R″ are as defined hereinabove.

Alternatively, at least two of R¹, R², R³, R⁴ and R⁵ can be connected to one another, so as to form an aromatic or aliphatic cyclic structure. Thus, for example, the self-immolative linker comprises an aromatic system that include two or more fused rings (e.g., naphthalene or anthracene), or an aromatic ring that is fused to one or more alicyclic rings.

A preferred self-immolative linker according to the present invention has a general Formula Ib, wherein V is O or S, each of B and D is a carbon atom, each of R² and R⁴ is hydrogen or alkyl, a, b and c are all 0 and R⁹ and R¹⁰-are hydrogen or alkyl.

The chemical linker of the present invention is designed to undergo self-immolation upon cleavage of the cleavable trigger unit, which is attached thereto.

As used herein, the phrase “cleavable trigger unit” describes a residue of a compound that can be cleaved by a reaction with the corresponding trigger.

As used herein and is known in the art, the term “residue” describes a major portion of a molecule which is covalently linked to another molecule, herein the chemical linker or the spacer described hereinbelow.

Therefore, the term “trigger” as used herein describes an event that cleaves the trigger unit residue described above from the molecule to which it is attached.

The cleavable trigger of the present invention can be, for example, a photo-labile trigger that is cleaved upon its exposure to light, a chemically removable trigger that is cleaved upon a chemical reaction, such as a hydrolysable trigger that is cleaved upon reacting with a water molecule.

Alternatively or in addition, the cleavable trigger unit according to the present invention can be a biodegradable trigger that is cleaved upon a biological reaction with the appropriate biological trigger. Preferred biological triggers according to the present invention are enzymes, whereas the trigger units are the corresponding enzymatic substrates.

As the dendrimers of the present invention are highly advantageous as being capable of efficiently releasing a plurality of tail units, the tail units preferably include two or more functional moieties that can be simultaneously released.

As used herein, the phrase “functional moieties” includes a residue, as this term is defined hereinabove, of a molecule that exerts certain functionality. Representative examples of functional moieties that can be efficiently utilized by the present invention include, without limitation, therapeutically active agents, diagnostic agents; reporters and agrochemicals, as is detailed hereinbelow.

The two or more functional moieties in the SID of the present invention can be the same or different. In cases where the functional moieties are the same, the SIDs of the present invention provides for substantial enhancement of the functionality of the moieties. In cases where the functional moieties are different one from the other, the SIDs of the present invention provides for simultaneous release of two active agents and can therefore be specifically advantageous in cases where the different moieties are synergistic.

Representative examples of therapeutically active agents that can be efficiently incorporated as tail units in the SIDs of the present invention include, without limitation, anti-proliferative agents, anti-inflammatory agents, antibiotics, anti-viral agents, anti-hypertensive agents, chemosensitizing agents and any combination thereof.

Non-limiting examples of anti-inflammatory agents useful in the context of the present invention include methyl salicylate, aspirin, ibuprofen, and naproxen, and derivatives thereof.

Non-limiting examples of antiviral agents useful in the context of the present invention include famciclovir, valacyclovir and acyclovir, and derivatives thereof.

Non-limiting examples of antibiotics include penicillin-V, azlocillin, and tetracyclines, and derivatives thereof.

As is discussed hereinabove, utilizing dendrimers as anti-proliferative prodrugs is highly beneficial due to the EPR effect. Hence, preferred therapeutically active agents according to the present invention include anti-proliferative agents such as chemotherapeutic agents.

Non-limiting examples of chemotherapeutic agents that can be efficiently incorporated as tail units in the SIDs of the present invention include amino containing chemotherapeutic agents such as daunorubicin, doxorubicin, N-(5,5-diacetoxypentyl)doxorubicin, anthracycline, mitomycin C, mitomycin A, 9-amino camptothecin, aminopertin, antinomycin, N⁸-acetyl spermidine, 1-(2-chloroethyl)-1,2-dimethanesulfonyl hydrazine, bleomycin, tallysomucin, and derivatives thereof; hydroxy containing chemotherapeutic agents such as etoposide, camptothecin, irinotecaan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine and vinblastine, and derivatives thereof, sulfhydril containing chemotherapeutic agents and carboxyl containing chemotherapeutic agents.

Other therapeutically active agents that can be beneficially incorporated in the SIDs of the present invention include, for example, antihistamines, anesthetics, analgesics, anti-fungal agents, vitamins and anti-infectious agents.

As is discussed hereinabove, the SIDs of the present invention can advantageously include different functional moieties, which are preferably synergistic. Hence, the functional moieties of the SIDs of the present invention can include, for example, any combination of the therapeutic agents described hereinabove, which would result in synergism. Representative examples of such synergism include a combination of chemotherapeutic agents, or a combination of a chemotherapeutic agent and a chemosensitizing agent. Other combinations are also understood to be synergistic.

Representative examples of diagnostic agents that can be beneficially incorporated in the SIDs of the present invention include, without limitation, signal generator agents and signal absorber agents.

As used herein, the phrase “signal generator agent” includes any agent that results in a detectable and measurable perturbation of the system due to its presence. In other words, a signal generator agent is an entity which emits a detectable amount of energy in the form of electromagnetic radiation (such as X-rays, ultraviolet (UV) radiation, infrared (IR) radiation and the like) or matter, and includes, for example, phosphorescent and fluorescent (fluorogenic) entities, gamma and X-ray emitters, (such as neutrons, positrons, β-particles, α-particles, and the like), radionuclides, and nucleotides, toxins or drugs labeled with one or more of any of the above, and paramagnetic or magnetic entities.

As used herein, the phrase “signal absorber agent” describes an entity which absorbs a detectable amount of energy in the form of electromagnetic radiation or matter. Representative examples of signal absorber agents include, without limitation, dyes, contrast agents, electron beam specifies, aromatic UV absorber, and boron (which absorbs neutrons).

Representative examples of agrochemicals that can be beneficially incorporated as tail units in the SIDs of the present invention include, without limitation, fertilizers, such as acid phosphates and sulfates; insecticides such as chlorinated hydrocarbons (such as p-dichlorobenzene), imidazoles, and pyrethrins, including natural pyrethrins; herbicides, such as carbamates, derivatives of phenol and derivatives of urea; and pheromones.

According to a preferred embodiment of the present invention, the self-immolative dendrimers of the present invention further comprise a self-immolative spacer. As is well known in the art, the term “spacer” describes a residue, as is defined hereinabove, of a non-functional molecule, which is incorporated in a compound in order to facilitate its function and/or synthesis.

The spacer of the present invention may link the trigger unit and/or one or more functional moieties to the chemical linker.

Incorporation of a self-immolative spacer between the chemical linker and the trigger unit provides for and determines the distance therebetween. Such a distance is oftentimes required to facilitate the cleavage of the trigger unit by rendering the trigger unhindered and non-rigid and thus exposed and susceptible to intereact with the trigger.

Incorporation of a self-immolative spacer between a functional moiety and the chemical linker is typically performed so as to facilitate the incorporation of a tail unit into the SID in terms of, for example, chemical compatibility and/or stearic considerations.

Being selected as self-immolative, the spacer of the present invention participates in the self-immolative reactions sequence of the SIDs of the present invention.

Preferred self-immolative spacers according to the present invention have a general formula selected from Formulas IIa, IIb, IIc and IId below: 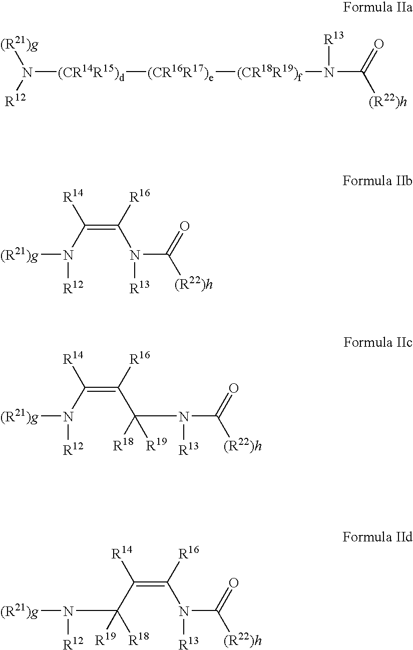
and a combination thereof,
wherein:
d, e, f, g and h are each independently an integer from 0 to 3, provided that d+e+f≧2;
R¹² and R¹³ are each independently hydrogen, alkyl or cycloalkyl;
R¹⁴, R¹⁵, R¹⁶, R¹⁷, R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate;
R²¹ and R²² each independently has a general formula selected from the group consisting of Formula VIIa and Formula VIIb: 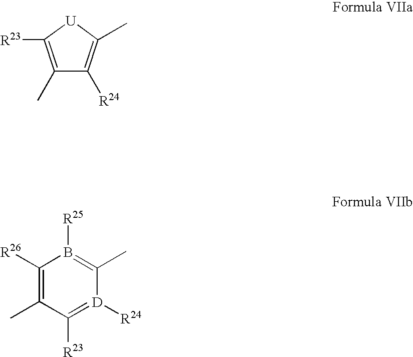
   wherein:
U is O, S or NR²⁹;
B and D are each independently a carbon atom or a nitrogen atom;
R²³, R²⁴, R²⁵ and R²⁶ are each independently 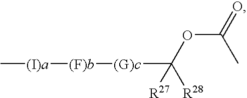
   hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, as these terms are defined hereinabove, or alternatively, at least two of R²³, R²⁴, R²⁵ and R²⁶ being connected to one another to form an aromatic or aliphatic cyclic structure;
   whereas:
a, b and c are each independently as integer of 0 to 5; and
I, F and G are each independently —R³⁰C═CR³¹— or —C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, as these terms are described hereinabove, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, as these terms are defined hereinabove,
provided that at least two of R²³ and R²⁴ in Formula VIIa and of R²³, R²⁴, R³⁵ and R²⁶ in Formula VIIb are:

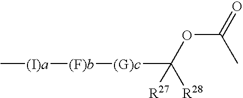

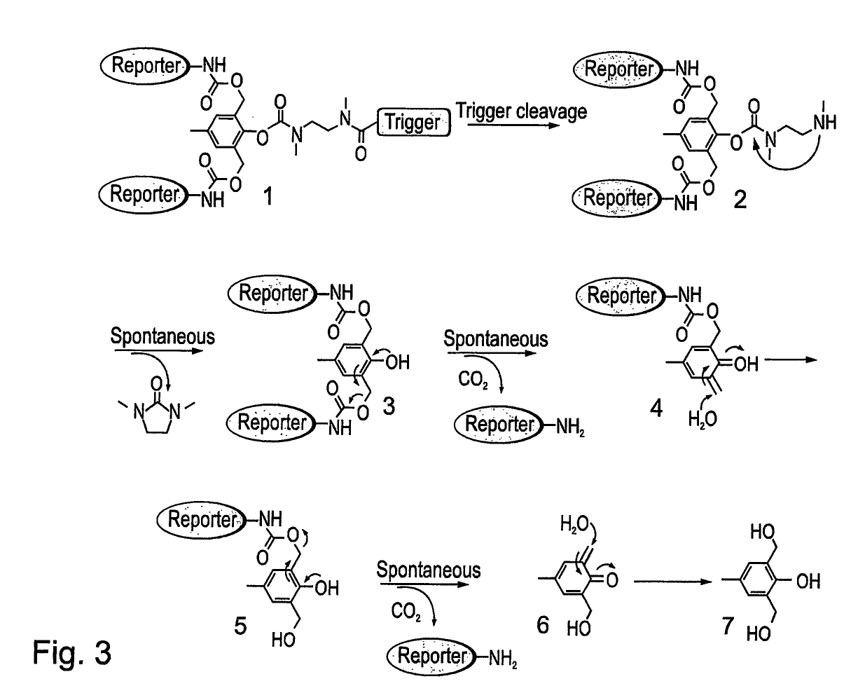

The spacers presented by Formulas IIa, IIb, IIc and IId therefore belong to the known ω-amino aminocarbonyl cyclization spacers, which undergo self-elimination via a cyclization process (as is exemplified, for example, in FIGS. 3, 7 and 22), so as to form urea derivatives. Such self-immolative spacers are therefore specifically advantageous in SIDs that are intended for biological applications, as they result in biocompatible side products such as urea. Furthermore, by being terminated with an amine group, such spacers enable the formation of amide bonds, which, as is exemplified in the Examples section below, are preferable bonds in various embodiments of the present invention.

As is described hereinabove, the self-immolative spacer of the present invention can also comprise any combination of the spacers presented in Formulas IIa, IIb, IIc and IId, and, as is defined hereinabove, may further be interrupted with units that self-immolate via the electronic cascade self-elimination described hereinabove.

The chemical characteristics and the length of the self-immolative spacer can be tailored according to specific requirements, needs and/or preferences. For example, in cases where the tail units are large, bulky molecules and the reaction of the trigger unit and the trigger requires unhindered trigger unit (as in the case of enzymatic cleavage), a long self-immolative spacer may be incorporated in the SID, so as to avoid stearic hindrance of the trigger unit and hence, the selected spacer would comprise several, same or different, self-immolative spacer units. Also, in cases where the tail unit does not have a functional group that enables its attachment to the selected chemical linker, an appropriate spacer that can “divert” the functional group of the tail unit, can be incorporated.

Hence, the self-immolative dendrimers of the present invention are comprised of a cleavable trigger unit, one or more self-immolative chemical linkers, a plurality of tail units and optionally one or more self-immolative spacers, all are attached one to the other in accordance with the unique dendrimeric structure.

The SIDs of the present invention can therefore be presented by Formula III, as follows:
Q-Ai-Z⁰[(X₀)j(Y₀)k]-Z¹[(X₁)l(Y₁)m]- . . . -Zⁿ[(Xn)p(Yn)r]-Zⁿ⁺¹[W] Formula III
wherein:
n is an integer from 0 to 20;
each of i, j, k, l, m, p and r is independently an integer of 0 to 10;
Q is a cleavable trigger unit, as is defined hereinabove;
A is a first self-immolative spacer, as is defined hereinabove;
Z is an integer of between 2 and 6, representing the ramification number of the dendrimer;
X is a self-immolative chemical linker, as is described hereinabove;
Y is a second self-immolative spacer, and
W is a tail unit,
whereas, when n equals 0, each of l, m, p and r equals 0; and
when n equals 1, each of p and r equals 0.

As has already been mentioned hereinabove, the ramification number of the SIDs of the present invention, represented by Z in Formula III is preferably 2 or 3, yet can also be 4 or 5. The tail units W are preferably functional moieties, as is defined and described hereinabove.

The number of generations of the SIDs, n, is preferably 1-10, more preferably, 2-6.

As is demonstrated in the Examples section that follows, the SIDs of the present invention can be easily designed, by selecting the appropriate linkages between the components, to be completely stable prior to contacting the trigger. The SIDs may be further designed to self-immolate in an aqueous medium, a feature that is highly advantageous in some of the applications that utilize these SIDs.

As is exemplified in the Examples section that follows, while reducing the present invention to practice, self-immolative dendrimers as described hereinabove, having various trigger units and various functional moieties as tail units have been synthesized and successfully tested for their capability to simultaneously release the tail units, thus demonstrating the versatility of the self-immolative dendrimers of the present invention, as is described hereinbelow.

In one example, an SID according to the present invention comprises an enzymatically cleavable trigger unit and one or more therapeutically active agents as tail units, and may therefore serve as a highly efficient prodrug, as is demonstrated hereinbelow.

In another example, an SID of the present invention comprises an enzymatically cleavable trigger unit, a chemically removable trigger unit or a photo-labile trigger unit and a plurality of diagnostic agent molecules as tail units, thus providing an efficient diagnostic tool, as is detailed and demonstrated hereinbelow.

In another example, an SID of the present invention comprises a hydrolysable trigger unit and one or more agrochemical agents as tail units and may therefore serve as an efficient pesticide or any other beneficial agricultural composition.

Hence, according to another aspect of the present invention, there is provided a method of treating a disorder or disease, such as, but not limited to, a proliferative disease or disorder, an inflammatory disease or disorder, a bacterial disease or disorder, a viral disease or disorder and a hypertensive disease or disorder in a subject in need thereof. The method, according to this aspect of the present invention is effected by administering to the subject a therapeutically effective amount of a self-immolative dendrimer that comprises one or more therapeutically active agents as tail units. Preferably, the SID utilized in this method further comprises an enzymatically cleavable trigger unit.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease, substantially ameliorating clinical symptoms of a disease or substantially preventing the appearance of clinical symptoms of a disease.

The term “administering” as used herein refers to a method for bringing a SID of the present invention into an area or a site in the subject that is impaired by the disorder or disease.

The term “therapeutically effective amount” refers to that amount of the SID being administered which will relieve to some extent one or more of the symptoms of the disorder or disease being treated.

This method of the present invention is highly efficient as compared with the presently known corresponding methods, since the dendrimers of the present invention enable to select the suitable components, e.g., trigger unit and tail units, such that the method provides for: (i) targeted delivery of the SID to the specific site by selecting a trigger unit that is cleaved by an enzyme secreted or expressed at this site; (ii) simultaneous release of the therapeutically active agents, such that enhanced concentration of the agents is applied to the targeted site upon one administration and one cleavage event; and (iii) simultaneous release of synergistic therapeutically active agents, if preferred.

According to yet another aspect of the present invention, there is provided a method of performing a diagnosis, which is effected by administrating to a subject a diagnostically effective amount of a SID of the present invention having an enzymatically cleavable trigger unit and one or more diagnostic agents as its tail units.

The diagnostic agent can be a signal generator agent and/or a signal absorber agent, as these phrases are defmed hereinabove.

The phrase “a diagnostically effective amount” includes an amount of the agent that provides for a detectable and measurable amount of the energy emitted or absorbed thereby.

The method according to this aspect of the present invention can therefore be utilized to perform diagnoses such as, for example, radioimaging, nuclear imaging, X-ray, diagnoses that involve contrasts agents and the like, using the suitable tail units, as is detailed hereinabove.

This method is highly advantageous as it provides for substantial amplification of the signal generated or absorbed by the diagnostic agent upon a single administration thereof, and may further provides for targeted delivery of the diagnostic agent to the relevant site or organ, as is described hereinabove.

A SID of the present invention which comprises an enzymatically cleavable trigger and a diagnostic agent can further be utilized to determine enzymatic concentrations. Hence, according to yet another aspect of the present invention, there is provided a method of determining a concentration of an enzyme, which is effected by contacting the enzyme with such a self-immolative dendrimer and monitoring the rate of immolation induced by enzymatic trigger.

This method can be effected in vitro, to thereby determine a concentration of an enzyme in, for example, cells cultures or samples. The diagnostic agent in this case can be, for example, a fluorogenic agent that fluoresces or quenches upon release, such that the enzyme concentration is determined by a simple fluorescence measurement.

Alternatively, this method can be effected in vivo, to thereby determine enzyme concentration in certain organs or tissues and hence serves also as a diagnostic method.

Similarly, a SID of the present invention which has a chemically removable trigger unit and one or more diagnostic agents as its tail units may serve to determine a concentration of a chemical reagent.

Hence, according to another aspect of the present invention there is provided a method of determining a concentration of a chemical reagent, which method is effected by contacting the tested chemical reagent with a self-immolative dendrimer as described hereinabove, which has a trigger unit that is cleaved by this chemical reagent.

Some of the methods described above involve administration of the SIDs of the present invention to a subject. The SID used in these methods can be administered per se, or formulated in a pharmaceutical composition.

Hence, according to still another aspect of the present invention, there are provided pharmaceutical compositions, which comprise any of the SIDs described above and a pharmaceutically acceptable carrier.

Depending on the selected components of the SIDs, the pharmaceutical compositions of the present invention are packaged in a packaging material and identified in print, in or on the packaging material, for use in either use treatment of a disease or disorder selected from the group consisting of a proliferative disease or disorder, an inflammatory disease or disorder, a bacterial disease or disorder, a viral disease or disorder and a hypertensive disease or disorder or for diagnosis, as described hereinabove.

As used herein a “pharmaceutical composition” refers to a preparation of one or more of the SIDs described herein, with other chemical components such as pharmaceutically suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Routes of administration: Suitable routes of administration may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections.

Composition/formulation: Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the SIDs into preparations which, can be used pharmaceutically. Proper formulation is dependent upon the route of administration chosen.

For injection, the SIDs of the invention may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

For oral administration, the SIDs can be formulated readily by combining the SIDs with pharmaceutically acceptable carriers well known in the art. Such carriers enable the SIDs of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active SID doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the SIDs may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the SIDs for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the SID and a suitable powder base such as lactose or starch.

The SIDs described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the SI]) preparation in water-soluble form. Additionally, suspensions of the SIDs may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the SIDs to allow for the preparation of highly concentrated-solutions.

Alternatively, the SID may be in powder form for constitution with a suitable vehicle, e.g., sterile, pyrogen-free water, before use.

The SIDs of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Dosage: Pharmaceutical compositions suitable for use in context of the present invention include compositions wherein the active ingredients are contained in an amount effective to achieve the intended purpose. More specifically, a therapeutically effective amount means an amount of SID effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any SID used in the methods of the invention, the therapeutically effective amount or dose can be estimated initially from activity assays in animals. For example, a dose can be formulated in animal models to achieve a circulating concentration range that includes the IC₅₀ as determined by activity assays (e.g., the concentration of the test SID, which achieves a half-maximal inhibition of cells). Such information can be used to more accurately determine useful doses in humans.

Toxicity and therapeutic efficacy of the SIDs described herein can be determined by standard pharmaceutical procedures in experimental animals, e.g., by determining the IC₅₀ and the LD₅₀ (lethal dose causing death in 50% of the tested animals) for a subject SID. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to provide plasma levels of the active moiety which are sufficient to maintain the desired effects, termed the minimal effective concentration (MEC). The MEC will vary for each preparation, but can be estimated from in vitro data; e.g., the concentration necessary to achieve 50-90% inhibition of proliferation may be ascertained using the assays described herein. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Packaging: Compositions of the present invention may, if desired, be presented in a pack or dispenser device, such as an FDA approved kit, which may contain one or more unit dosage forms containing the active ingredient The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a SID of the invention formulated in a compatible pharmaceutical carrier may also be prepared, placed in an appropriate container, and labeled for treatment of an indicated condition or diagnosis.

As the SIDs of the present invention may further comprise agrochemicals as the tail units, there is also provided herein an agricultural composition, which comprises an SID that have hydrolysable trigger unit and one or more agrochemical as its tail units, and an agricultural acceptable carrier. Such an agricultural composition is highly beneficial since (i) prior to its contact with water the composition is stable and hence non-toxic; (ii) it provides for rapid and efficient release of the agrochemicals upon contacting with water; and (iii) it enables administration of two or more synergistic agrochemicals simultaneously and in a single composition.

Further according to the present invention there are provided methods of synthesizing the SIDs of the present invention.

In one embodiment of this aspect of the present invention, there is provided a methods of synthesizing a first generation self-immolative dendrimer.

The method is effected by providing a first compound having a self-immolative chemical linker being linked to at least two tail units and to a first reactive group; and coupling this compound with a cleavable trigger unit. In cases where the SID further comprises a self-immolative spacer that links the trigger unit and the chemical linker, the method is further effected by coupling the first compound with the spacer, prior to the trigger unit. Preferably, each of the tail units in the first compound is linked to the chemical linker via a carbamate bond.

As used herein, the phrase “carbamate bond” describes a —O—C(═O)—NR′— bond, where R′ is hydrogen, alkyl, or cycloalkyl, as is defined hereinabove.

Such a linkage is advantageous as it provides for a stable linkage between the tail group and the chemical linker prior to initiation of the self-immolation process by the trigger, and can be simply obtained by reacting a preferred chemical linker according to the present invention, which terminates with a 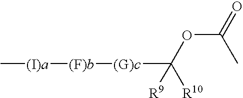
group, attached to a leaving group, with a tail unit that is derived from a compound that has at least one free amino group.

Hence, preferably, the tail units are derived from one or more second compounds that have a free amino group.

However, as is discussed hereinabove and is further detailed hereinbelow in the Examples section, in cases where the these second compounds do not have free amino group or in cases where it is preferable to link the tail unit to the chemical linker via a spacer, the method of synthesizing the G1-SID further comprises attaching a self-immolative spacer to the compound(s) from which the tail units are derived, which is referred to herein as a third compound, and thereafter coupling two or more equivalents of these compounds to the chemical linker in the first compound.

The reactive group in the first compound of this aspect of the present invention is preferably a hydroxyl, a thiol or an amine group, which enables simple and easy coupling of the first compound with the trigger unit. The reactive group is preferably protected prior to this coupling, by a protecting group, which is easily removed. Any of the protecting groups known in the art can be used herein.

Hence, a preferred first compound according to this aspect of the present invention has a general formula IVa or IVb: 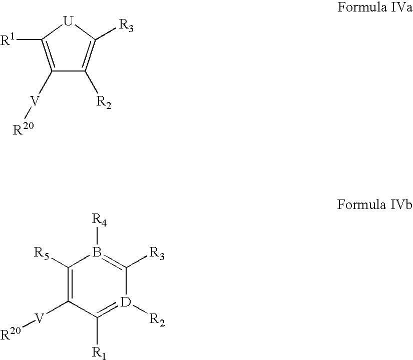
wherein:
V is O, S, PR⁶ or NR⁷;
U is O, S or NR⁸;
B and D are each independently a carbon atom or a nitrogen atom;
R¹, R², R³, R⁴ and R⁵ are each independently 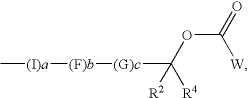
   hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, as these terms are defined hereinabove, or alternatively, at least two of R¹, R², R³, R⁴ and R⁵ being connected to one another to form an aromatic or aliphatic cyclic structure;
   whereas:
a, b and c are each independently as integer of 0 to 5;
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, as these terms are defined hereinabove, or, alternatively, R¹¹ and R¹² being connected to one another to form an aromatic or aliphatic cyclic structure; and
W is one of said at least two tail unit;
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate; and
R²⁰ is hydrogen, alkyl or cycloalkyl,
provided that at least two of R¹, R² and R³ in Formula Ia and of R¹, R², R³, R⁴ and R⁵ in Formula Ib are said

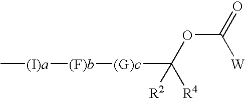

Based on this synthetic approach, Nth generation self-immolative dendrimers where N is an integer greater than 1 (e.g., 2, 3, 4 and up to 10) can be similarly synthesized. The building block of such a Gn-SID is a multifunctional compound derived from the self-immolative chemical linker of the present invention, described hereinabove, which has three or more reactive groups that enable its coupling to other chemical linkers or to the tail units.

Hence, in another embodiment of this aspect of the present invention there is provided a method of synthesizing a Nth generation self-immolative dendrimer. The method of this aspect of the present invention is effected by first providing a (N−1)th generation self-immolative dendrimer including a first self-immolative chemical linker being linked to a first reactive group, (N−1) (N−1)th self-immolative chemical linkers each being linked to at least two second reactive groups, and a plurality of self-immolative chemical linkers linking therebetween, and a Nth compound having a Nth self-immolative chemical linker being linked to a (N+1)th reactive group and to at least two (N+2)th reactive groups, and thereafter coupling at least 2(N−1) equivalents of the Nth compound to the (N−1)th generation self-immolative dendrimer, and coupling the resulting Nth generation self-immolative dendrimer, terminating with at least 2N (N+2)th reactive groups with at least 2N equivalents of at least one (N+1)th compound, to thereby provide said Nth generation self-immolative dendrimer having 2N tail units and a first reactive group as its core.

In one particular of this embodiment, the first reactive group is the cleavable trigger unit. In another, preferable, particular of this embodiment, the first reactive group is hydroxyl, thiol or amine group, which is further preferably protected with a protecting group along the synthesis and is reacted with the cleavable trigger unit after removal of the protecting group, either before or after the coupling of the Nth compound to the (N−1)th generation self-immolative dendrimer.

In still another particulars of this embodiment, in cases where the cleavable trigger unit and/or the tail units are linked to a chemical linker via self-immolative spacers, the method further comprises coupling the first chemical linker or the first reactive group to the self-immolative spacer, prior to the coupling with the trigger unit, and/or coupling the compound from which the tail units are derived from, with the spacer, prior to its coupling with the reactive groups of the (N−1)chemical linker, respectively.

As the preferred linkage between the chemical linkers of each generation and between the chemical linkers of the Nth generation and the tail units are carbamate bonds, preferably the second and the (N+2)th reactive groups comprises a carbonate functional group, whereas the (N+1)th compound comprises a free amino group.

Additional preferred embodiments relating to the synthesis methods described hereinabove are detailed and exemplified in the Examples section that follows.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Design and General Synthesis of a G1-Self-Immolative Dendrimer

As a representative example of a G1-self-immolative dendrimer according to the present invention, a model of such a G1-dendrimer, which is based on the commercially available, tri-functional compound 2,6-bishydroxymethyl-p-cresol, was designed. As is shown in FIG. 3, the model, Compound 1, includes two tail units that are attached through a carbamate linkage to the two benzyl alcohols groups of the basic unit 2,6-bishydroxymethyl-p-cresol (Compound 7), and a trigger unit that is linked to the phenol functionality of the basic unit 2,6-bishydroxymethyl-p-cresol through a short N,N-dimethyletylenediamine spacer. As is farther shown in FIG. 3, according to this model, a cleavage of the trigger unit (denoted as “trigger” in FIG. 3) initiates self-immolative reactions sequence of the cleaved compound, the amine intermediate Compound 2, starting with spontaneous cyclization to form an N,N-dimethylurea derivative and the phenolic Compound 3. The generated phenol 3 goes through 1,4-quinone methide rearrangement, which is followed by spontaneous decarboxylation, to thereby liberate one of the tail units (denoted as “reporter” in FIG. 3). The quinone-methide species 4 is rapidly trapped by a water molecule (from the reaction solvent) to form the phenol Compound 5, which again undergoes a 1,4-quinone-methide rearrangement, to thereby liberate the second tail unit (denoted as “reporter” in FIG. 3). The generated quinone-methide species 6 is trapped again by a water molecule, to give the basic unit 2,6-bishydroxymethyl-p-cresol, Compound 7. Such a double 1,4-quinone-methide rearrangement has not been known heretofore in aromatic systems as the one described herein.

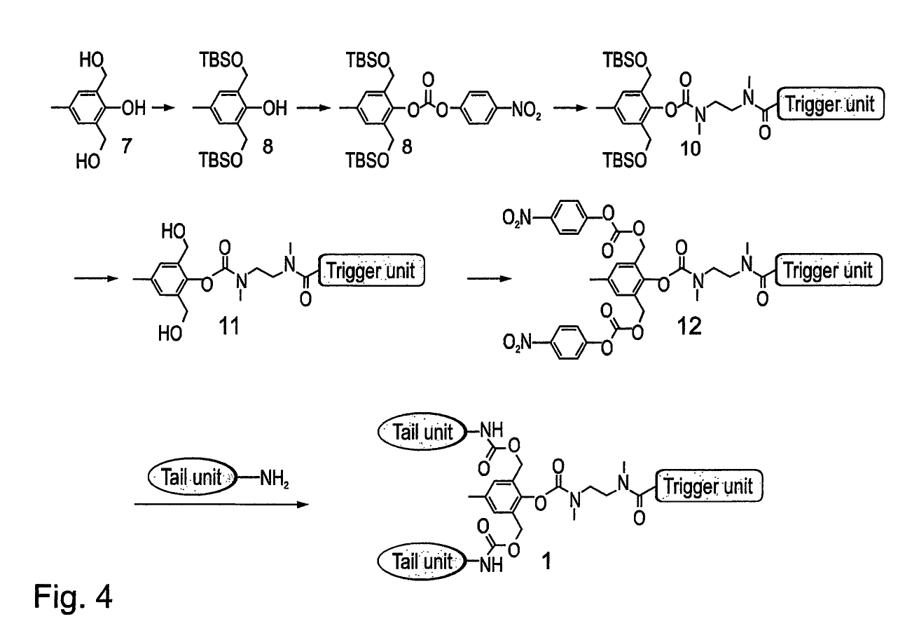

The general synthesis of the G1-dendrimer model described above (Compound 1) is depicted in FIG. 4. 2,6-Bishydroxymethyl-p-cresol (Compound 7) is selectively protected with two t-butyldimethylsilyl (TBS) groups, to give the protected phenol 8, which is then reacted with p-nitrophenyl-chloroformate, to give the corresponding carbonate Compound 9. Compound 9 is thereafter reacted with the spacer unit N,N-dimethyletylene-diamine, linked to a trigger unit, to thereby form the model Compound 10. Deprotection of the TBS groups under mild acidic conditions gives the dibenzyl-alcohol model Compound 11, which is further reacted with two equivalents of p-nitrophenyl-chloroformate, to thereby form the di-carbonate model Compound 12. Reaction of two tail unit-amine molecules with the di-carbonate groups of Compound 12 gives the final model Compound 1.

### Example 2

### Design and General Synthesis of G2- and G3-Self-Immolative Dendrimers

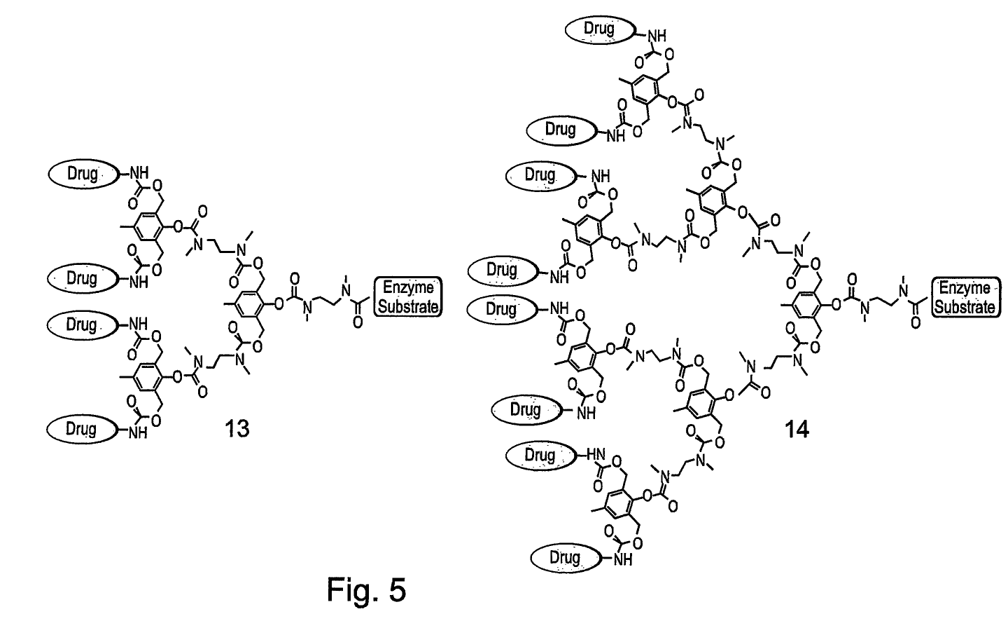

Based on the G1-self-immolative dendrimer model described above, models of higher generations of such dendrimers, e.g., G2- and G3-self-immolative dendrimers, have been designed. Representative examples of a G2-self-immolative dendrimer (Compound 13), and a G3-self-immolative dendrimer (Compound 14), according to the present invention, are presented in FIG. 5. As is shown in FIG. 5, a G2-self-immolative dendrimer is obtained by linking two identical units of G1-dendrimers to the hydroxybenzyl functionalities of the basic cresol (see, Compound 7, FIG. 4) through a double carbamate attachment, using N,N-dimethyletylenediamine as a self-immolative spacer. A G3-self-immolative dendrimer, Compound 14, can be similarly obtained by linking two G2-dendrimers to the hydroxybenzyl groups of the basic cresol. These models have been designed such that the selected linkage between the dendrimeric units (e.g., the N,N-dimethyletylenediamine spacer) affords the requested self-immolative reaction sequence that will lead to the release of the tail units (denoted as “drug” in FIG. 5), whereas the carbamate linkage between the units is highly stable until the trigger unit is cleaved and the self-immolative reaction sequence is initiated.

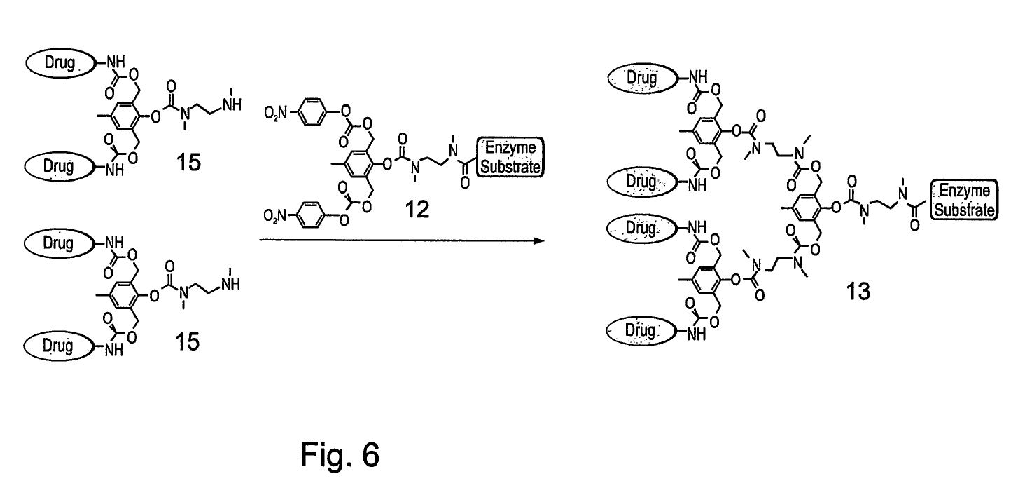

FIG. 6 depicts the general synthesis strategy of a G2-self-immolative dendrimer. Two equivalents of the G1-dendrimer described in Example 1 (Compound 1, FIG. 4) are deprotected, so as to form the amine-salt derivatives 15, which are further reacted with the di-p-nitrophenyl-carbonate intermediate (Compound 9, FIG. 4), to give the desired G2-dendrimer 13.

### Example 3

### Design and General Synthesis of a G1-Self-Immolative Dendrimeric Unit Carrying Three Tail Units

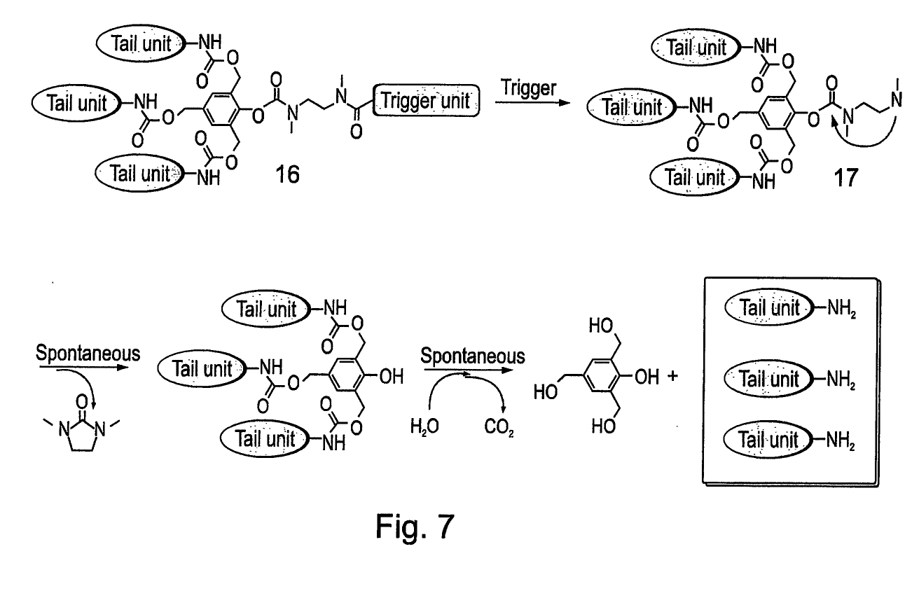

Based of the G1-dendrimer model described hereinabove in Example 1, a model of a G1-dendrimeric compound that carries up to three tail units was also designed. The principle of designing such a compound is based on adding an additional hydroxybenzyl substitution at the para position to the phenolic oxygen of the basic cresol, which enables the additional attachment of a tail unit through a carbamate linkage. As is shown in FIG. 7, the thus formed dendrimeric Compound 16, releases the three tail units upon cleavage of the trigger unit, to give intermediate 17, and a spontaneous cyclization, to give intermediate 18, followed by double 1,4- and one 1,6-quinone methide rearrangements.

The synthesis of Compound 16 is performed similarly to the synthesis of Compound 1 (presented in FIG. 4), using the tetra-functional starting molecule 2,4,6-trishydroxymethyl-phenol, instead of Compound 7. As 2,4,6-trishydroxymethyl-phenol is not commercially available, it can be synthesized by allylic bromination of the TBS-diether derivative of 2,6-bishydroxymethyl-p-cresol (compound 8, FIG. 4), followed by an SN2 type substitution of the bromide with hydroxy group.

The multi-tail units dendrimeric compound described herein is highly advantageous as it enables the synthesis of self-immolative dendrimers with higher number of branching arms, carrying more tail units (e.g., drugs), which could all be released upon a single event.

### Example 4

### Synthesis of G1- and G2-Self-Immolative Dendrimers with a Photo-Labile Unit

General Methods:

Thin layer chromatography (TLC) was performed with silica gel plates Merck 60 F₂₅₄. The compounds were visualized by irradiation with UV light and/or by treatment with a solution of 25 grams phosphomolybdic acid, 10 grams Ce(SO₄)₂.H₂O, 60 ml concentrated H₂SO₄ and 940 ml H2O, followed by heating and/or by staining with a solution of 12 grams 2,4dinitrophenylhydrazine in 60 ml concentrated H₂SO₄, 80 ml H2O and 200 ml 95% EtOH, followed by heating.

Flash chromatography (FC) was performed with silica gel Merck 60 (particle size 0.040-0.063 mm).

¹H-NMR spectra were measured using Bruker Avanee operated at 200 MHz. The chemical shifts are expressed in δ relative to TMS (δ=0 ppm) and the coupling constants J in Hz. The spectra were recorded in CDCl₃ as solvent at room temperature unless stated otherwise. All general reagents, including salts and solvents, were purchased from Aldrich (Milwaukee, Minn.).

Synthesis of a G1-Self-Immolative Dendrimer having Pyrenes Tail Units and a Photo-Labile Trigger Unit:

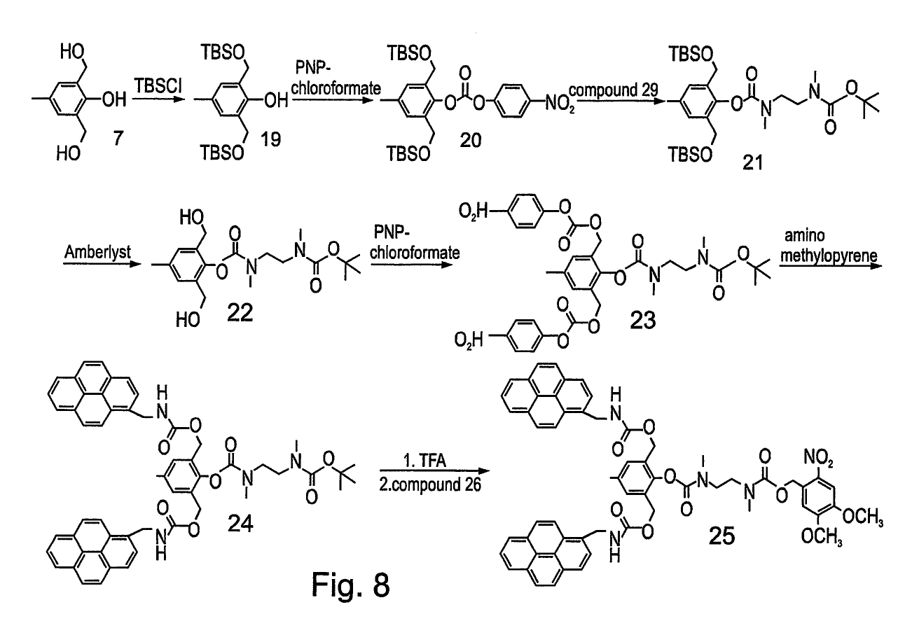

The total synthesis of a G1-self-immolative dendrimer having two pyrene molecules as tail units and a photo-labile trigger unit is presented in FIG. 8 and is detailed hereinbelow.

Synthesis of Compound 19 (FIG. 8): The commercially available 2,6-bishydroxymethyl-p-cresol, Compound 7 (2.0 grams, 11.9 mmol) was dissolved in 10 ml DMF and the solution was cooled to 0° C. Imidazole (1.6 grams, 23.8 mmol) and tert-butyldimethylsilyl chloride (TBSCl, 3.6 grams, 23.8 mmol) were added and the reaction mixture was allowed to warm to room temperature and was thereafter stirred for additional two hours. The reaction progress was monitored by TLC, using a mixture of 5:95 ethyl acetate (EtOAc):hexane as eluent. Once the reaction was completed, the reaction mixture was diluted with ether and was washed with water. The organic layer was dried over sodium sulfate, and the solvent was removed under reduced pressure. The crude product was purified by column chromatography on silica gel, using a mixture of 5:95 EtOAc:hexane as eluent, to give Compound 19 (4.0 grams, 84% yield) as a colorless oil.

¹H-NMR (200 MHz, CDCl₃): δ=8.03 (1H, s); 6.90 (2H, s); 4.82 (4H, s); 2.26 (3H, s); 0.94 (18H, s); 0.12 (12H, s).

Synthesis of Compound 20 (FIG. 8): Compound 19 (3.4 grams, 8.7 mmol) was dissolved in 100 ml dichloromethane. Triethylamine (Et₃N, 4.2 ml, 0.03 mol) and a catalytic amount of dimethylaminopyridine (DMAP) were added and the mixture was cooled to 0° C. A solution of 4-nitrophenyl (PNP) chloroformate (2.6 grams, 13 mmol) in 20 ml dichloromethane was added dropwise and the reaction mixture was stirred at room temperature for 20 minutes, while being monitored by TLC, using a mixture of 5:95 EtOAc:hexane as eluent. Once the reaction was completed, the mixture was diluted with dichloromethane and washed with HCl 1N. The organic layer was dried over sodium sulfate and the solvent was removed under reduced pressure. The crude product was purified by column chromatography on silica gel, using a mixture of 5:95 EtOAc:hexane as eluent, to give Compound 20 (43 grams, 87.8% yield) as a white powder.

¹H-NMR (200 MHz, CDCl₃): δ=8.31 (2H, d, J=9.0); 7.48 (2H, d, J=9.0); 7.22 (2H, s); 4.72 (4H, s); 2.38 (3H, s); 0.93 (18H, s); 0.09 (12H, s).

Synthesis of mono-BOC-N,N′-dimethylethylenediamine (Compound 29, FIG. 8): The commercially available N,N′-dimethylethylenediamine (10 grams, 113 mmol) was dissolved in 120 ml dichloromethane and the solution was cooled in ice water. A solution of di-t-butylcarbonate (8.3 grams, 38 mmol) in 60 ml of dichloromethane was added dropwise at 0° C. and the reaction mixture was allowed to warm to room temperature and was thereafter stirred overnight. The solvent was then removed under reduced pressure and the crude mixture was dissolved in EtOAc and was washed with brine. The organic solution was dried over magnesium sulfate and the solvent was removed under reduced pressure to yield Compound 29 (6.5 grams, 91% yield) as s pale yellowish oil.

¹H-NMR (200 MHz, CDCl₃): δ=3.3 (2H, t, J=6.6); 2.85 (3H, s); 2.70 (2H, t, J=6.6); 2.42 (3H, s); 1.43 (9H, s).

Synthesis of Compound 21 (FIG. 8): Compound 20 (5.4 grams, 9.7 mmol) was dissolved in 10 ml dimethylformamide (DMF) and Compound 29 (1.8 grams, 9.7 mmol) was added to the solution. The reaction mixture was stirred at room temperature for 20 minutes, while being monitored by TLC, using a mixture of 1:3 EtOAc:hexane as eluent. Once the reaction was completed, the solvent was removed under reduced pressure and the crude product was purified by column chromatography on silica gel, using a mixture of 1:3 EtOAc:hexane as eluent, to give Compound 21 (5.3 grams, 89% yield) as a white powder.

¹H-NMR (200 MHz, CDCl₃): δ=7.19 (2H, s); 4.62 (4H, s); 3.7-3.4 (4H, m); 3.14 (3H, s); 2.92 (3H, s); 2.35 (3H, s); 1.47 (9H, s); 0.92 (18H, s); 0.08 (12H, s).

Synthesis of Compound 22 (FIG. 8): Compound 21 (1.1 grams, 1.8 mmol) was dissolved in MeOH and Amberlyst 15 was added. The reaction mixture was stirred at room temperature for two hours, while being monitored by TLC, using a mixture of 1:3 EtOAc:hexane as eluent. Once the reaction was completed, the Amberlyst 15 was filtered out and the solvent was removed under reduced pressure. The crude product was purified by column chromatography on silica gel, using 100% EtOAc as eluent, to give Compound 22 (635 mg, 92% yield) as a white powder.

¹H-NMR (200 MHz, CDCl₃): δ=7.21 (2H, s); 4.53 (4H, s); 3.7-3.62 (2H, m); 3.52-3.47 (2H, m); 3.05 (3H, s); 2.95 (3H, s); 2.35 (3H, s); 1.49-1.44 (9H, m).

Synthesis of Compound 23 (FIG. 8): Compound 22 (343 mg, 0.9 mmol) was dissolved in EtOAc. PNP-chloroformate (543 mg, 2.7 mmol), Et₃N (0.4 ml, 2.7 mmol) and a catalytic amount of DMAP were added. The reaction mixture was stirred in room temperature for one hour, while being monitored by TLC, using a mixture of 1:1 EtOAc:hexane as eluent. Once the reaction was completed, the reaction mixture was diluted with methylene chloride and was washed with HCl 1 M and brine. The organic layer was dried over sodium sulfate. The solvent was removed under reduced pressure. The crude product was purified by column chromatography on silica gel, using a mixture of 3:7 EtOAc:hexane as eluent, to give Compound 23 (480 mg, 75% yield) as a white powder.

¹H-NMR (200 MHz, CDCl₃): δ=8.27 (4H, d, J=9.2); 7.38 (4H, d, J=9.2); 7.25 (2H, s); 5.26 (4H, s); 3.65-3.62 (4H, m); 3.20 (3H, s); 3.03 (3H, s); 2.39 (3H, s); 1.45 (9H, s).

Synthesis of Compound 24 (FIG. 8): Compound 23 (500 mg, 0.7 mmol) was dissolved in 5 ml DMF. The hydrochloride salt of aminomethylpyrene was added (394 mg, 1.5 mmol), followed by the addition of Et₃N (0.2 ml, 1.5 mmol). The reaction mixture was stirred at room temperature for 10 min, while being monitored by TLC, using a mixture of 1:1 EtOAc:hexane as eluent. The solvent was removed under reduced pressure and the product was purified by column chromatography on silica gel, using a mixture of 1:1 EtOAc:hexane as eluent, to give Compound 24 (575 mg, 92% yield) as a white powder demonstrating strong fluorescence when irradiated s by a standard UV lamp.

¹H-NMR (200 MHz, CDCl₃): δ=8.21-7.97 (18H, m); 7.15 (2H, s); 5.09-5.06 (8H, m); 3.25-3.27 (4H, m); 2.76 (3H, bs); 2.70 (3H, bs); 2.28 (3H, s); 1.37 (9H, s).

Synthesis of Compound 26: The commercially available 4,5-dimethoxy-2-nitrobenzyl-alcohol (150 mg, 0.7 mmol) and PNP-chloroformate (155 mg, 0.77 mmol) were dissolved in EtOAc. Et₃N (0.1 ml, 0.77 mmol) and catalytic amount of DMAP were added, and the reaction was stirred for 10 minutes, while being monitored by TLC, using a mixture of 1:3 EtOAc:hexane as eluent. Once the reaction was completed, the solvent was removed under reduced pressure and the product was purified by column chromatography on silica gel, using a mixture of 1:1 EtOAc:hexane as eluent, to give Compound 26 described hereinbelow (150 mg, 50% yield) as a pale yellow powder.

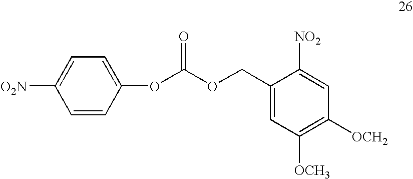

¹H-NMR (200 MHz, CDCl₃): δ=8.32 (2H, d, J=7.2); 7.78 (1H, s); 7.42 (2H, d, J=7.2); 7.11 (1H, s); 5.71 (2H, s); 4.03 (3H, s); 3.99 (3H, s).

Synthesis of Compound 25 (FIG. 8, G1-self-immolative dendrimer): Compound 24 (100 mg, 0.11 mmol) was reacted with 2 ml trifluoroacetic acid (TFA) to remove the BOC protecting group. The excess of the acid was removed under reduced pressure and the residue was dissolved in 2 ml DMF. Compound 26 (51.8 mg, 0.13 mmol) and 1 ml Et₃N were added and the solution was stirred for 10 minutes. DMF was then removed under reduced pressure and the crude product was purified by column chromatography on silica gel, using a mixture of 1:1 EtOAc:hexane as eluent, to give pure compound 25 as a pale yellow powder (86 mg, 75% yield).

HR-MS (MALDI): calculated for C₆₀H₅₃N₅O₁₂ 1058.3583 [M+Na]⁺, found 1058.3448.

Synthesis of a G2-Self-Immolative Dendrimer having Pyrenes as Tail Units and a Photo-Labile Trigger Unit:

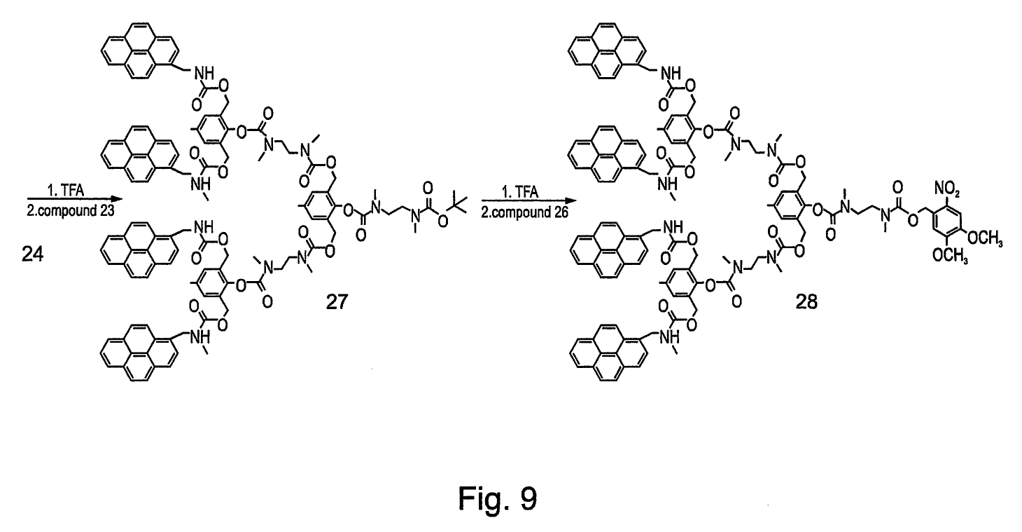

The total synthesis of a G2-self-immolative dendrimer having four pyrene molecules as tail units and a photo-labile trigger unit is presented in FIG. 9 and is detailed hereinbelow.

Synthesis of Compound 27 (FIG. 9): Compound 24 (250 mg, 0.28 mmol) was reacted with 2 ml TFA, to remove the BOC protecting group. The excess of the acid was removed under reduced pressure and the residue was dissolved in 2 ml DMF. Compound 23, prepared as described hereinabove, (95 mg, 0.13 mmol) and 1 ml Et₃N were added thereto and the solution was stirred for 10 minutes. Once the reaction was completed, the DMF was removed under reduced pressure and the crude product was purified by column chromatography on silica gel, using a mixture of 1:1 EtOAc:hexane as eluent, to give pure compound 27 (125 mg, 50% yield) as a pale yellow powder having a typical ¹H-NMR spectrum.

Synthesis of Compound 28 (FIG. 9, G2-self-immolative dendrimer): Compound 27 (105 mg, 0.05 mmol) was reacted with 2 ml TFA, to remove the BOC protecting group. The excess of the acid was removed under reduced pressure and the residue was dissolved in 2 ml DMF. Compound 26 described hereinabove (23 mg, 0.06 mmol) and 1 ml Et₃N were added and the solution was stirred for 10 minutes. DMF was thereafter removed under reduced pressure and the crude product was purified by column chromatography on silica gel, using a mixture of 1:1 EtOAc:hexane as eluent, to give pure compound 28 as a pale yellow powder (44.5 mg, 41% yield).

HR-MS (MALDI): calculated for C₁₂₆H₁₁₅N₁₁O₂₄ 2188.8014 [M+Na]⁺, found 2188.8336.

### Example 5

### Analysis of the Release of Pyrene Molecules from G1- and G2-Self-Immolative Dendrimers

General protocol: The self-immolative dendrimer (SID) (2 mM) was dissolved in 4 ml DMSO to yield a 500 μM stock solution. The latter was further diluted by a MeOH:dichloromethane mixture (1:1), to yield 50-μM solutions, which were used directly for monitoring the release reaction. All solutions were kept at 37° C. prior to use. The release of the tail units was monitored by an HPLC assay, using C-18 column, wavelength—360 nm, eluent—acetonitrile:water; programmed gradient, flow rate—1 ml/min.

Kinetic Calculations:

The self-immolative mechanism for releasing tail units from the dendrimers of the present invention was demonstrated by kinetic measurements. The kinetic experiments and calculations conducted were based on the assumption that the self-immolative fragmentation of the G1 amine-intermediate Compound 2 (FIG. 3) is performed according to a first order reaction and therefore, a plot of the natural logarithm of [2] as a function of time should present a good correlation with a linear equation (y=ax+b), as is derived from the calculations presented in equations 1-4 below:
d[2]/dt=−k₁[2] (1)
d[2]/[2]=−k₁dt (2)
ln[2]=ln[2]₀−k₁t (3)
[2][2]₀e\hat{ }−k₁t (4)

The kinetic measurements were performed with the G1- and G2-SIDs prepared in Example 4 (having a photo-labile trigger unit and pyrene tail units), according to the general protocol described hereinabove. The solutions were irradiated with UV light (λ=360 nm), so as to cleave the photo-labile trigger unit and 10% Et₃N was added, so as to initiate the self-immolative reactions (a mild basic media is needed for the quinone-methide rearrangement). The reaction progress was monitored by HPLC, as is described hereinabove.

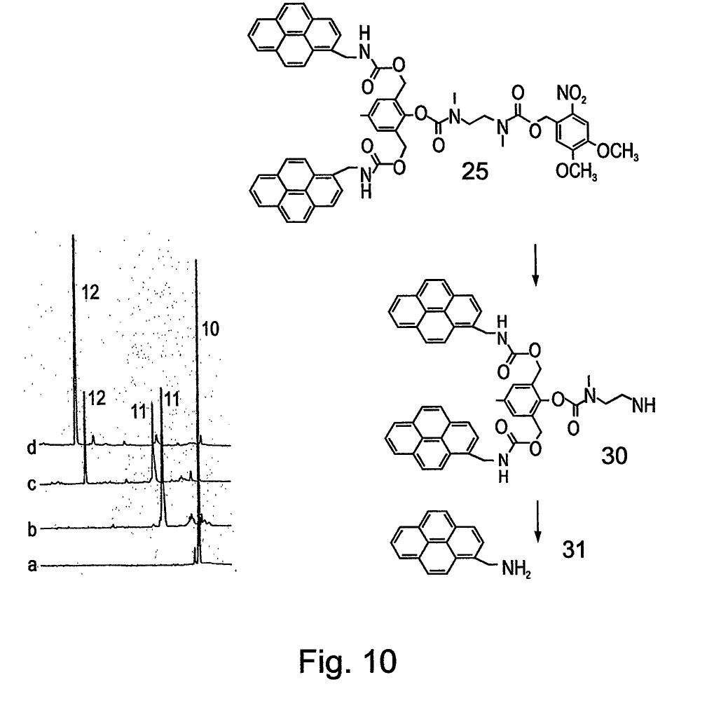

FIG. 10 presents the HPLC chromatograms obtained before irradiation and 0, 4 and 11 hours after irradiation of a solution of the G1-SID Compound 25. As is shown in FIG. 10, the cleavage of the photo-labile trigger unit of Compound 25 generated the amine-intermediate Compound 30, which gradually degraded to the aminomethylpyrene tail units 31 through the previously described self-immolative process (see, FIG. 3). The release of the aminomethylpyrene molecules was completed after 11 hours.

Since no intermediates other than Compound 30 were observed, as is shown in FIG. 10, it was concluded that the rate limiting step of the self-immolative sequence is the cyclization of Compound 30, to form an N,N′-dimethylurea derivative and a phenol (see, model Compound 3, FIG. 3), which is rapidly rearranged to release the tail units. The amine-intermediate Compound 30 was characterized by HRMS-analysis and by HPLC comparison to a reference compound.

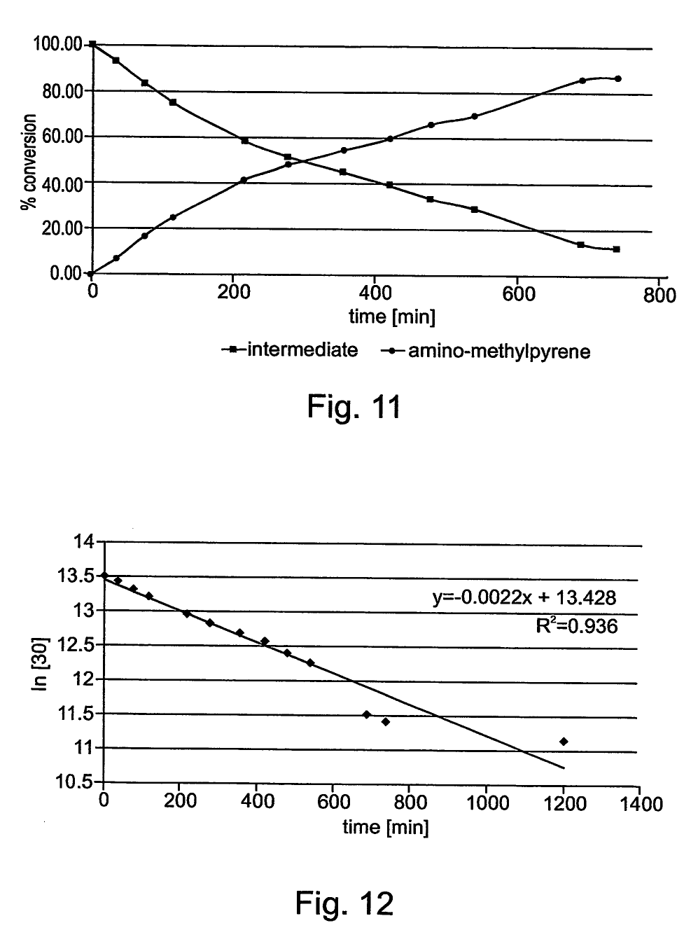

FIGS. 11 and 12 present the fragmentation (in %) of intermediate 30 to release the free tail units (aminomethylpyrene, 31), and the natural logarithm of the concentration of Compound 30, as a function of time, respectively.

The first order rate constant, k₁, was calculated from the slope of the linear fit (FIG. 12), and was found to be 2.2e-3 min⁻¹.

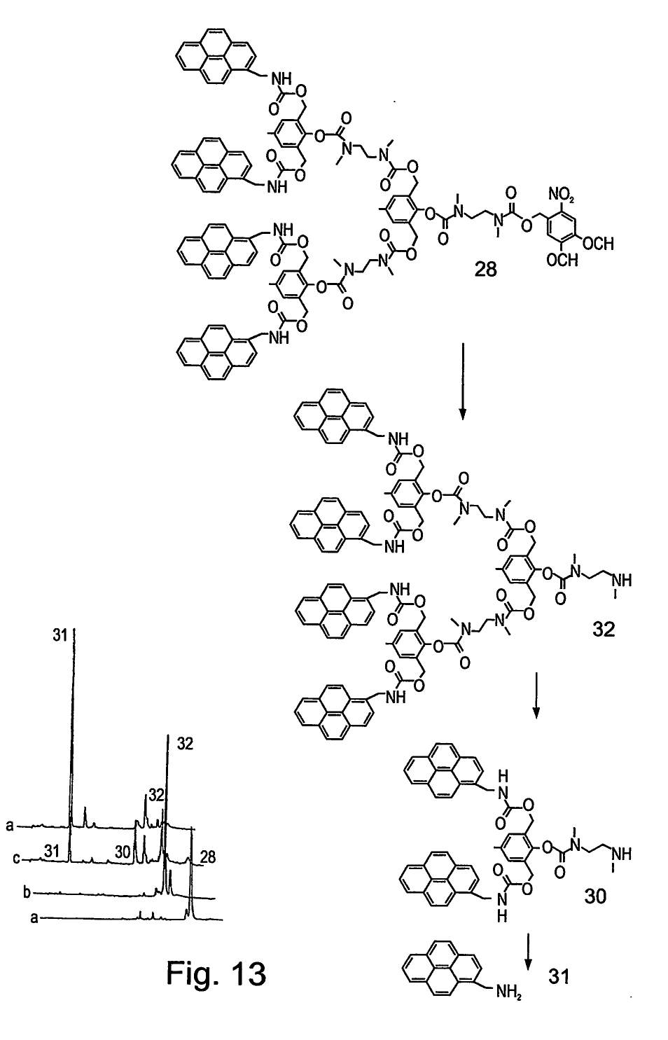

A similar analysis was performed with the G2-SID Compound 28. FIG. 13 presents the HPLC chromatograms obtained before irradiation and 0, 6 and 20 hours after irradiation of a solution of the G2-SID Compound 28. As is shown in FIG. 13, the cleavage of the photo-labile trigger unit of Compound 25 generated the amine-intermediate Compound 32, which gradually degraded to the amine intermediate Compound 30 and thereafter self-immolatively released the aminomethylpyrene tail units 31. The release of the aminomethylpyrene molecules was completed after 21 hours.

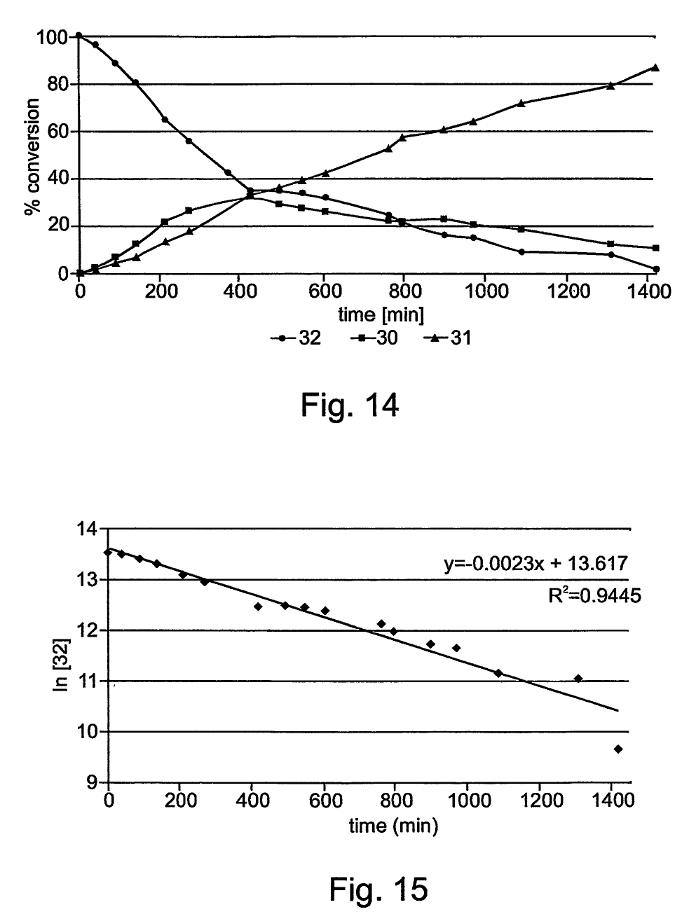

FIG. 14 presents the conversion of intermediate 32 to intermediate 30, and the latter self-elimination to release the aminomethylpyrene molecules 31.

FIG. 15 presents the natural logarithm of the concentration of intermediate Compound 32 as a function of time.

As is shown in FIGS. 14 and 15, the self-immolative mechanism of the G2 dendrimer shows a similar kinetic pattern as compared with the G1 dendrimer. The conversion of intermediate Compound 32 can therefore be regarded as a first order reaction, and consequently, the rate constant k₂ can be calculated in a-similar manner to that of k₁. Hence, k₂ was calculated from the slope of the linear fit (FIG. 15), and was found to be identical to k₁.

The identical values accepted for k₁ and k₂ are attributed to the identical rate-determining step, namely the cyclization of the amine-intermediate, of both the G2-dendrimer and G1-dendrimer fragmentation reactions. Thus, throughout the self-immolation of the G2-dendrimer, intermediate 30 is formed and fragmentized at the same time.

However, in order to support the experimental findings and the theoretical kinetics concluded therefrom, mathematical calculations have been conducted in accordance with the theory described above and the theoretical expected results have been compared with the experimental data.

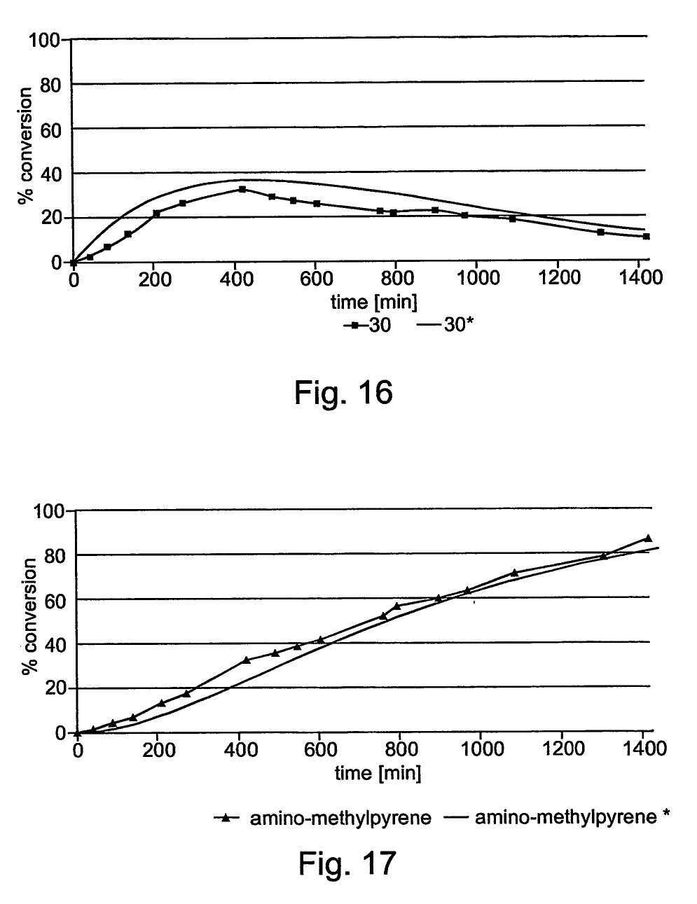

The theoretical calculations were based on a reaction model consisted of two steps:
Step 1: 32→2×30
Step 2: 30→2×31
   Hence, the reaction kinetics of step 1 was calculated from equations 5-8 below, which are based on equations 1-4 (presented and discussed hereinabove):
   d[32]/dt=−k₂[32] (5)
   d[32]/[32]=−k₂dt (6)
   ln[32]=ln[32]₀−k₂t (7)
   [32]=[32]₀e\hat{ }−k₂t (8)
   whereas the reaction kinetics of step 2 was calculated from equations 9-12 below:
   d[30]/dt=−k₁[30] (9)
   d[30]/[30]=−k₁dt (10)
   ln[30]=ln[30]₀−k₁t (11)
   [30]=[30]₀e\hat{ }−k₁t (12)
   Accordingly, equations 13 and 14 below lead to a mathematical function that describes the concentration of intermediate 30 as function of time. The solution of equation 14 is based on the findings that k₁ equals k₂. Equation 15 provides a comparison between the experimental and the calculated results, which is further demonstrated in FIG. 16.
   d[11]/dt=2k₂[14]−k₁[11] (13)
   d[11]/dt+k₁[11]=2k₂[14]₀e\hat{ }−k₂t (14)
   [11]=2k[14]₀te\hat{ }−kt (15)

As is shown in FIG. 16, a good correlation between the theoretical and experimental calculations, regarding curve shape and tₘₐₓ value, has been obtained.

The expression calculated in equation 15 for the concentration of intermediate Compound 30, has been submitted into the second step rate expression presented in equation 16 below, to yield equation 17. The solution of this equation is a mathematical description of the formation of the free tail units 31 as a function of time for a G2-SID fragmentation, as presented in equation 18 below:
d[31]/dt=2k[30] (16)
d[31]/dt=4k²[32]₀te\hat{ }−kt (17)
[31]=4[32]₀(1−(e\hat{ }−kt)−(kte\hat{ }−kt)) (18)

As is shown in FIG. 17, an excellent correlation was found between the calculated formation of 31 as a function of time and the measured experimental results obtained for the release of the aminomethylpyrene free molecules.

The data presented in FIGS. 16 and 17 provide substantial support for the self-immolative mechanism and the kinetic characterization of the SID system of the present invention. Furthermore, these data shows that the kinetic analysis described hereinabove for the G1- and G2-SIDs is a reliable method for characterizing SID systems in general and further provides for a better understanding and evaluation of the kinetic contributions of introducing different substituents to the core ring, and of modifying the spacer component of the SID system of the present invention.

### Example 6

### Synthesis of a G3-Self Immolative Dendrimer

Attempts to synthesize a G3-SID having eight aminomethylpyrene molecules as the tail units have not succeeded so far, apparently due to steric hindrances. Hence, it was hypothesized that a G3-SID having a structure that is based on the G1- and G2-SIDs, Compounds 25 and 28, described hereinabove in Example 4, and a smaller molecule as the tail units would be synthesizable. It was further hypothesized that 4-nitroaniline, which is a significantly smaller molecule than pyrene, and is further easy to detect in its free form, due to its yellow color, could serve as an alternative tail group in synthesizing and characterizing a G3-SID according to the present invention. Indeed, a G3-SID having eight 4-nitroaniline molecules as its tail units and a BOC trigger unit, Compound 35, has been successfully synthesized, thus confirming the described hypothesis.

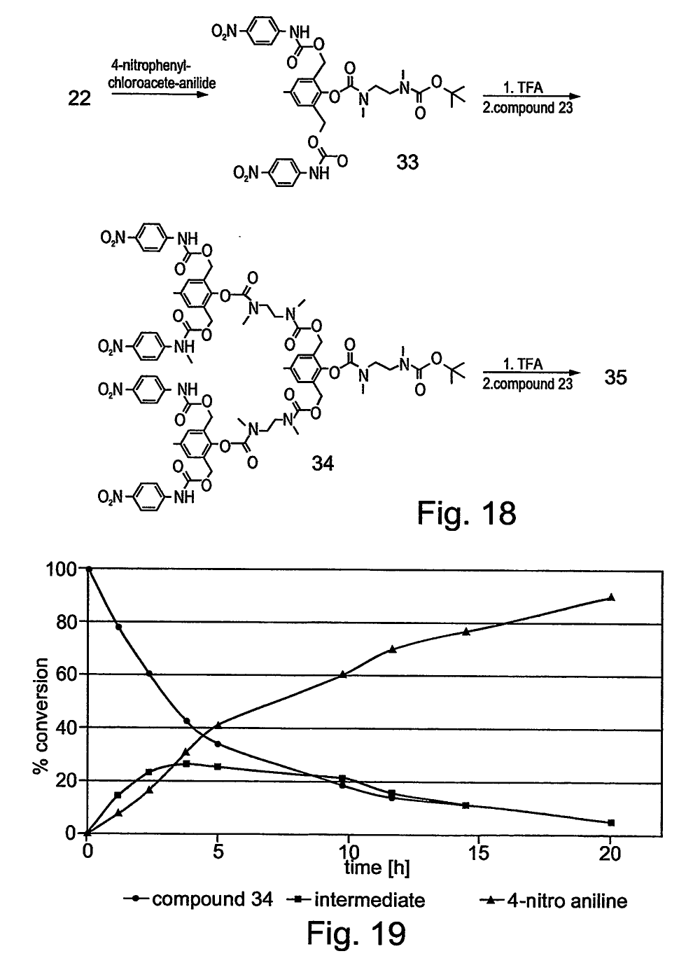

The synthesis of the G3-SID Compound 35, as well as of its corresponding G1- and G2-SIDs, Compounds 33 and 34, respectively, is presented in FIG. 18 and is detailed hereinbelow:

Synthesis of Compound 33 (FIG. 18, a G1-SID): Triphosgen (110 mg, 0.37 mmol) was dissolved in 10 ml EtOAc. The mixture was cooled to 0° C. and a solution of 4-nitroaniline (155 mg, 1.12 mmol) and Et₃N (113 mg, 1.12 mmol) in 5 ml EtOAc was added dropwise. The mixture was stirred for 10 minutes and thereafter Compound 22 (107 mg, 0.28 mmol, see, FIG. 8) and DMAP (68.32 mg, 0.56 mmol) were added. The reaction mixture was stirred for 1.5 hours, while being monitored by TLC, using 100% EtOAc as eluent. Et₃N was then added and the reaction mixture was stirred overnight. The salts were thereafter filtered and the solution was washed with HCl 1N. The organic layer was dried over magnesium sulfate and the solvent was removed under reduced pressure. The crude product was purified by flash chromatography as described hereinabove, using a mixture of 2:3 EtOAc:hexane as eluent, to give the pure Compound 33 as a yellow powder (190 mg, 96% yield).

¹H-NMR (200 MHz, CDCl₃): δ=8;16 (4H, d, J=8); 7.64 (4H, d, J=8); 5.30 (4H, s); 3.50-2.91 (10H, m); 2.36 (3H, s); 1.47 (3H, d, J=4).

Synthesis of Compound 34 (FIG. 18, a G2-SID): Compound 33 (200 mg, 0.282 mmol) was reacted with 2 ml TFA, to thereby remove the BOC protecting group. The excess of the acid was removed under reduced pressure and the residue was dissolved in 2 ml DMF. Compound 23 (89 mg, 0.128 mmol), followed by 1 ml triethylamine, were added thereto and the reaction mixture was stirred for 1.5 hours, while being monitored by TLC, using a mixture of 3:1 EtOAc:hexane as eluent. DMF was then removed under reduced pressure and the crude product was purified by flash chromatography, using a mixture of 65:35 EtOAc:hexane as eluent, followed by preparative TLC, using a mixture of 96:4 dichloromethane:methanol as eluent, to give pare Compound 34 as a white powder (163 mg, 77% yield)

HR-MS (MALDI): calculated for C₇₇H₈₆N₁₄O₂₈ 1678.5735 [M+Na]⁺, found 1678.6264.

Synthesis of Compound 35 (FIG. 18, a G3-SID): Compound 34 (130 mg, 0.078 mmol) was reacted with trifluoroacetic acid, to thereby remove the BOC protecting group. The obtained amine salt was then dissolved in anhydrous DMF and Compound 23 (23 mg, 0.0315 mmol) and triethylamine were added thereto. The reaction mixture was stirred for 3 hours and the solvent was thereafter removed under reduced pressure. The crude product was purified by preparative TLC, using a mixture of 96:4 dichloromethane:methanol, yielding pure Compound 35 as a white powder (55 mg, 50% yield), characterized by a typical ¹H-NMR spectrum.

In control experiments that were conducted, both G2- and G3-SIDs, Compounds 34 and 35, were found to be highly stable as long as the trigger unit was not removed, as no decomposition of these compounds was observed for at least 72 hours.

The release of eight 4-nitroaniline tail units from Compound 35 was confirmed by HPLC analysis, as is described in detail hereinafter in Example 7.

### Example 7

### Analysis of the Release of 4-Nitroaniline Molecules from G2- and G3-Self-Immolative Dendrimers

Release of 4-nitro-aniline from the G2-SID Compound 34: The activation of the 4-nitroaniline G2 dendrimer Compound 34 was performed by chemically removing the BOC trigger group of compound 34 with trifluoroacetic acid. The obtained corresponding amine salt was used for the preparation of stock solutions in DMSO:Chremophor (1:1). A 100 μM solution of compound 34 in MeOH:dichloromethane, 1:1, was activated with 10% triethylamine and the release of 4-nitroaniline was monitored by an HPLC assay, using a C-18 analytical column, wavelength—348 nm, a gradient eluent of acetonitrille:water—0-20 minutes: 30%-100% acetonitrile, 20-25 minutes 100% acetonitrile, 25-30 minutes 100%-30% acetonitrile, flow rate—1 ml/min.

FIG. 19 presents the fragmentation of the amine salt of Compound 34 into four 4-nitroaniline molecules via an amine intermediate, as a function of time, based on the data obtained from the HPLC analysis.

Release of 4-nitro-aniline front the G3-SID Compound 35: The activation of the G3-SID Compound 35 was performed by removing the BOC trigger group with TFA. The thus formed amine salt was used for the preparation of stock solutions in DMSO:Chremophor (1:1). A 50 μM solution of compound 34 in MeOH:dichloromethane, 1:1, was activated with 10% triethylamine and the release of 4-nitroaniline was monitored by an HPLC assay, as is described hereinabove.

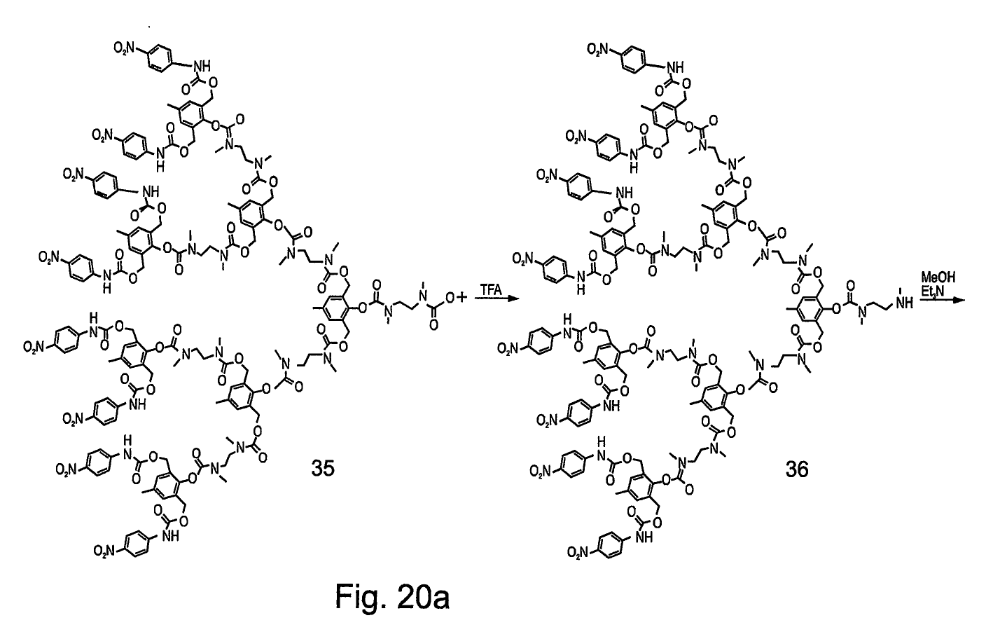

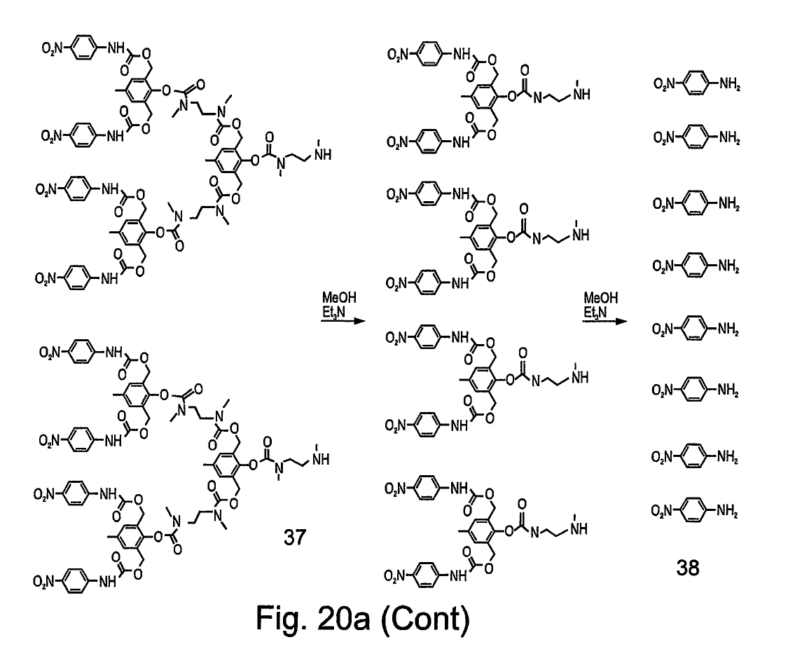

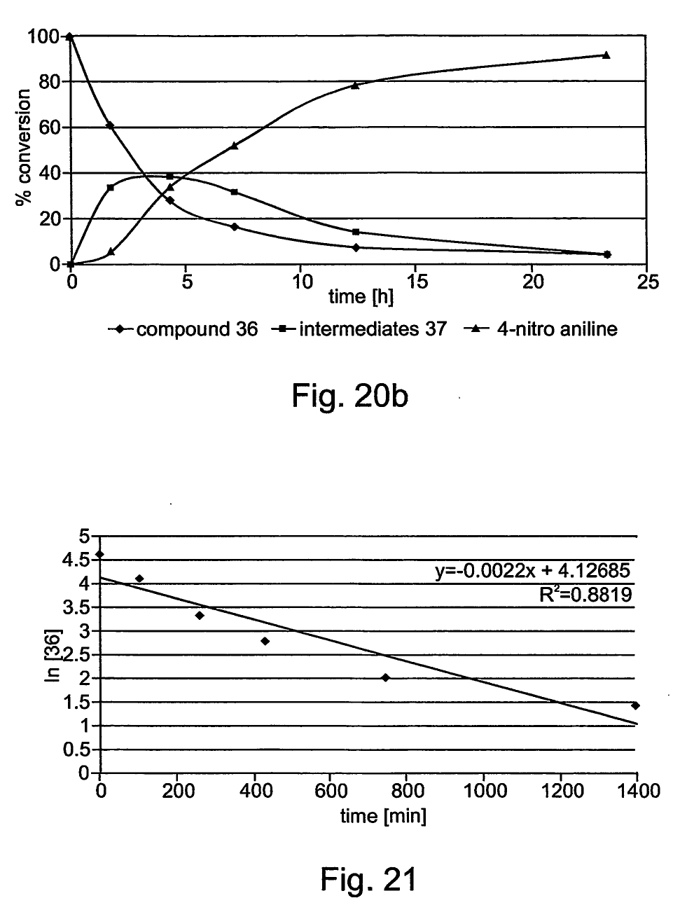

The expected pattern of the self-immolative process is described in FIG. 20a: The amine intermediate Compound 36, obtained by removing the trigger group, degrades into the amine intermediate Compound 37, which thereafter degrades into the amine intermediate Compound 38, which is degraded to release eight 4-nitroaniline molecules.

FIG. 20b presents the data obtained by the HPLC assay, which clearly demonstrate the release of the tail units from Compound 35 via a self-immolation mechanism. As is shown in FIG. 20b, the amine intermediates Compound 37 and 38 were gradually generated and disappeared to finally release eight molecules of 4-nitroaniline.

As is further demonstrated in FIG. 20b, the self-immolative mechanism of the G3 dendrimer shows a similar kinetic pattern as compared with the G1- and G2-dendrimers analysed in Example 5 hereinabove. Hence, as is presented in FIG. 21, the conversion of the amine intermediate Compound 36 was considered to be a first order reaction, and consequently, the rate constant k₃ was calculated in a similar manner to that of k₁ and k₂. As is further indicated in FIG. 21, k₃ was found to be identical to k₁ and k₂ (see, FIGS. 12 and 15).

### Example 8

### Design and Synthesis of Self-Immolative Dendrimers having Drugs as Tail Units and an Enzymatic Trigger Unit

The self-immolative dendrimer model designed hereinabove can be further used as a multi-prodrug by incorporating drug molecules as its tail units and an enzymatic substrate as the trigger unit, such that a multi-number of drug molecules are released upon a single enzymatic cleavage.

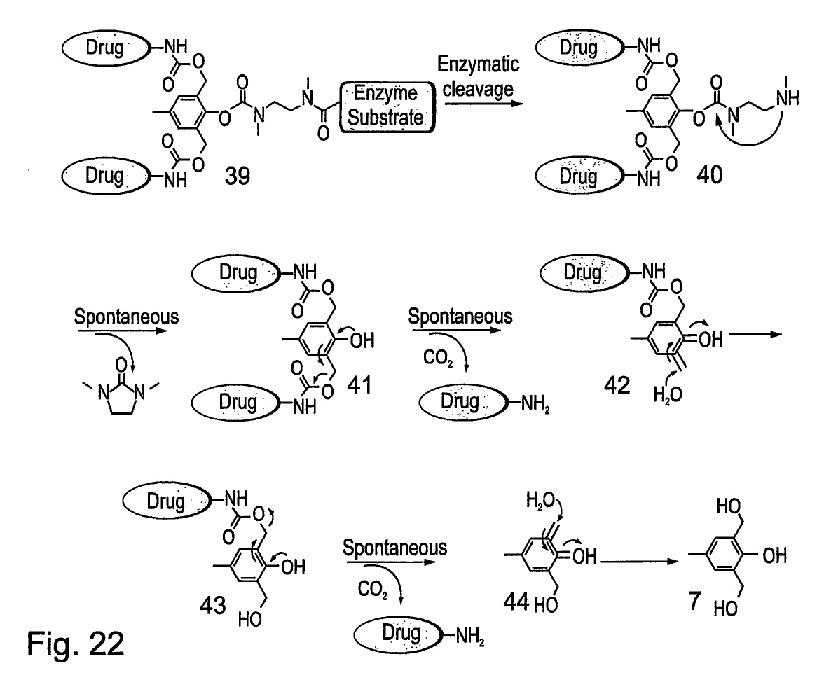

A representative example of such a SID model is presented in FIG. 22. This model is based on the model described hereinabove, in Example 1 (see, FIG. 3), and includes the commercially available 2,6-bishydroxymethyl-p-cresol, Compound 7, as the basic unit. A multi-prodrug G1-SID, according to this model, Compound 39, includes two drug molecules that are attached through a carbamate linkage to the two hydroxybenzyls of the basic unit and an enzymatic trigger unit that is attached to the phenol functionality via a short N,N′-dimethylethylenediamine spacer. Upon an enzymatic cleavage, the self-immolative reactions sequence is initiated, to form the amine intermediate Compound 40, which undergoes a spontaneous cyclization to form an N,N′-dimethylurea derivative and a phenolic Compound 41. The generated phenol 41 undergoes double 1,4-quinone-methide rearrangements (Compounds 42, 43 and 44), followed by spontaneous decarboxylations, to thereby liberate the drug molecules.

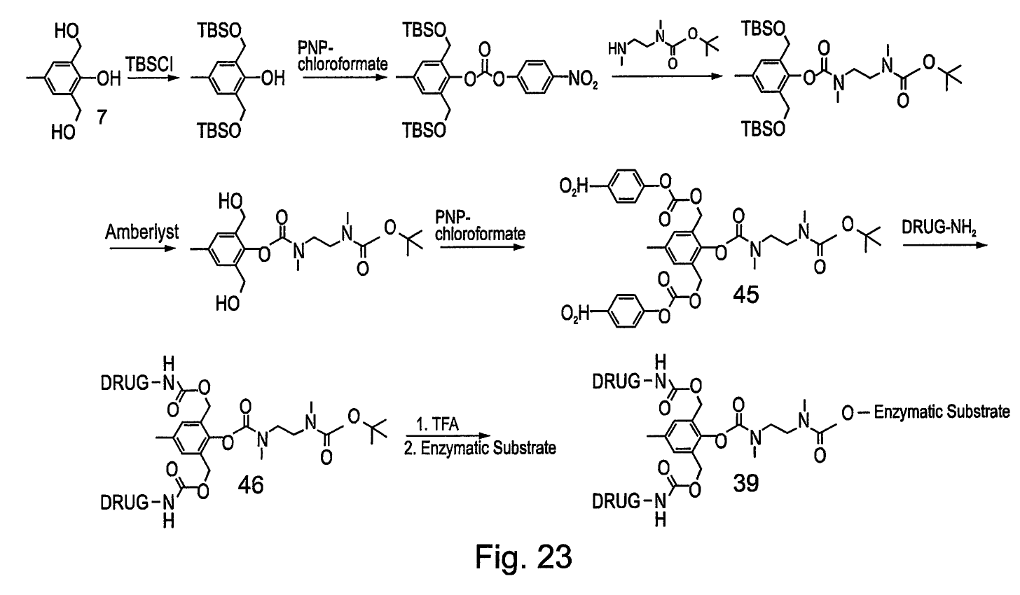

The general synthesis of the multi-prodrug G1-SID, Compound 39, is described in FIG. 23. The dicarbonate Compound 45 is synthesized according to the general synthesis described hereinabove (see, Example 1 and FIG. 4), by protecting the hydroxybenzyl groups, reacting the phenol functionality with p-nitrophenyl-chloroformate and thereafter with the short spacer N,N′-dimethylethylenediamine having a BOC-protecting group at its end, deprotecting the hydroxybenzyl groups and thereafter reacting the resulting diol with p-nitrophenyl-chloroformate. Compound 45 is then reacted with two equivalents of drug units having a free amine group, to give Compound 46. The latter is reacted with TFA, to remove the BOC protecting group and thereby generate an amine-salt, which is reacted in situ with an enzymatic substrate, to afford the G1-SID prodrug Compound 39.

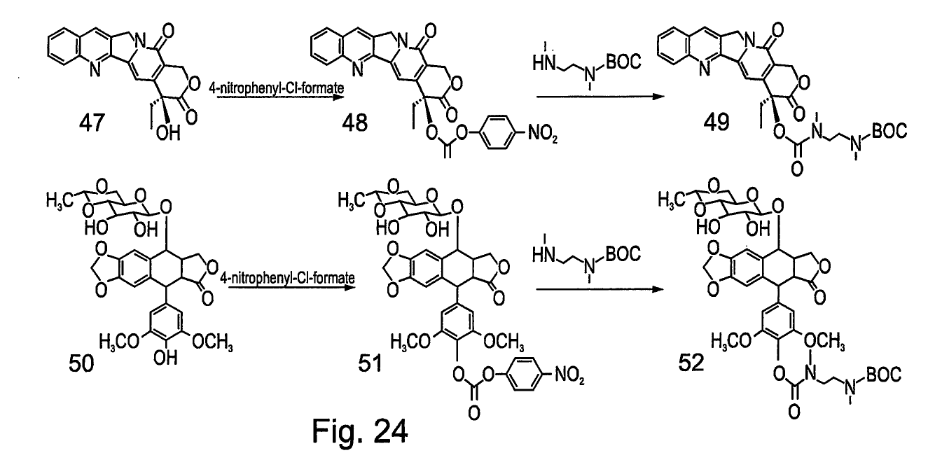

The general synthesis described hereinabove can be used to directly incorporate in the SIDs of the present invention drugs that have a free amine group, by attaching the drug molecules to the basic unit via a carbamate linkage. A representative example of such a drug is the anti-cancer drug doxorubicin. However, as some drugs do not have the required free amine functionality, another synthetic route has been developed in order to incorporate such drugs in the SIDs of the present invention. This synthetic route is exemplified in FIG. 24 with the hydroxy anti-cancer drugs camptothecin 47 [12] and etoposide 50 [13], and includes coupling the self-immolative spacer N,N-dimethyletylenediamine to the hydroxyl functionality of the drugs, via a carbamate linkage. The incorporation of such an amine spacer masks the hydroxy group of the drug and exchange it to an amine functionality that can be attached to the basic unit, while being spontaneously removed to unmask the hydroxy-drug group through spontaneous self-cyclization to form N,N-dimethylurea derivative, as is shown, for example, in FIGS. 3 and 22.

As is described in FIG. 24, the coupling of the diamine spacer to camptothecin 47 and etoposide 50 was performed by reacting the drug with p-nitrophenyl-chloroformate, to give the corresponding carbonates Compounds 48 and 51, respectively, which were further reacted with mono-BOC-N,N-dimethyletylene-diamine, to afford the BOC-protected amine spacer-drug conjugates 49 and 52, respectively. The incorporation of these conjugates into the SIDs of the present invention is performed by removing the BOC protecting group by reacting Compounds 49 and 52 with TFA, and reacting the resulting amine salts of the conjugates with the dicarbonate Compound 45 (see, FIG. 22).

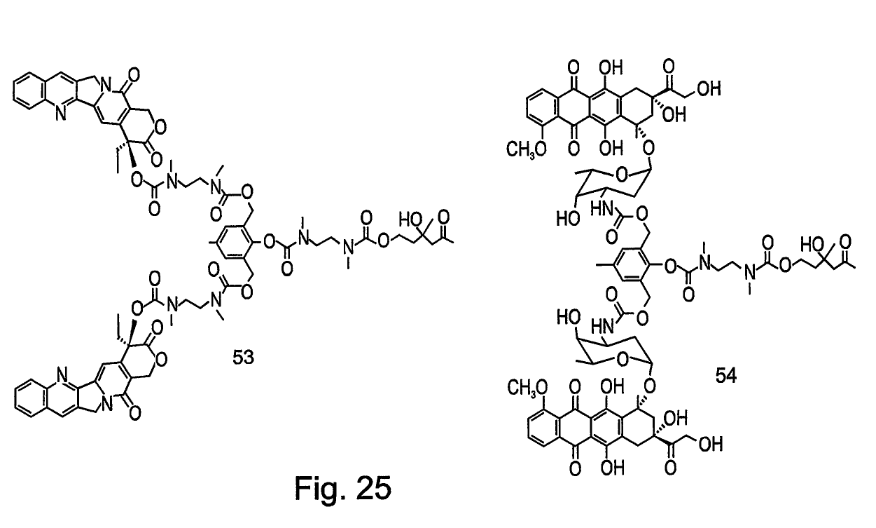

According to the synthetic pathways described hereinabove, representative examples of the multi-prodrug G1-SIDs of the present invention have been synthesized. FIG. 25 presents the chemical structures of a G1-SID that has two doxorubicin molecules as its tail units (Compound 53) and a G1-SID that has two camptothecin molecules as its tail units (Compound 54). The doxorubicin molecules are attached directly to the basic unit whereas the camptothecin molecules are attached to the basic unit through a self-immolative spacer. Both SIDs have a retro-aldol retro-Michael enzymatic trigger unit, which is known to be cleaved by the catalytic antibody 38C2 [14-16].

### Example 9

### Activity Assays of Self-Immolative Dendrimers having Drugs as Tail Units and an Enzymatic Trigger Unit

The G1-SID Compound 53, having two doxorubicin units as the tail units and a retro-aldol retro-Michael substrate of antibody 38C2 as the trigger unit, prepared as described in Example 8, was chosen as the first module for testing the therapeutic activity of the multi-prodrug SI)s of the present invention.

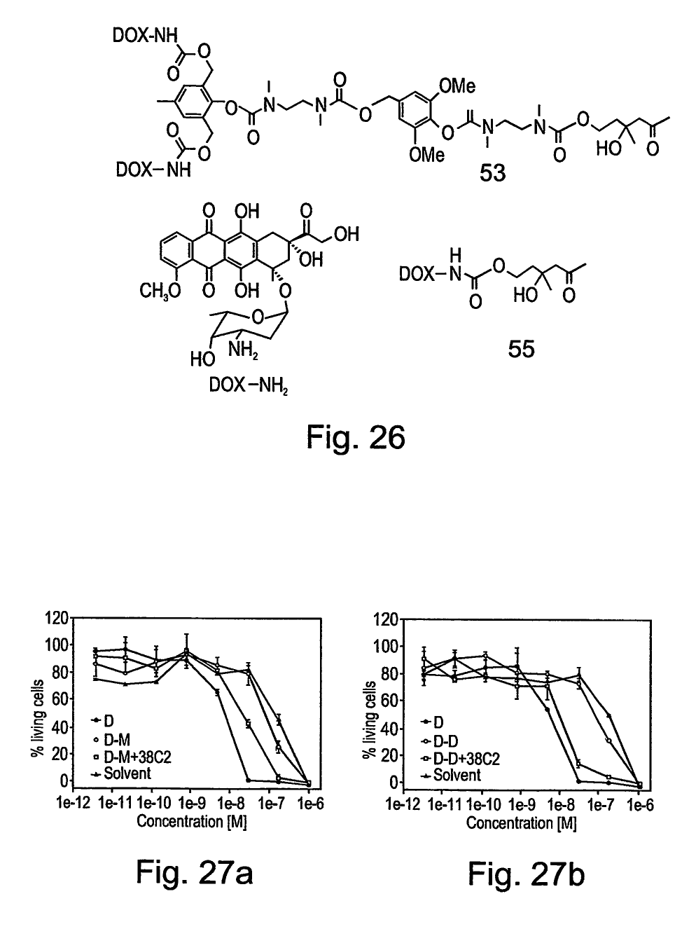

Hence, the cell-growth inhibition activity of Compound 53 was tested and compared with the inhibition activity of free doxorubicin and of a previously reported prodrug of doxorubicin 55, in which one molecule of drug is directly attached to a retro-aldol retro-Michael substrate of antibody 38C2 [15-17]. The structure of the three tested compounds is presented in FIG. 26.

The activity of the compounds was tested in vitro, using a cell-growth inhibition assay of Molt3 cell line, as is described, for example, by Shabat et al. [15-17]. Due to low solubility of the SID Compound 53 in the reaction medium, Cremophor EL was used as a co-solvent agent. The results obtained from comparative assays conducted with the monoprodrug 55 and free doxorubicin and with the SID prodrug 53 and free doxorubicin are presented in FIGS. 27a and 27b, respectively.

As is demonstrated in FIGS. 27a and 27b, the solvent control experiments show that most of the compound toxicity of both, the SID dimeric prodrug 53 (D-D, FIG. 27b) and the mono prodrug 55 (D-M, FIG. 27a) is derived from the co-solvent Cremephor EL. As is further demonstrated in FIG. 27b, the IC50 values obtained for both prodrugs 53 and 55 in the presence of the toxic co-solvent are about 50-folds higher than the IC50 value of the free doxorubicin (denoted as D). However, it is further shown in FIGS. 27a-b that the addition of the catalytic antibody 38C2 resulted in enhanced activity of the SID prodrug 53, as compared with its effect on the activity of the monoprodrug 55. The results presented in FIGS. 27a-b further show that the toxicity of both prodrugs remained almost identical throughout the assay, indicating the relative stability of the drugs linkages within the SID of the present invention.

As the incorporation of doxorubicin into the SID prodrug model of the present invention were found to be associated with solubility problems, additional tests were performed with the G1-SID prodrug Compound 54, which has camptothecin (CPT) molecules as its tail units (as is described in Example 8 and FIG. 25).

First, the enzyme-activated self-immolative process of releasing free CPT was verified by incubating the camptothecin prodrug 54 with catalytic antibody 38C2 and monitoring the appearance of free camptothecin, using a reverse-phase HPLC assay. As was expected, a signal of camptothecin was gradually appearing in the HPLC chromatogram.

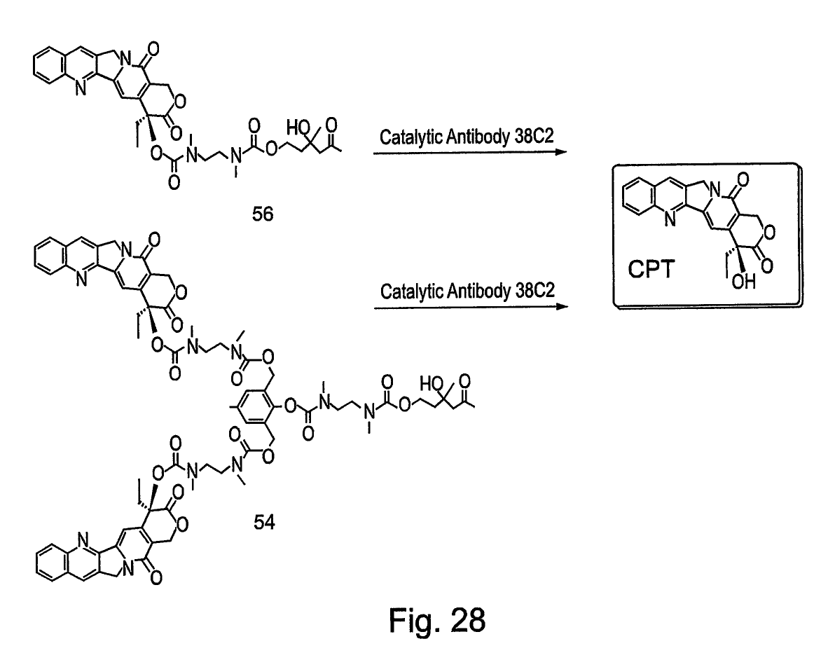

The release of free CPT from the G1-SID 54 was thereafter compared to that of the known monoprodrug of CPT, 56, in which one molecule of drug is attached to a retro-aldol retro-Michael substrate of antibody 38C2 via an N,N-dimethyletylene-diamine spacer [15-17], as is shown in FIG. 28. Both prodrugs were incubated with catalytic antibody 38C2 and the release of free CPT was monitored by HPLC. As was expected, the results indicated a similar disappearance rate of both prodrugs 54 and 56 and an appearance of a double-sized signal of free CPT in the SID prodrug 54, as compared with the monoprodrug 56 assay.

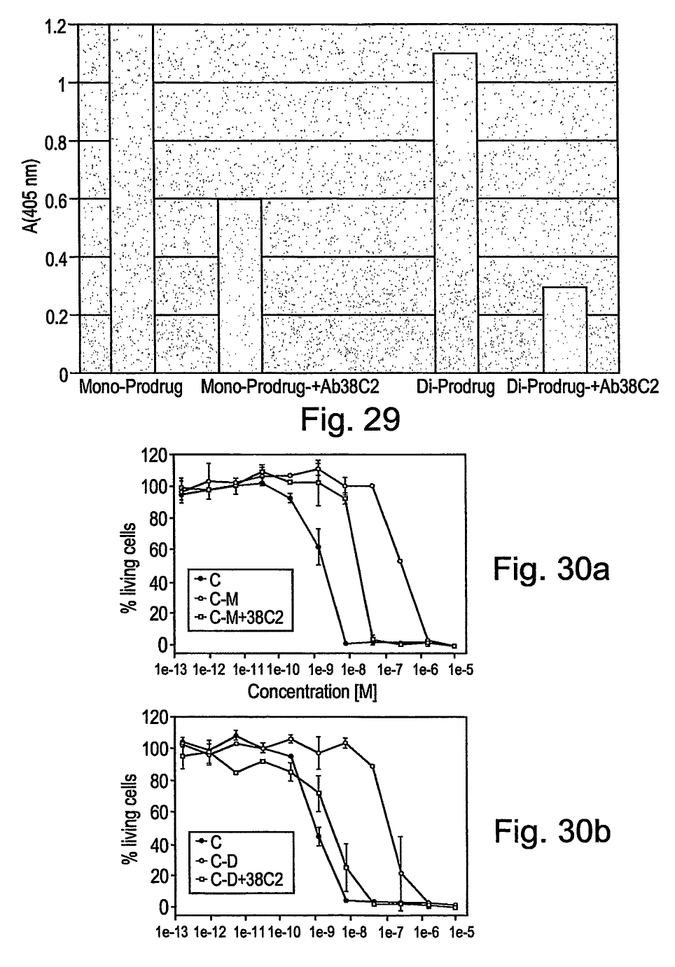

The anti-proliferative effect of the SID prodrug 54 and the mono prodrug 56 was evaluated by quantifying human colon carcinoma cell line LIM1215 growth in the presence of a range of concentrations of the prodrugs, with and without the catalytic antibody 38C2. The cells were lysed 120 hours after drug addition and the activity of the cytoplasmic enzyme lactate dehydrogenase released from the cells was assayed using a color reaction. Representative results are presented in FIG. 29.

As is shown in FIG. 29, at a concentration of 2.5 μM, both prodrugs exerted almost no toxicity, whereby in the presence of the catalytic antibody 38C2 strong cell growth inhibition was observed. However, FIG. 29 clearly demonstrates the twice-higher toxicity of the SID prodrug 54 as compared with the monoprodrug 56, which aligns with the HPLC results delineated hereinabove.

The cell-growth inhibition activity of Compound 54 was further tested and compared with the inhibition activity of free CPT and of the monoprodrug 56, using the cell-growth inhibition assay of Molt3 cell line described hereinabove. The results obtained from comparative assays conducted with the monoprodrug 56 and free CPT and with the SID prodrug 54 and free CPT are presented in FIGS. 30a and 30b, respectively.

As is shown in FIGS. 30a and 30b, the IC50 values obtained for prodrugs 56 (C-M, FIG. 30a) and 54 (C-D, FIG. 30b) were found to be almost identical, both about 200-folds higher than the IC50 value of free CPT (denoted as C). As is further shown in FIGS. 30a and 30b, addition of the catalytic antibody 38C2 resulted in enhanced inhibition activity of both prodrugs. However, as is clearly demonstrated in FIGS. 30a and 30b, again the effect of 38C2 on the toxicity of the SID prodrug 54 was found to be much higher as compared with its effect on the mono prodrug 56.

These results clearly demonstrate the advantages of the SID prodrugs of the present invention over the presently known prodrugs, by showing that identical concentrations of enzymatic protein release double amounts of active anticancer drug when G1-self-immolative dendrimer prodrug is applied. These results further implicate that this effect can be increased using higher generations of the SIDs of the present invention. As selective chemotherapy is highly depended on the ability to generate high local concentration of active drug at the tumor site, such enhanced local release of the drug is highly beneficial.

### Example 10

### Self-Immolative Dendrimers as Sensors

The SIDs of the present invention can be further used as sensors of, for example, enzymatic activity. Such a sensor SID can be obtained, for example, by incorporating a specific enzymatic substrate as the trigger unit of the self-immolative dendrimer and fluorogenic molecules that generate new chromophores upon liberation from the dendrimer as the tail units.

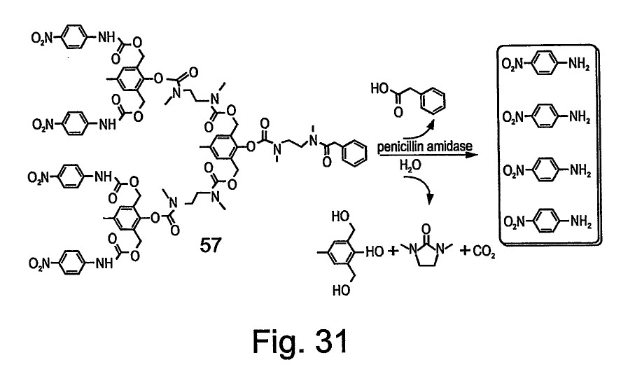

FIG. 31 presents a representative example of an enzymatic sensor G2-SID according to the present invention, Compound 57, which has four p-nitroaniline tail units and phenylacetic acid, a substrate for penicillin amidase [18, 19], as the trigger unit. When linked to the SID, the p-nitroaniline molecules are colorless. However, as is shown in FIG. 31, upon a single enzymatic cleavage by penicillin amidase the self-immolative reactions sequence is initiated, so as to yield four free p-nitroaniline molecules, which are characterized by yellow color in the release solution.

Compound 57 is obtained by synthesizing Compound 34 as is described in Example 6 and presented in FIG. 18, removing the BOC group by reaction with TFA and attaching to the resulting amine salt the penicillin amidase substrate.

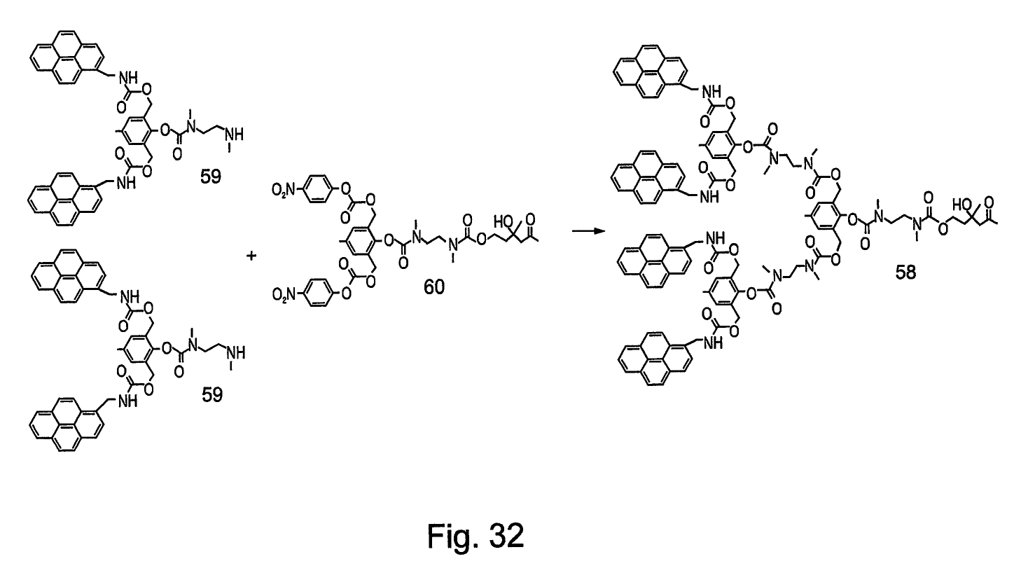

Similarly, as is presented in FIG. 32, a G2-SID enzymatic sensor Compound 58, which has four aminomethylpyrene tail units and the retro-aldol retro-Michael trigger unit described hereinabove, was synthesized. Compound 58 is relatively non-polar and simple to detect by UV-light.

As is shown in FIG. 32, Compound 58 was synthesized by attaching two molecules of the aminomethylpyrene-G1-SID Compound 59, prepared by deprotecting Compound 24 described hereinabove in Example 6 and FIG. 8, to the dicarbonate Compound 60, prepared by deprotecting Compound 45 (FIG. 23) and reacting the resulting amine salt with the retro-aldol retro-Michael substrate described hereinabove. Compound 59 was identified by ¹H-NMR and MS measurements.

### Example 11

### Activity Assays of Self-Immolative Dendrimers having Two Different Drugs as Tail Units and an Enzymatic Trigger Unit

In order to evaluate the efficacy of the self-immolative dendrimers of the present invention in simultaneously releasing two or more different drugs, and thus achieve synergism, the anti-proliferative activity of a G1-SID of the present invention, which has two different drug molecules as its tail units, has been tested.

As a representative example of such a G1-SID, model Compound 39 (see, FIG. 22), in which one doxorubicin molecule and one camptothecin molecule constitute the tail units (each denoted as “drug” in FIG. 22), a retro-aldol retro-Michael substrate constitutes the trigger unit, and 2,6-bishydroxymethyl-p-cresol serves as the base unit of the chemical linker, has been synthesized, according to the procedure described in Example 8, so as to yield the heterodimeric prodrug, Compound 61.

As is shown in FIG. 32, and is further described in detail hereinabove, in Compound 61, the doxorubicin is attached directly to the base unit via a carbamate linkage, whereas the camptothecin is attached to the base unit via an N,N′-dimethylethylenediamine spacer. As is further shown in FIG. 32, the chemical linker is attached to the trigger unit via a spacer that comprises two N,N′-dimethylethylenediamine, interrupted by a 2,4,6-trishydroxymethylphenol self-immolative unit, described hereinabove in Example 3. Such an extended spacer provides for reduction of the steric hindrance resulting from the two bulky drug tail units and hence renders the enzymatic trigger unit more accessible to the corresponding enzyme (38C2 antibody in this case).

FIG. 32 further presents the self-immolation of Compound 61, initiated by the catalytic antibody 38C2, to thereby simultaneously release the doxorubicin (DOX) and camptothecin (CPT) molecules.

The anti-proliferative activity of the heterodimeric prodrug Compound 61 was compared with the known mono-prodrugs of doxorubicin 55 and camptothecin 56, described hereinabove, using the cells-growth inhibition assay of Molt3 cell line, described hereinabove.

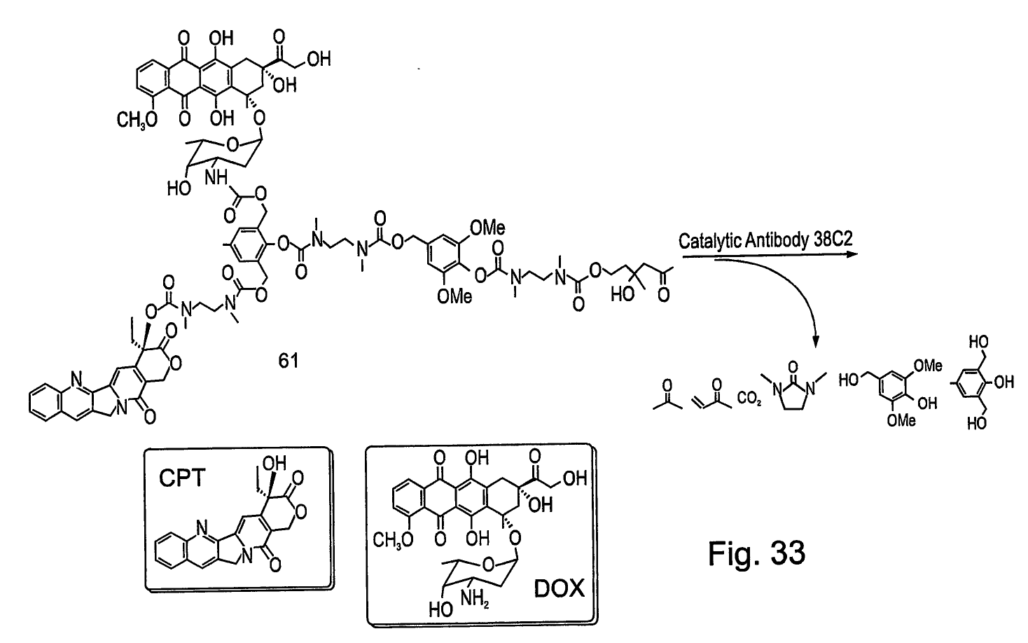

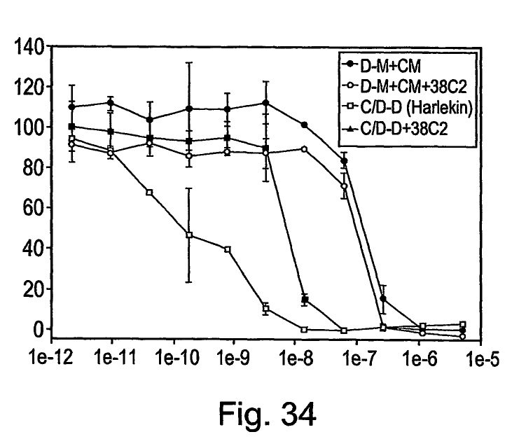

The results, presented in FIG. 33, show that similar IC50 values were obtained for a combination of the mono-prodrugs 55 and 56 and for the heterodimeric prodrug Compound 61. However, upon addition of the catalytic antibody 38C2, a substantially enhanced activation of the dimeric prodrug 61 was observed (about 100-folds), as compared with a moderate activation of the mono-prodrugs 55 and 56 (about 10-folds), thus clearly indicating a synergistic effect of the heterogenic G1-SID.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### LIST OF REFERENCES CITED BY NUMERALS

### (ADDITIONAL REFERENCES ARE CITED WITHIN THE TEXT)

1. Tomalia, D. A., and Frechet, J. M. J., Discovery of dendrimers and dendritic polymers: a brief historical perspective, in Journal of Polymer Science, Part A: Polymer Chemistry, Vol. 40, pp. 2719-2728 (2002).

2. Klajnert, B., and Bryszewska, M., Dendrimers: properties and applications, Acta Biochim Pol, 48, 199-208 (2001).

3. O. L. Padilla De Jesus, H. R. Ihre, L. Gagne, J. M. Frechet, F. C. Szoka, Jr., Bioconjug Chem 13 (2002) 453-61.

4. Kim, Y., and Zimmerman, S. C., Applications of dendrimers in bio-organic chemistry, Curr Opin Chem Biol, 2, 733-42. (1998).

5. Patri, A. K., Majoros, I. J., and Baker, J. R., Dendritic polymer macromolecular carriers for drug delivery, Curr Opin Chem Biol, 6, 466-71. (2002).

6. Stiriba, S.-E., Frey, H., and Haag, R., Dendritic polymers in biomedical applications: From potential to clinical use in diagnostics and therapy, in Angewandte Chemie, International Edition, Vol. 41, pp. 1329-1334 (2002).

7. Ihre, H. R., Padilla De Jesus, O. L., Szoka, F. C., Jr., and Frechet, J. M., Polyester dendritic systems for drug delivery applications: design, synthesis, and characterization, Bioconjug Chem, 13, 443-52. (2002).

8. Kojima, C., Kono, K., Maruyama, K., and Takagishi, T., Synthesis of polyamidoamine dendrimers having poly(ethylene glycol) grafts and their ability to encapsulate anticancer drugs, Bioconjug Chem, 11, 910-7. (2000).

9. R. Madec-Lougerstay, J.-C. Florent, C. Monneret, Journal of the Chemical Society, Perkin Transactions 1: Organic and Bio-Organic Chemistry 1999, p. 1369-1376.

10. Maeda, H., Wu, J., Sawa, T., Matsumura, Y., and Hori, K., Tumor vascular permeability and the EPR effect in macromolecular therapeutics: a review, J Controlled Release, 65, 271-84 (2000).

11. Satchi, R., Connors, T. A., and Duncan, R., PDEPT: polymer-directed enzyme prodrug therapy. I. HPMA copolymer-cathepsin B and PK1 as a model combination, Br J Cancer, 85, 1070-6. (2001).

12. Leu, Y. L., Roffler, S. R., and Chem, J. W., Design and synthesis of water-soluble glucuronide derivatives of camptothecin for cancer prodrug monotherapy and antibody-directed enzyme prodrug therapy (ADEPT), J Med Chem, 42,3623-8. (1999).

13. Hande, K. R., Etoposide: four decades of development of a topoisomerase II inhibitor, Eur. J. Cancer, 34, 1514-1521 (1998).

14. Wagner, J., Lerner, R. A., and Barbas, C. F., III., Efficient aldolase catalytic antibodies that use the enamine mechanism of natural enzymes, Science (Washington, D.C.), 270,1797-800 (1995).

15. Shabat, D., Lode, H., Pertl, U., reisfeld, R. A., Rader, C., Lerner, R. A., and Barbas, C. F., III, In vivo activity in a catalyic antibody-prodrug system: Antibody catalyzed etoposide prodrug activation for selective chemotherapy, Proc. Natl. Acad. Sci. U. S. A., 98,7528-33 (2001).

16. Shabat, D., Rader, C., List, B., Lerner, R. A., and Barbas, C. F., III, Multiple event activation of a generic prodrug trigger by antibody catalysis, in Proc. Natl. Acad. Sci. U. S. A., Vol. 96, pp. 6925-6930 (1999).

17. Satchi-Fainaro, R., Wrasildo, W., Lode, H. N. and Shabat D., Bioorg. Med. Chem., 10, 3023-9 (2002).

18. Forney, L. J., Wong, D. C., and Ferber, D. M., Selection of amidases with novel substrate specificities from penicillin amidase of Escherichia coli, Appl Environ Microbiol, 55, 2550-5. (1989).

19. Margolin, A. L., Svedas, V. K., and Berezin, I. V., Substrate specificity of penicillin amidase from E. coli, Biochim Biophys Acta, 616, 283-9. (1980).

1. A self-immolative dendrimer comprising a cleavable trigger unit, a plurality of tail units and at least one self-immolative chemical linker linking between said trigger unit and said tail units, said trigger unit and said at least one self-immolative chemical linker being such that upon cleavage of said trigger unit, said at least one self-immolative chemical linker self-immolates, thereby releasing said tail units.

2. The self-immolative dendrimer of claim 1, wherein said tail units comprise at least two functional moieties, said at least two functional moieties being the same or different.

3. The self-immolative dendrimer of claim 1, further comprising at least one self-immolative spacer.

4. The self-immolative dendrimer of claim 3, wherein said spacer linking said trigger unit and said at least one self-immolative chemical linker.

5. The self-immolative dendrimer of claim 3, wherein said at least one spacer linking at least one of said tail units and at least one of said at least one chemical linker.

6. The self-immolative dendrimer of claim 3, wherein said trigger unit, said at least one spacer and said at least one self-immolative chemical linker being such that upon cleavage of said trigger unit, said at least one self-immolative chemical linker and said at least one spacer self-immolate to thereby release said tail units.

7. The self-immolative dendrimer of claim 1, wherein said cleavable trigger unit is selected from the group consisting of a photo-labile trigger unit, a chemically removable trigger unit, a hydrolysable trigger unit and a biodegradable trigger unit.

8. The self-immolative dendrimer of claim 7, wherein said biodegradable trigger unit is an enzymatically cleavable trigger unit.

9. The self-immolative dendrimer of claim 2, wherein said functional moieties comprise at least one therapeutically active agent.

10. The self-immolative dendrimer of claim 2, wherein said functional moieties comprise at least two therapeutically active agents.

11. The self-immolative dendrimer of claim 2, wherein said at least two therapeutically active agents are synergistic.

12. The self-immolative dendrimer of claim 2, wherein said functional moieties comprise at least one diagnostic agent.

13. The self-immolative dendrimer of claim 9 wherein each of said therapeutically active agents is selected from the group consisting of an anti-proliferative agent, an anti-inflammatory agent, an antibiotic, an anti-viral agent, an anti-hypertensive agent, a chemosensitizing agent and a combination thereof.

14. The self-immolative dendrimer of claim 13, wherein said anti-proliferative agent is a chemotherapeutic agent.

15. The self-immolative dendrimer of claim 12, wherein said at least one diagnostic agent is selected from the group consisting of a signal generator agent, a single absorber agent and a combination thereof.

16. The self-immolative dendrimer of claim 1, wherein said self-immolative chemical linker has a general formula selected from the group consisting of Formula Ia and Formula Ib: 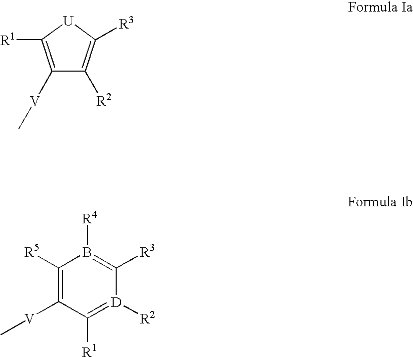
wherein:
V is O, S, PR⁶or NR⁷;
U is O, S or NR⁸;
B and D are each independently a carbon atom or a nitrogen atom;
R¹, R², R³, R⁴ and R⁵ are each independently 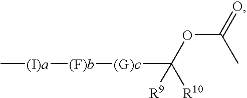
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
whereas:
a, b and c are each independently as integer of 0 to 5; and
I, F and G are each independently —R¹¹C═CR¹²— or —C≡C—, where each of R¹¹ and R¹² is independently -hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R¹, R² and R³ in Formula Ia and of R¹, R², R³, R⁴ and R⁵ in Formula Ib are said

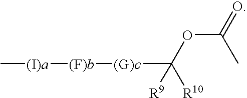

17. The self-immolative dendrimer of claim 16, wherein said self-immolative chemical linker has the general Formula Ib.

18. The self-immolative dendrimer of claim 17, wherein:
V is O or S;
each of B and D is a carbon atom;
each of R², R³ and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹ and R⁵ is said

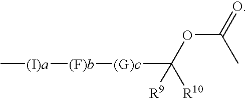

19. The self-immolative dendrimer of claim 18, wherein:
each of R², R³ and R⁴ is independently hydrogen or alkyl;
each of a, b and c equal 0; and
each of R⁹ and R¹⁰ is independently hydrogen or alkyl.

20. The self-immolative dendrimer of claim 17, wherein
V is O or S;
each of B and D is a carbon atom;
each of R² and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹, R³ and R⁵ is said

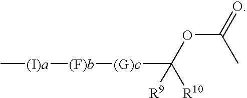

21. The self-immolative dendrimer of claim 20, wherein:
each of R² and R⁴ is independently hydrogen or alkyl;
each of a, b and c equal 0; and
each of R⁹ and R¹⁰ is independently hydrogen or alkyl.

22. The self-immolative dendrimer of claim 3, wherein said self-immolative spacer has a general formula selected from the group consisting of Formula IIa, Formula IIb, Formula IIc, Formula IId: 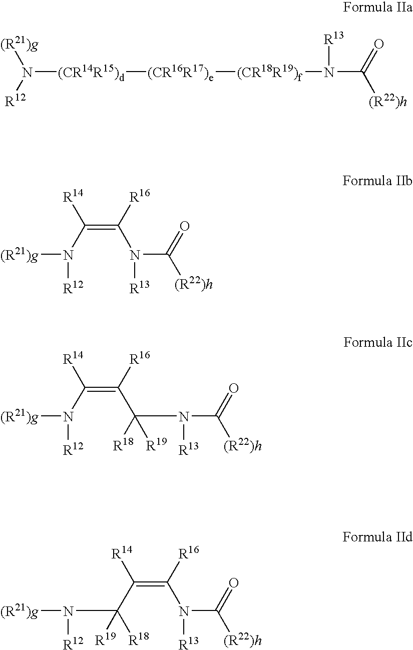
and a combination thereof,
wherein:
d, e, f, g and h are each independently an integer from 0 to 3, provided that d+e+f≧2;
R¹² and R¹³ are each independently hydrogen, alkyl or cycloalkyl;
R¹⁴, R¹⁵, R¹⁶, R¹⁷, R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate;
R²¹ and R²² each independently has a general formula selected from the group consisting of Formula VIIa and Formula VIIb: 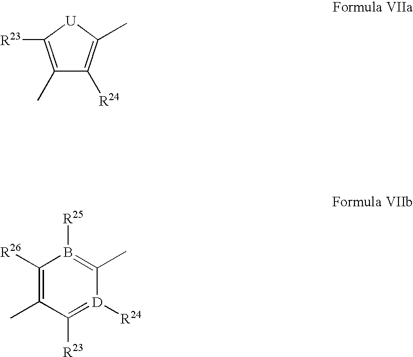
wherein:
U is O, S or NR²⁹;
B and D are each independently a carbon atom or a nitrogen atom;
R²³, R²⁴, R²⁵ and R²⁶ are each independently 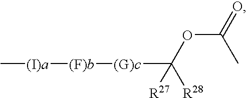
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R²³, R²⁴, R²⁵ and R²⁶ being connected to one another to form an aromatic or aliphatic cyclic structure;
whereas:
a, b and c are each independently as integer of 0 to 5; and
I, F and G are each independently —R³⁰C═CR³¹— or —C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R²³ and R²⁴ in Formula VIIa and of R²³, R²⁴, R³⁵ and R²⁶ in Formula VIIb are said

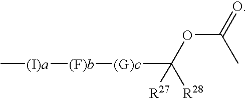

23. The self-immolative dendrimer of claim 22, wherein said self-immolative spacer has the general Formula IIa.

24. The self-immolative dendrimer of claim 1, being between a first and a tenth generation dendrimer.

25. The self-immolative dendrimer of claim 1, having between 2 and 5 ramifications in each generation.

26. The self-immolative dendrimer of claim 2, wherein said trigger unit is an enzymatically cleavable trigger unit and said functional moieties comprise at least one therapeutically active agent.

27. The self-immolative dendrimer of claim 26, wherein said at least one therapeutically active agent comprises at least one chemotherapeutic agent.

28. The self-immolative dendrimer of claim 2, wherein said trigger unit is an enzymatically cleavable trigger unit and said functional moieties comprise at least two therapeutically active agents.

29. The self-immolative dendrimer of claim 2, wherein said at least two therapeutically active agents are synergistic.

30. The self-immolative dendrimer of claim 2, wherein said trigger unit is an enzymatically cleavable trigger unit and said functional moieties comprise at least one diagnostic agent.

31. The self-immolative dendrimer of claim 30, wherein said at least one diagnostic agent is selected from the group consisting of a signal generator agent, a signal absorber agent and a combination thereof.

32. The self-immolative dendrimer of claim 2, wherein said trigger unit is a photo-labile trigger unit and said functional moieties comprise at least one diagnostic agent.

33. The self-immolative dendrimer of claim 2, wherein said trigger unit is a hydrolyzable trigger unit and said functional moieties comprise at least one agrochemical.

34. The self-immolative dendrimer of claim 2, wherein said trigger unit is a chemically removable trigger unit and said functional moieties comprise at least one diagnostic agent.

35. A self-immolative dendrimer having a general Formula III:
Q-Ai-Z⁰[(X₀)j(Y₀)k]-Z¹[(X₁)l(Y₁)m]- . . . -Zⁿ[(Xn)p(Yn)r]-Zⁿ⁺¹[W] Formula III
wherein:
n is an integer from 0 to 20;
each of i, j, k, l, m, p and r is independently an integer of 0 to 10;
Q is a cleavable trigger unit;
A is a first self-immolative spacer;
Z is an integer of between 2 and 6, representing the ramification number of the dendrimer;
X is a self-immolative chemical linker;
Y is a second self-immolative spacer; and
W is a tail unit,
whereas, when n equals 0, each of l, m, p and r equals 0; and
when n equals 1, each of p and r equals 0.

36. The self-immolative dendrimer of claim 35, wherein said Zⁿ⁺¹[W] comprise at least two functional moieties, said functional moieties being the same or different.

37. The self-immolative dendrimer of claim 35, wherein Z equals 2 or 3.

38. The self-immolative dendrimer of claim 35, wherein n is an integer of 0 to 10.

39. The self-immolative dendrimer of claim 35, wherein said cleavable trigger unit Q is selected from the group consisting of a photo-labile trigger unit, a chemically removable trigger unit, a hydrolyzable trigger unit and a biodegradable trigger unit.

40. The self-immolative dendrimer of claim 39, wherein said biodegradable trigger unit is an enzymatically cleavable trigger unit.

41. The self-immolative dendrimer of claim 36, wherein said functional moieties W comprise at least one therapeutically active agent.

42. The self-immolative dendrimer of claim 36, wherein said functional moieties W comprise at least one diagnostic agent.

43. The self-immolative dendrimer of claim 41, wherein said at least one therapeutically active agent is selected from the group consisting of an anti-proliferative agent, an anti-inflammatory agent, an antibiotic, an anti-viral agent, an anti-hypertensive agent and combinations thereof.

44. The self-immolative dendrimer of claim 43, wherein said anti-proliferative agent is a chemotherapeutic agent.

45. The self-immolative dendrimer of claim 42, wherein said at least one diagnostic agent is selected from the group consisting of a signal generator agent, a single absorber agent and a combination thereof.

46. The self-immolative dendrimer of claim 35, wherein each of said self-immolative chemical linkers X₀-Xn independently has a general formula selected from the group consisting of Formula Ia and Formula Ib: 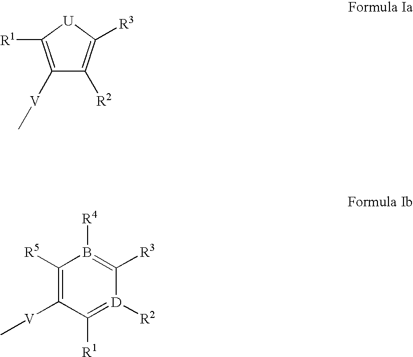
wherein:
V is O, S, PR⁶ or NR⁷;
U is O, S or NR⁸;
B and D are each independently a carbon atom or a nitrogen atom;
R¹, R², R³, R⁴ and R⁵ are each independently 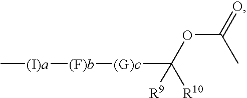
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
whereas:
a, b and c are each independently as integer of 0 to 5; and
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R¹, R² and R³ in Formula Ia and of R¹, R², R³, R⁴ and R⁵ in Formula Ib are said

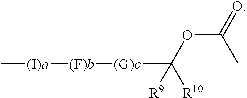

47. The self-immolative dendrimer of claim 46, wherein each of said self-immolative chemical linkers X₀-Xn has the general Formula Ib.

48. The self-immolative dendrimer of claim 47, wherein:
V is O or S;
each of B and D is a carbon atom;
each of R², R³ and R⁴ is independently hydrogen, alkyl, aryl, cycloalkyl, heterocycloalkyl, heteroaryl, alkoxy, hydroxy, amino, nitro, halo, trihalomethyl, cyano, amido, cyclic alkylamino, imidazolyl, alkylpiperazinyl, morpholino, thio, thioether, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹ and R⁵ is said

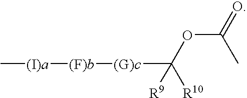

49. The self-immolative dendrimer of claim 48, wherein:
each of R², R³ and R⁴ is independently hydrogen or alkyl;
each of a, b and c equal 0; and
each of R⁹ and R¹⁰ is independently hydrogen or alkyl.

50. The self-immolative dendrimer of claim 47, wherein
V is O or S;
each of B and D is a carbon atom;
each of R² and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹, R³ and R⁵ is said

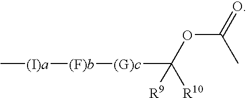

51. The self-immolative dendrimer of claim 50, wherein:
each of R² and R⁴ is independently hydrogen or alkyl;
each of a, b and c equal 0; and
each of R⁹ and R¹⁰ is independently hydrogen or alkyl.

52. The self-immolative dendrimer of claim 35, wherein each of said first self-immolative spacer A and said self-immolative spacers Y₀-Yn independently has a general formula selected from the group consisting of Formula IIa, Formula IIb, Formula IIc and Formula IId: 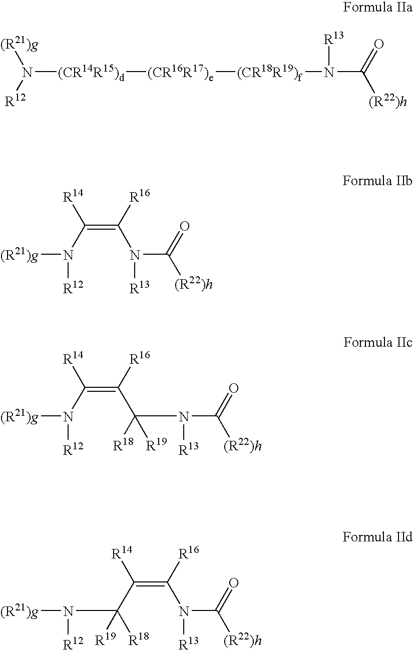
and a combination thereof,
wherein:
d, e, f, g and h and f are each independently an integer from 0 to 3, provided that d+e+f≧2;
R¹² and R¹³ are each independently hydrogen or alkyl;
R¹⁴, R¹⁵, R¹⁶, R¹⁷, R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate;
R²¹ and R²² are each independently has a general formula selected from the group consisting of Formula VIIa and Formula VIIb: 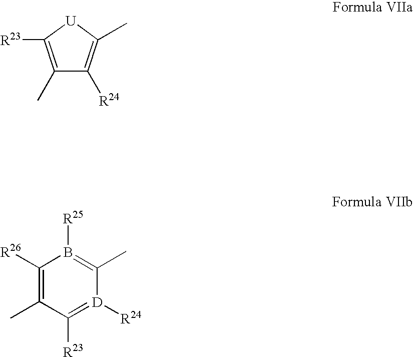
wherein:
U is O, S or NR²⁹;
B and D are each independently a carbon atom or a nitrogen atom;
R²³, R²⁴, R²⁵ and R²⁶ are each independently 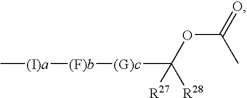
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R²³, R²⁴, R²⁵ and R²⁶ being connected to one another to form an aromatic or aliphatic cyclic structure;
whereas:
a, b and c are each independently as integer of 0 to 5; and
I, F and G are each independently —R³⁰C═CR³¹— or —C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino tetrazole, carboxy, carboxylate, sulfoxy, sulfonate, sulfonyl, sulfixy, sulfinate, sulfinyl, phosphonooxy or phosphate, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R²³ and R²⁴ in Formula VIIa and of R²³, R²⁴, R³⁵ and R²⁶ in Formula VIIb are said

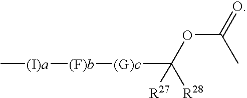

53. The self-immolative dendrimer of claim 52, wherein each of said first self-immolative spacer A and said self-immolative spacers Y₀-Yn independently has the general Formula IIa.

54. A pharmaceutical composition comprising, as an active ingredient, the self-immolative dendrimer of claim 2 and a pharmaceutically acceptable carrier.

55. The pharmaceutical composition of claim 54, packaged in a packaging material and identified in print, in or on said packaging material, for use in the treatment of a disease or disorder selected from the group consisting of a proliferative disease or disorder, an inflammatory disease or disorder, a bacterial disease or disorder, a viral disease or disorder and a hypertensive disease or disorder.

56. The pharmaceutical composition of claim 54, packaged in a packaging material and identified in print, in or on said packaging material, for use in a diagnosis.

57. The pharmaceutical composition of claim 54, wherein said self-immolative dendrimer further comprises at least one self-immolative spacer.

58. The pharmaceutical composition of claim 57, wherein said spacer linking said trigger unit and said at least one self-immolative chemical linker.

59. The pharmaceutical composition of claim 57, wherein said at least one spacer linking at least one of said functional moieties and at least one of said at least one chemical linker.

60. The pharmaceutical composition of claim 57, wherein said trigger unit, said at least one spacer and said at least one self-immolative chemical linker being such that upon cleavage of said trigger unit, said at least one self-immolative chemical linker and said at least one spacer self-immolate to thereby release said functional moieties

61. The pharmaceutical composition of claim 54, wherein said cleavable trigger unit is selected from the group consisting of a photo-labile trigger unit, a chemically removable trigger unit, a hydrolizable trigger unit and a biodegradable trigger unit.

62. The pharmaceutical composition of claim 61, wherein said biodegradable trigger unit is an enzymatically cleavable trigger unit.

63. The pharmaceutical composition of claim 54, wherein said functional moieties comprise at least one therapeutically active agent.

64. The pharmaceutical composition of claim 54, wherein said functional moieties comprise at least two therapeutically active agents.

65. The pharmaceutical composition of claim 64, wherein said at least two therapeutically active agents are synergistic.

66. The pharmaceutical composition of claim 54, wherein said functional moieties comprise at least one diagnostic agent.

67. The pharmaceutical composition of claim 63, wherein said at least one therapeutically active agent is selected from the group consisting of an anti-proliferative agent, an anti-inflammatory agent, an antibiotic, an anti-viral agent, an anti-hypertensive agent, a chemosensitizing agent and a combination thereof.

68. The pharmaceutical composition of claim 67, wherein said anti-proliferative agent is a chemotherapeutic agent.

69. The pharmaceutical composition of claim 66, wherein said diagnostic agent is selected from the group consisting of a signal generator agent, a single absorber agent and a combination thereof.

70. The pharmaceutical composition of claim 54, wherein said self-immolative chemical linker has a general formula selected from the group consisting of Formula Ia and Formula Ib: 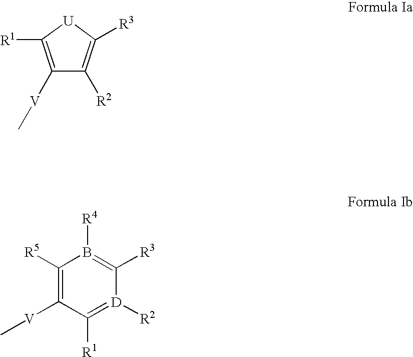
wherein:
V is O, S, PR⁶ or NR⁷;
U is O, S or NR⁸;
B and D are each independently a carbon atom or a nitrogen atom;
R¹, R², R³, R⁴ and R⁵ are each independently 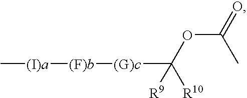
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
whereas:
a, b and c are each independently as integer of 0 to 5; and
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R¹, R² and R³ in Formula Ia and of R¹, R², R³, R⁴ and R⁵ in Formula Ib are said

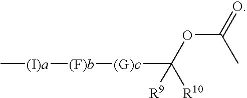

71. The pharmaceutical composition of claim 70, wherein said self-immolative chemical linker has the general Formula Ib.

72. The pharmaceutical composition of claim 71, wherein:
V is O or S;
each of B and D is a carbon atom;
each of R², R³ and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹ and R⁵ is said

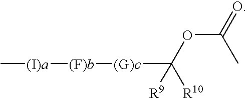

73. The pharmaceutical composition of claim 72, wherein:
each of R², R³ and R⁴ is independently hydrogen or alkyl;
each of a, b and c equal 0; and
each of R⁹ and R¹⁰ is independently hydrogen or alkyl.

74. The pharmaceutical composition of claim 71, wherein
V is O or S;
each of B and D is a carbon atom;
each of R² and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹, R³ and R⁵ is said

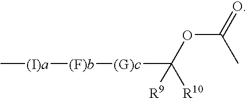

75. The pharmaceutical composition of claim 74, wherein:
each of R² and R⁴ is independently hydrogen or alkyl;
each of a, b and c equal 0; and
each of R⁹ and R¹⁰ is independently hydrogen or alkyl.

76. The pharmaceutical composition of claim 57, wherein said self-immolative spacer has a general formula selected from the group consisting of Formula IIa, Formula IIb, Formula IIc and Formula IId: 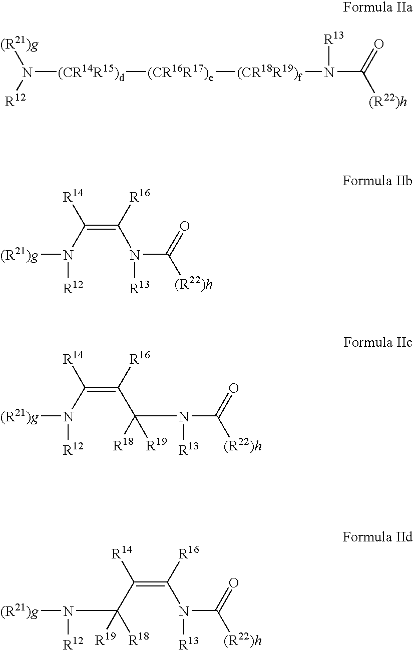
and a combination thereof,
wherein:
d, e, f, g and h and f are each independently an integer from 0 to 3, provided that d+e+f≧2;
R¹² and R¹³ are each independently hydrogen or alkyl;
R¹⁴, R¹⁵, R¹⁶, R¹⁷, R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate;
R²¹ and R²² are each independently has a general formula selected from the group consisting of Formula VIIa and Formula VIIb: 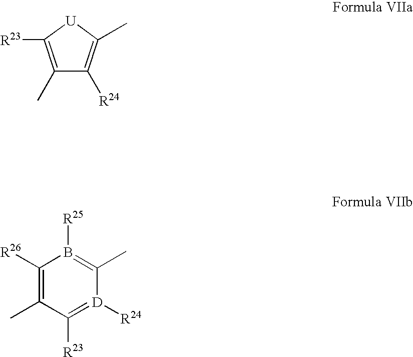
wherein:
U is O, S or NR²⁹;
B and D are each independently a carbon atom or a nitrogen atom;
R²³, R²⁴, R²⁵ and R²⁶ are each independently 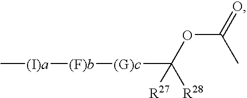
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R²³, R²⁴, R²⁵ and R²⁶ being connected to one another to form an aromatic or aliphatic cyclic structure;
whereas:
a, b and c are each independently as integer of 0 to 5; and
I, F and G are each independently —R³⁰C═CR³¹— or —C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R²³ and R²⁴ in Formula VIIa and of R²³, R²⁴, R³⁵ and R²⁶ in Formula VIIb are said

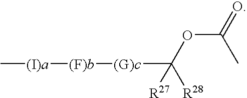

77. The pharmaceutical composition of claim 76, wherein said self-immolative spacer has the general Formula IIa.

78. The pharmaceutical composition of claim 54, wherein said self-immolative dendrimer is between a first and a tenth generation dendrimer.

79. The pharmaceutical composition of claim 54, wherein said self-immolative dendrimer has between 2 and 5 ramifications in each generation.

80. The pharmaceutical composition of claim 54, wherein said trigger unit is an enzymatically cleavable trigger unit and said functional moieties comprise at least one therapeutically active agent.

81. The pharmaceutical composition of claim 80, wherein said at least one therapeutically active agent comprises at least one chemotherapeutic agent.

82. The pharmaceutical composition of claim 54, wherein said trigger unit is an enzymatically cleavable trigger unit and said functional moieties comprise at least one diagnostic agent.

83. The pharmaceutical composition of claim 82, wherein said at least one diagnostic agent is selected from the group consisting of a signal generator agent, a signal absorber agent and a combination therefore.

84. A pharmaceutical composition comprising, as an active ingredient, the self-immolative dendrimer of claim 36 and a pharmaceutically acceptable carrier.

85. The pharmaceutical composition of claim 84, wherein Z equals 2 or 3.

86. The pharmaceutical composition of claim 84, wherein n is an integer of 0 to 10.

87. The pharmaceutical composition of claim 84, wherein said cleavable trigger unit Q is selected from the group consisting of a photo-labile trigger unit, a chemically removable trigger unit, a hydrolysable trigger unit and a biodegradable trigger unit.

88. The pharmaceutical composition of claim 87, wherein said biodegradable trigger unit is an enzymatically cleavable trigger unit.

89. The pharmaceutical composition of claim 84, wherein said functional moieties W comprise at least one therapeutically active agent.

90. The pharmaceutical composition of claim 84, wherein said functional moieties W comprise at least one diagnostic agent.

91. The pharmaceutical composition of claim 89, wherein said at least one therapeutically active agent is selected from the group consisting of an anti-proliferative agent, an anti-inflammatory agent, an antibiotic, an anti-viral agent, an anti-hypertensive agent, a chemosensitizing agent and combinations thereof.

92. The pharmaceutical composition of claim 91, wherein said anti-proliferative agent is a chemotherapeutic agent.

93. The pharmaceutical composition of claim 90, wherein said at least one diagnostic agent is selected from the group consisting of a signal generator agent, a single absorber agent and a combination thereof.

94. The pharmaceutical composition of claim 84, wherein each of said self-immolative chemical linkers X₀-Xn independently has a general formula selected from the group consisting of Formula Ia and Formula Ib: 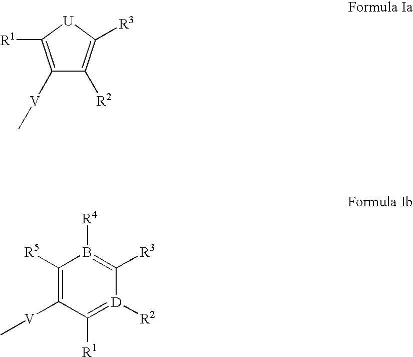
wherein:
V is O, S, PR⁶ or NR⁷;
U is O, S or NR⁸;
B and D are each independently a carbon atom or a nitrogen atom;
R¹, R², R³, R⁴ and R⁵ are each independently 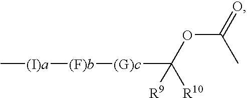
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
whereas:
a, b and c are each independently as integer of 0 to 5; and
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R¹, R² and R³ in Formula Ia and of R¹, R², R³, R⁴ and R⁵ in Formula Ib are said

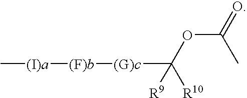

95. The pharmaceutical composition of claim 94, wherein each of said self-immolative chemical linkers X₀-Xn has the general Formula Ib.

96. The pharmaceutical composition of claim 95, wherein:
V is O or S;
each of B and D is a carbon atom;
each of R², R³ and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹ and R⁵ is said

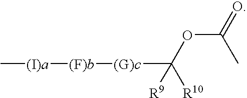

97. The pharmaceutical composition of claim 96, wherein:
each of R², R³ and R⁴ is independently hydrogen or alkyl;
each of a, b and c equal 0; and
each of R⁹ and R¹⁰ is independently hydrogen or alkyl.

98. The pharmaceutical composition of claim 95, wherein
V is O or S;
each of B and D is a carbon atom;
each of R² and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹, R³ and R⁵ is said

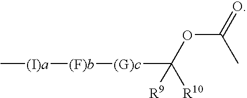

99. The pharmaceutical composition of claim 98, wherein:
each of R² and R⁴ is independently hydrogen or alkyl;
each of a, b and c equal 0; and
each of R⁹ and R¹⁰ is independently hydrogen or alkyl.

100. The pharmaceutical composition of claim 84, wherein each of said first self-immolative spacer A and said self-immolative spacers Y₀-Yn independently has a general formula selected from the group consisting of Formula IIa, Formula IIb, Formula IIc and Formula IId: 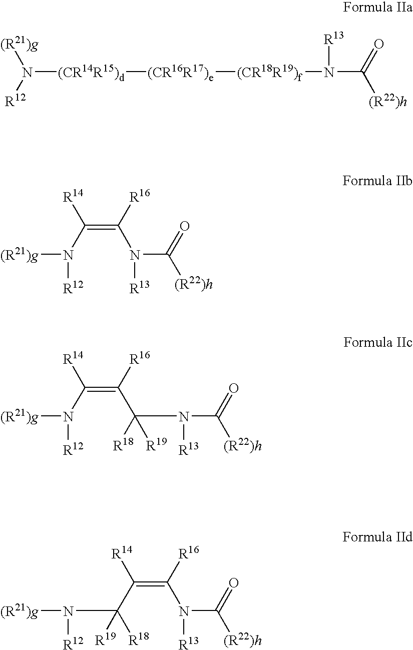
and a combination thereof,
wherein:
d, e, f, g and h and f are each independently an integer from 0 to 3, provided that d+e+f≧2;
R¹² and R¹³ are each independently hydrogen or alkyl;
R¹⁴, R¹⁵, R¹⁶, R¹⁷, R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate;
R²¹ and R²² are each independently has a general formula selected from the group consisting of Formula VIIa and Formula VIIb: 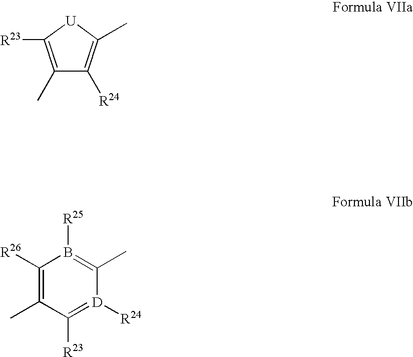
wherein:
U is O, S or NR²⁹;
B and D are each independently a carbon atom or a nitrogen atom;
R²³, R²⁴, R²⁵ and R²⁶ are each independently 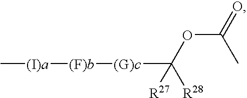
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R²³, R²⁴, R²⁵ and R²⁶ being connected to one another to form an aromatic or aliphatic cyclic structure;
whereas:
a, b and c are each independently as integer of 0 to 5; and
I, F and G are each independently —R³⁰C═CR³¹— or —C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R²³ and R²⁴ in Formula VIIa and of R²³, R²⁴, R³⁵ and R²⁶ in Formula VIIb are said

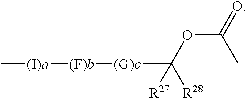

101. The pharmaceutical composition of claim 100, wherein each of said first self-immolative spacer A and said self-immolative spacers Y₀-Yn independently has the general Formula IIa.

102. An agricultural composition, comprising, as an active ingredient, the self-immolative dendrimer of claim 33, and an agricultural acceptable carrier.

103. A method of treating a disorder or disease selected from the group consisting of a proliferative disease or disorder, an inflammatory disease or disorder, a bacterial disease or disorder, a viral disease or disorder and a hypertensive disease or disorder in a subject in need thereof, the method comprising administering to the subject a therapeutically effective amount of the self-immolative dendrimer of claim 9, 10 or 11.

104. The method of claim 103, wherein said self-immolative dendrimer further comprises at least one self-immolative spacer.

105. The method of claim 104, wherein said spacer linking said trigger unit and said at least one self-immolative chemical linker.

106. The method of claim 104, wherein said at least one spacer linking at least one of said functional moieties and at least one of said at least one chemical linker.

107. The method of claim 104, wherein said trigger unit, said at least one spacer and said at least one self-immolative chemical linker being such that upon cleavage of said trigger unit, said at least one self-immolative chemical linker and said at least one spacer self-immolate to thereby release said functional moieties.

108. The method of claim 103, wherein said cleavable trigger unit is an enzymatically cleavable trigger unit.

109. The method of claim 103, wherein said at least one therapeutically active agent is selected from the group consisting of an anti-proliferative agent, an anti-inflammatory agent, an antibiotic, an anti-viral agent, an anti-hypertensive agent, a chemosensitizing agent and a combination thereof.

110. The method of claim 109, wherein said anti-proliferative agent is a chemotherapeutic agent.

111. The method of claim 103, wherein said self-immolative chemical linker has a general formula selected from the group consisting of Formula Ia and Formula Ib: 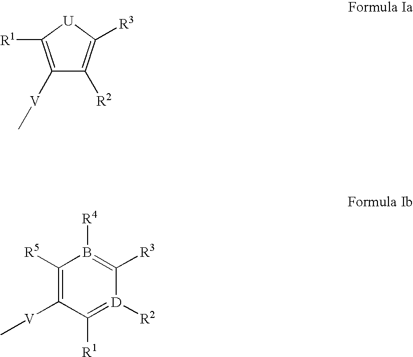
wherein:
V is O, S, PR⁶ or NR⁷;
U is O, S or NR⁸;
B and D are each independently a carbon atom or a nitrogen atom;
R¹, R², R³, R⁴ and R⁵ are each independently 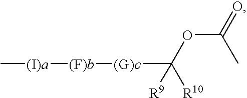
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
whereas:
a, b and c are each independently as integer of 0 to 5; and
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R¹, R² and R³ in Formula Ia and of R¹, R², R³, R⁴ and R⁵ in Formula Ib are said

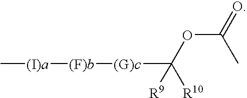

112. The method of claim 111, wherein said self-immolative chemical linker has the general Formula Ib.

113. The method of claim 112, wherein:
V is O or S;
each of B and D is a carbon atom;
each of R², R³ and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹ and R⁵ is said

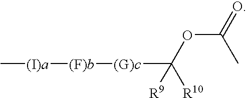

114. The method of claim 113, wherein:
each of R², R³ and R⁴ is independently hydrogen or alkyl;
each of a, b and c equal 0; and
each of R⁹ and R¹⁰ is independently hydrogen or alkyl.

115. The method of claim 112, wherein
V is O or S;
each of B and D is a carbon atom;
each of R² and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹, R³ and R⁵ is said

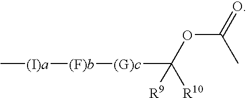

116. The method of claim 115, wherein:
each of R² and R⁴ is independently hydrogen or alkyl;
each of a, b and c equal 0; and
each of R⁹ and R¹⁰ is independently hydrogen or alkyl.

117. The method of claim 104, wherein said self-immolative spacer has a general formula selected from the group consisting of Formula IIa, Formula IIb, Formula IIc and Formula IId: 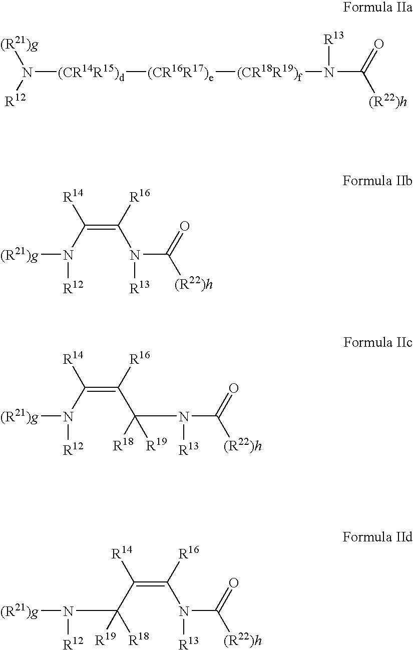
and a combination thereof,
wherein:
d, e, f, g and h and f are each independently an integer from 0 to 3, provided that d+e+f≧2;
R¹² and R¹³ are each independently hydrogen or alkyl;
R¹⁴, R¹⁵, R¹⁶, R¹⁷, R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate;
R²¹ and R²² are each independently has a general formula selected from the group consisting of Formula VIIa and Formula VIIb: 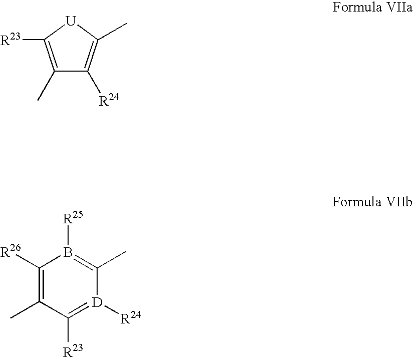
wherein:
U is O, S or NR²⁹;
B and D are each independently a carbon atom or a nitrogen atom;
R²³, R²⁴, R²⁵ and R²⁶ are each independently 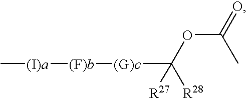
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R²³, R²⁴, R²⁵ and R²⁶ being connected to one another to form an aromatic or aliphatic cyclic structure;
whereas:
a, b and c are each independently as integer of 0 to 5; and
I, F and G are each independently —R³⁰C═CR³¹— or —C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R²³ and R²⁴ in Formula VIIa and of R²³, R²⁴, R³⁵ and R²⁶ in Formula VIIb are said

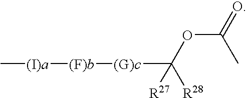

118. The method of claim 117, wherein said self-immolative spacer has the general Formula IIa.

119. A method of determining a concentration of an enzyme, the method comprising contacting said enzyme with the self-immolative dendrimer of claim 30.

120. The method of claim 87, wherein said contacting is effected in vitro.

121. The method of claim 87, wherein said contacting is effected in vivo.

122. A method of determining a concentration of a chemical reagent, the method comprising contacting said chemical reagent with the self-immolative dendrimer of claim 34.

123. A method of synthesizing a first generation self-immolative dendrimer of claim 1, the method comprising:
(a) providing a first compound having said self-immolative chemical linker being linked to said cleavable trigger unit and to at least two first reactive groups; and
(b) coupling said first compound with at least two equivalents of at least one second compound, thereby generating said first generation self-immolative dendrimer of claim 1 having a cleavable trigger unit as its core, at least two residues of said second compound as its tail units and a self-immolative chemical linker linking therebetween.

124. The method of claim 123, wherein said self-immolative chemical linker is linked to said cleavable trigger unit via a self-immolative spacer, the method further comprising, prior to (a):
(c) providing a third compound having said self-immolative chemical linker being linked to said at least two first reactive groups and to a second reactive group;
(d) coupling said third compound with said self-immolative spacer, to thereby provide a forth compound having said self-immolative chemical linker being linked to said at least two first reactive groups and to said self-immolative spacer; and
(e) coupling said forth compound with said cleavable trigger unit, to thereby provide said first compound.

125. The method of claim 123, wherein each of said first reactive groups comprises a carbonate group.

126. The method of claim 123, wherein said second compound comprises a free amino group.

127. The method of claim 123, wherein at least one of said tail units is linked to said chemical linker via a self-immolative spacer, the method further comprising, prior to (b):
(f) providing at least one second compound having a self-immolative spacer linked thereto.

128. The method of claim 124, wherein said second reactive group is selected from the group consisting of a hydroxyl, a thiol and an amine.

129. The method of claim 123, wherein said first compound has a general formula selected from the group consisting of Formula IVa and Formula IVb: 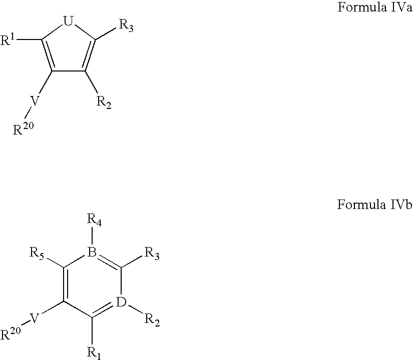
wherein:
V is O, S, PR⁶ or NR⁷;
U is O, S or NR⁸;
B and D are each independently a carbon atom or a nitrogen atom;
R¹, R², R³, R⁴ and R⁵ are each independently 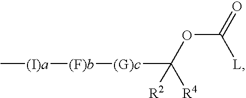
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
whereas:
a, b and c are each independently as integer of 0 to 5;
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
L is a leaving group;
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate; and
R²⁰ is said cleavable trigger unit or a self-immolative spacer terminating with said cleavable trigger unit,
provided that at least two of R¹, R² and R³ in Formula Ia and of R¹, R², R³, R⁴ and R⁵ in Formula Ib are said

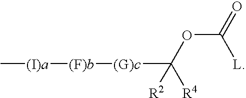

130. A method of synthesizing a first generation of the self-immolative dendrimer of claim 1, the method comprising:
(a) providing a first compound having a self-immolative chemical linker being linked to at least two tail units and to a first reactive group; and
(b) coupling said first compound with said cleavable trigger unit.

131. The method of claim 123, wherein said self-immolative chemical linker is linked to said cleavable trigger unit via a self-immolative spacer, the method further comprising, prior to (b):
(c) coupling said first compound with said self-immolative spacer.

132. The method of claim 130, wherein each of said tail units is linked to said chemical linker via a carbamate bond.

133. The method of claim 132, wherein said tail units are derived from at least one second compound having a free amino group.

134. The method of claim 130, wherein at least one of said tail units is linked to said chemical linker via a self-immolative spacer, the method further comprising, prior to (a):
(f) providing at least one third compound having a self-immolative spacer linked thereto; and
(g) coupling at least two equivalents of said at least one third compound with said self-immolative chemical linker.

135. The method of claim 130, wherein said first reactive group is selected from the group consisting of a hydroxyl, a thiol and an amine.

136. The method of claim 130, wherein said first compound has a general formula selected from the group consisting of Formula IVa and Formula IVb: 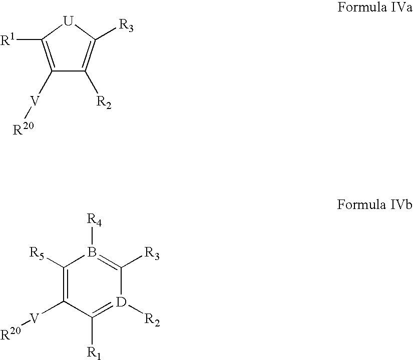
wherein:
V is O, S, PR⁶ or NR⁷;
U is O, S or NR⁸;
B and D are each independently a carbon atom or a nitrogen atom;
R¹, R², R³, R⁴ and R⁵ are each independently 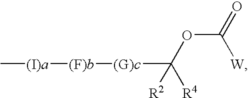
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
whereas:
a, b and c are each independently as integer of 0 to 5;
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
W is one of said at least two tail unit;
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate; and
R²⁰ is hydrogen, alkyl or cycloalkyl,
provided that at least two of R¹, R² and R³ in Formula Ia and of R¹, R², R³, R⁴ and R⁵ in Formula Ib are said

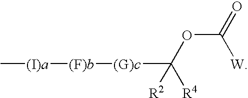

137. A method of synthesizing a Nth generation self-immolative dendrimer of claim 1, wherein N is an integer greater than 1, the method comprising:
(a) providing a (N−1)th generation self-immolative dendrimer including a first self-immolative chemical linker being linked to a first reactive group, (N−1) (N−1)th self-immolative chemical linkers each being linked to at least two second reactive groups, and a plurality of self-immolative chemical linkers linking therebetween;
(b) providing a Nth compound having a Nth self-immolative chemical linker being linked to a (N+1)th reactive group and to at least two (N+2)th reactive groups;
(c) coupling at least 2(N−1) equivalents of said Nth compound to said (N−1)th generation self-immolative dendrimer, to thereby provide a Nth generation self-immolative dendrimer having at least 2N (N+2)th reactive groups as its tail units and said first reactive group being linked to said first chemical linker; and
(d) coupling said Nth generation self-immolative dendrimer having at least 2N (N+2)th reactive groups as its tail units with at least 2N equivalents of at least one (N+1)th compound, to thereby provide said Nth generation self-immolative dendrimer having 2N tail units and a first reactive group as its core.

138. The method of claim 137, wherein said first reactive group is said cleavable trigger unit.

139. The method of claim 137, wherein said first reactive group is selected from the group consisting of hydroxyl, thiol and amine, the method further comprising, prior to (a) or after (d):
(e) coupling said first reactive group with said cleavable trigger unit.

140. The method of claim 138, wherein said first self-immolative chemical linker is linked to said cleavable trigger unit via a self-immolative spacer, the method further comprising, prior to (a):
(f) coupling said first self-immolative chemical linker with said self-immolative spacer.

141. The method of claim 139, wherein said first self-immolative chemical linker is linked to said cleavable trigger unit via a self-immolative spacer, the method further comprising, prior to (e):
(g) coupling said first reactive group with said self-immolative spacer.

142. The method of claim 137, wherein each of said second and (N+2)th reactive groups comprises a carbonate functional group.

143. The method of claim 137, wherein said (N+1)th compound comprises a free amino group.

144. The method of claim 137, wherein at least one of said tail units is linked to said chemical linker via a self-immolative spacer, the method further comprising, prior to (d):
(h) providing at least one (N+1)th compound having said self-immolative spacer linked thereto.

145. The method of claim 137, wherein said (N+1)th reactive group is selected from the group consisting of a hydroxyl, a thiol and an amine.

146. The method of claim 137, wherein said Nth compound is linked to said (N−1)th generation self-immolative dendrimer via a self-immolative spacer, the method further comprising, prior to (c):
coupling said Nth self-immolative chemical linker with said self-immolative spacer, to thereby provide said Nth compound having said Nth self-immolative chemical linker being linked to said self-immolative spacer and to at least two (N+2)th reactive groups.

147. A method of performing a diagnosis, the method comprising administering to the subject a diagnostically effective amount of the self-immolative dendrimer of claim 30.

148. The method of claim 147, wherein said at least one diagnostic agent is selected from the group consisting of a signal generator agent, a signal absorber agents and a combination thereof.

149. The method of claim 147, wherein said self-immolative dendrimer further comprises at least one self-immolative spacer.

150. The method of claim 149, wherein said spacer linking said trigger unit and said at least one self-immolative chemical linker.

151. The method of claim 149, wherein said at least one spacer linking at least one of said functional moieties and at least one of said at least one chemical linker.

152. The method of claim 149, wherein said trigger unit, said at least one spacer and said at least one self-immolative chemical linker being such that upon cleavage of said trigger unit, said at least one self-immolative chemical linker and said at least one spacer self-immolate to thereby release said functional moieties.

153. The method of claim 147, wherein said self-immolative chemical linker has a general formula selected from the group consisting of Formula Ia and Formula Ib: 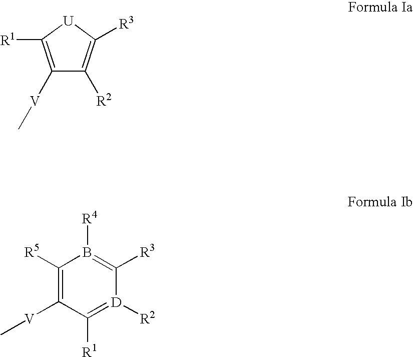
wherein:
V is O, S, PR⁶ or NR⁷;
U is O, S or NR⁸;
B and D are each independently a carbon atom or a nitrogen atom;
R¹, R², R³, R⁴ and R⁵ are each independently 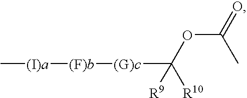
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
whereas:
a, b and c are each independently as integer of 0 to 5; and
I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R¹, R² and R³ in Formula Ia and of R¹, R², R³, R⁴ and R⁵in Formula Ib are said

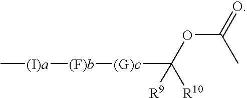

154. The method of claim 153, wherein said self-immolative chemical linker has the general Formula Ib.

155. The method of claim 154, wherein:
V is O or S;
each of B and D is a carbon atom;
each of R², R³ and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹ and R⁵ is said

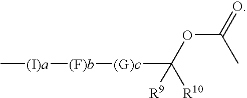

156. The method of claim 155, wherein:
each of R², R³ and R⁴ is independently hydrogen or alkyl;
each of a, b and c equal 0; and
each of R⁹ and R¹⁰ is independently hydrogen or alkyl.

157. The method of claim 154, wherein
V is O or S;
each of B and D is a carbon atom;
each of R² and R⁴ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R², R³ and R⁴ being connected to one another to form an aromatic or aliphatic cyclic structure; and
each of R¹, R³ and R⁵ is said

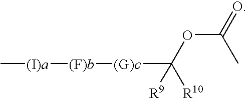

158. The method of claim 157, wherein:
each of R² and R⁴ is independently hydrogen or alkyl;
each of a, b and c equal 0; and
each of R⁹ and R¹⁰ is independently hydrogen or alkyl.

159. The method of claim 149, wherein said self-immolative spacer has a general formula selected from the group consisting of Formula IIa, Formula IIb, Formula IIc and Formula IId: 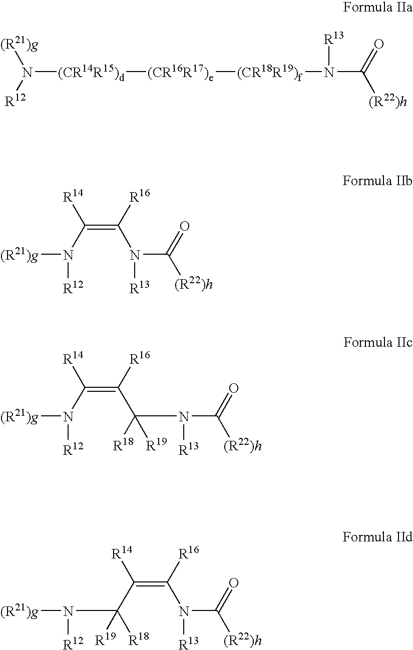
and a combination thereof,
wherein:
d, e, f, g and h and f are each independently an integer from 0 to 3, provided that d+e+f≧2;
R¹² and R¹³ are each independently hydrogen or alkyl;
R¹⁴, R¹⁵, R¹⁶, R¹⁷, R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate;
R²¹ and R²² are each independently has a general formula selected from the group consisting of Formula VIIa and Formula VIIb: 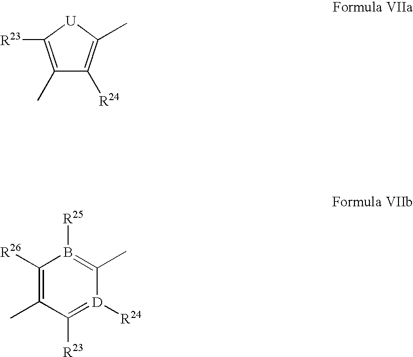
wherein:
U is O, S or NR²⁹;
B and D are each independently a carbon atom or a nitrogen atom;
R²³, R²⁴, R²⁵ and R²⁶ are each independently 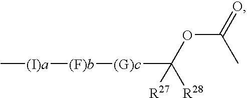
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R²³, R²⁴, R²⁵ and R²⁶ being connected to one another to form an aromatic or aliphatic cyclic structure;
whereas:
a, b and c are each independently as integer of 0 to 5; and
I, F and G are each independently —R³⁰C═CR³¹— or —C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate,
provided that at least two of R²³ and R²⁴ in Formula VIIa and of R²³, R²⁴, R³⁵ and R²⁶ in Formula VIIb are said

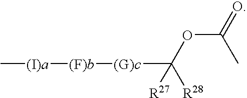

160. The method of claim 159, wherein said self-immolative spacer has the general Formula IIa.

161. A self-immolative dendrimer.
